(19) United States
(12) Patent Application Publication
(10) Pub. No.: US 2025/0117943 A1
(43) Pub. Date: Apr. 10, 2025

(54) COLONY COUNTING DEVICE AND CONTROL METHOD

(71) Applicant: Keyence Corporation, Osaka (JP)

(72) Inventors: Shingo MATSUMURA, Osaka (JP); Suguru SEKIYA, Osaka (JP)

(73) Assignee: Keyence Corporation, Osaka (JP)

(21) Appl. No.: 18/986,869

(22) Filed: Dec. 19, 2024

Related U.S. Application Data
(63) Continuation of application No. PCT/JP2023/017662, filed on May 11, 2023.

(30) Foreign Application Priority Data
Dec. 27, 2022 (JP) 2022-209788

(51) Int. Cl. G06T 7/00

(52) U.S. Cl. CPC G06T 7/0014; G06T 2200/24; G06T 2207/30024; G06T 2207/30204; G06T 2207/30242

(57) ABSTRACT

Adjustment of test parameters in a colony counting device is facilitated. A colony counting device displays a plurality of colony detection results, obtained by applying different colony detection parameters to an image of a test individual, in a comparable manner on a display section, selects one colony detection result from among the plurality of colony detection results according to an operation of a user, and applies the colony detection parameter used to obtain the selected one colony detection result to the image of the test individual to count the number of colonies included in the image of the test individual.

### CROSS-REFERENCE TO RELATED APPLICATIONS

The present application is a continuation of PCT/JP2023/017662 filed May 11, 2023, which in turn claims foreign priority based on Japanese Patent Application No. 2022-209788, filed Dec. 27, 2022, the contents of which is incorporated herein by reference.

### TECHNICAL FIELD

The invention relates to a colony counting device, a control method, and a program.

### BACKGROUND ART

In a factory that produces food, a colony counter is used to test whether or not bacteria are mixed in a product. An inspector forms a culture medium in a Petri dish, puts a food sample into the culture medium, and cultivates the food sample in a culture vessel or the like for a predetermined period. Thereafter, the inspector takes out the Petri dish from the culture vessel, and counts colonies (bacterial colonies) with the colony counter. In this manner, the counting accuracy of the colony counter is important for food hygiene management. According to Patent Literature 1, a method for counting colonies from a grayscale image is proposed.

### CITATION LIST

### Patent Literature

Patent Literature 1: JP 2012-075409 A

### SUMMARY OF INVENTION

### Technical Problem

Meanwhile, in order to accurately count colonies, a user has to set various test parameters such as a capturing condition, an illumination condition, and a detection condition of a test individual. Typically, when one parameter is changed, a colony counting device needs to capture the test individual using the new parameter to acquire a test image, and count colonies from the test image. That is, image capturing, image processing, and counting processing are repeatedly executed many times in order to adjust many parameters, and thus, the time taken for adjustment of the parameters is enormous. Therefore, an object of the present invention is to facilitate adjustment of test parameters in a colony counting device.

### Solution to Problem

The present invention provides, for example, a colony counting device including:
an acquisition section that acquires an image of a test individual obtained by capturing the test individual;
a display processing section that displays a plurality of colony detection results, obtained by applying different colony detection parameters to the image of the test individual acquired by the acquisition section, on a display section in a comparable manner;
a selection section that selects one colony detection result from among the plurality of colony detection results displayed on the display section according to an operation of a user; and
a counting section that applies the colony detection parameter used to obtain the one colony detection result selected by the selection section to the image of the test individual and counts the number of colonies included in the image of the test individual.

### Advantageous Effects of Invention

According to the present invention, the adjustment of the test parameters in the colony counting device is facilitated.

### BRIEF DESCRIPTION OF DRAWINGS

FIG. 1 is a view illustrating a colony counting device.

FIG. 2 is a view for describing a structure of a head device.

FIG. 3 is a diagram for describing an electrical configuration of the head device.

FIG. 4 is a diagram for describing an electrical configuration of a control device.

FIG. 5 is a view for describing a user interface (UI).

FIG. 6 is a view for describing a UI for creating a count table from a sample database.

FIG. 7 is a view for describing a UI related to input assistance of a sample name.

FIG. 8 is a view for describing a UI for newly creating a count table.

FIG. 9 is a view for describing a sheet of spreadsheet software.

FIG. 10 is a view for describing a UI for diverting a past count table.

FIG. 11 is a view for describing a UI for facilitating settings of statistical processing.

FIG. 12 is a view for describing a UI for facilitating the settings of statistical processing.

FIG. 13 is a view for describing a UI related to a parent-child relationship.

FIG. 14 is a view for describing a UI related to the parent-child relationship.

FIG. 15 is a view for describing a UI for adding a column element.

FIG. 16 is a view for describing a UI during a test.

FIG. 17 is a view for describing a UI changing a test condition and the like.

FIG. 18 is a view for describing a UI at the time of instructing counting.

FIG. 19 is a view illustrating a UI at the time of registering a count result in a cell.

FIG. 20 is a view for describing a UI showing automatic identification of a target cell.

FIG. 21 is a view for describing a UI for switching a unit of a count result.

FIG. 22 is a view for describing a UI for switching information to be displayed in a cell.

FIG. 23 is a view for describing a UI for re-setting a counting condition.

FIG. 24 is a view for describing a transition of assignment of functions to buttons.

FIG. 25 is a view for describing a UI for changing the test condition and the like.

FIG. 26 is a view for describing a UI for calling a count table created in the past.

FIG. 27 is a view for describing a test list.

FIG. 28 is a view illustrating a user authentication tag.

FIG. 29 is a view for describing a UI for registering information in a cell of a free column.

FIG. 30 is a view for describing a UI for registering information in a cell of a free column.

FIG. 31 is a view for describing a UI for reading of a code (symbol).

FIG. 32 is a view for describing a symbol decoding result.

FIG. 33 is a view for describing a report.

FIG. 34 is a flowchart illustrating processing executed by a PC.

FIG. 35 is a flowchart illustrating processing executed by the head device.

FIG. 36 is a flowchart illustrating editing of the sample database.

FIG. 37 is a flowchart for describing editing of the count table.

FIG. 38 is a flowchart for describing identification of the count table.

FIG. 39 is a flowchart illustrating a colony counting method.

FIG. 40 is a flowchart illustrating a method of registering information in a cell of a free column. and

FIG. 41 is a view for describing an identification image given to a Petri dish.

FIG. 42 is a schematic cross-sectional view of the head device.

FIG. 43 is a plan view for describing a coaxial illumination device including a light distribution angle regulating plate.

FIG. 44 is a cross-sectional perspective view for describing a structure of the coaxial illumination device.

FIG. 45 is a view for describing a light guide.

FIG. 46 illustrates a light distribution angle of each light emitting element.

FIG. 47 is a view illustrating that a desired light distribution angle is achieved by a lens.

FIG. 48 is a schematic cross-sectional view of a head device including a coaxial illumination device adopting another structure.

FIG. 49 is a view for describing a low degree of diffusion.

FIG. 50 is a view for describing a structure of a diffusion plate.

FIG. 51 is a view for describing a high degree of diffusion achieved mechanically.

FIG. 52 is a view for describing a low degree of diffusion achieved mechanically.

FIG. 53 is a view for describing an effect of a degree of diffusion on a test image.

FIG. 54 is a view for describing a UI for setting the degree of diffusion.

FIG. 55 is a view for describing a combined test image.

FIG. 56 is a view for describing a method of measuring an inhibition halo.

FIG. 57 is a view for describing a user interface.

FIG. 58 is a view for describing a user interface.

FIG. 59 is a view for describing a user interface.

FIG. 60 is a view for describing a user interface.

FIG. 61 is a view for describing a user interface.

FIG. 62 is a view for describing a user interface.

FIG. 63 is a view for describing a user interface.

FIG. 64 is a flowchart illustrating a parameter adjustment method.

FIG. 65 is a flowchart illustrating a parameter adjustment method.

FIG. 66 is a view for describing a user interface.

FIG. 67 is a view for describing a user interface.

FIG. 68 is a view for describing a user interface.

### DESCRIPTION OF EMBODIMENTS

Hereinafter, an embodiment will be described in detail with reference to the accompanying drawings. Note that the following embodiment does not limit the invention according to the claims, and all combinations of characteristics described in the embodiment are not necessarily essential for the invention. Two or more characteristics of the plurality of characteristics described in the embodiment may be arbitrarily combined. Further, the same or similar configurations are denoted by the same reference numerals, and redundant description will be omitted.

### [Colony Counting Device]

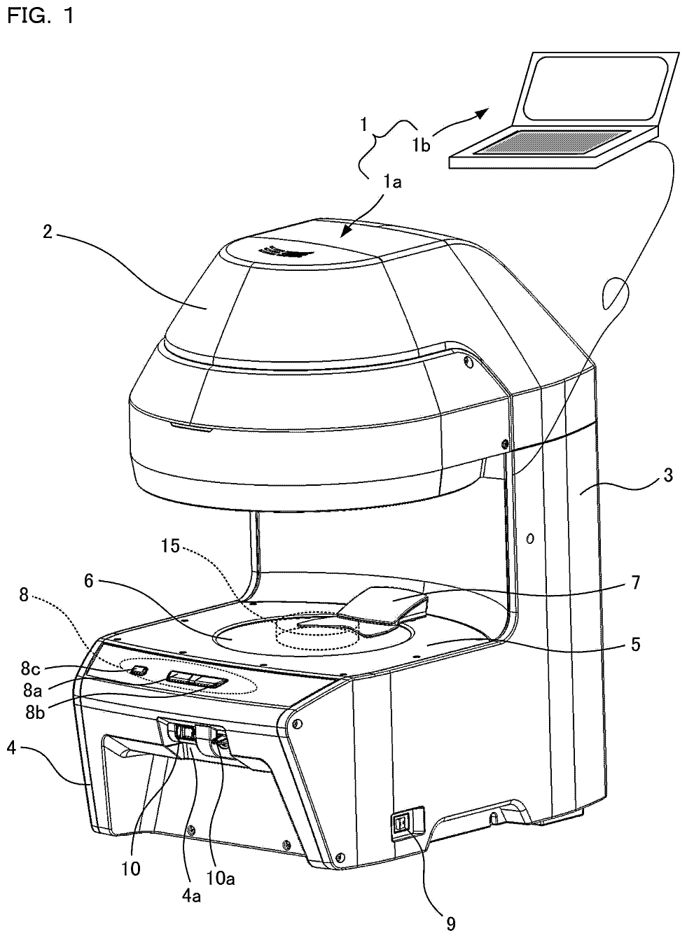

FIG. 1 illustrates a colony counting device 1. Note that the colony counting device 1 includes a head device 1a and a control device (personal computer (PC)) 1b to be described later. For example, the head device 1a and the PC 1b may be connected to each other in a wired manner by a universal serial bus (USB) cable, or may be connected to each other in a wireless manner.

The head device 1a includes an upper unit 2, a support unit 3, and a lower unit 4. A camera and an illumination device are provided inside the head device 1a. The support unit 3 exists between the upper unit 2 and the lower unit 4, and supports the upper unit 2. A stage 5 is provided on a top surface of the lower unit 4. The stage 5 is provided with a transmission window 6 on which a Petri dish 15 is placed and a positioning member 7 configured to position the Petri dish 15 at the center of the transmission window 6. An operation section 8 and a front camera 10 are provided in front of the lower unit 4. The operation section 8 includes a plurality of switches (for example, a first hardware button 8a, a second hardware button 8b, and a third hardware button 8c) configured for a user to input instructions. The front camera 10 is optional, and reads, for example, a two-dimensional symbol (barcode) and the like. The front camera 10 is arranged in a recess 4a provided in a front surface of a housing of the head device 1. A switching lever 10a is a lever for switching the orientation of the front camera 10 forward or downward. A power switch 9 is provided on a side surface of the lower unit 4.

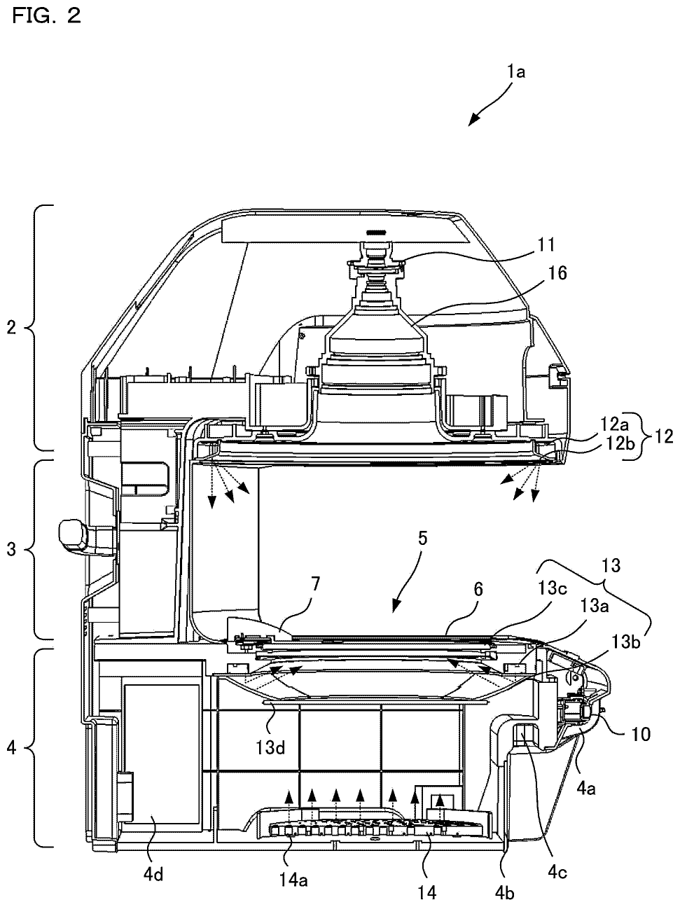

FIG. 2 is a cross-sectional view of the head device 1a. A ring illumination device 12 configured to perform epi-illumination is arranged near a lower surface of the upper unit 2. The epi-illumination is an illumination technique for observing a test individual by receiving light reflected by the test individual. A main camera 11 and an optical system 16 are arranged above the ring illumination device 12. A ring illumination device 13 is also arranged below the transmission window 6. A coaxial illumination device 14 that performs coaxial illumination (total illumination) is arranged below the ring illumination device 13. The coaxial illumination (total illumination) is also called transmitted illumination, and is used in a technique for observing a test individual by receiving light transmitted through the test individual. The ring illumination device 12 includes a plurality of light emitting elements 12a arranged in a ring shape, and a diffusion plate 12b that diffuses light output from the plurality of light emitting elements 12a. An illumination direction can be freely changed by selecting the light emitting elements 12a to be simultaneously turned on. This may be useful in combining a plurality of test images acquired by capturing images of a test individual (food sample) illuminated from different directions. The ring illumination device 13 includes a plurality of light emitting elements 13a arranged in a ring shape, a reflection plate 13b, and a diffusion plate 13c. The reflection plate 13b reflects light output from the plurality of light emitting elements 13a toward the diffusion plate 13c.

The diffusion plate 13c uniformly diffuses the light from the reflection plate 13b. The diffusion plate 13c may be a diffusion member capable of electrically adjusting a degree of diffusion. A second diffusion plate 13d is provided below the diffusion plate 13c. The diffusion plate 13d is a diffusion member having a constant degree of diffusion.

An illumination direction is freely changed by selecting the light emitting elements 13a to be simultaneously turned on among the plurality of light emitting elements 13a. The coaxial illumination device 14 includes a plurality of light emitting elements 14a arranged concentrically or in an array. As a plurality of types of the illumination devices are provided in this manner and an appropriate illumination device is selected for each combination of a food sample and a culture medium, the number of colonies may be accurately countable.

The lower unit 4 has a front pillar 4b and a rear pillar 4d which are made of metal and instruct the stage 5. A part of the front pillar 4b has a cross-sectional shape like an inverted Greek letter f. That is, the front pillar 4b has a recess 4c functioning as a grip portion into which the user can insert a finger. Since the recess 4c is provided in the front pillar 4b, the head device 1a is hardly distorted even if the user puts his/her hand into the recess 4c and lifts the head device 1a. Therefore, the user can stably carry the head device 1a.

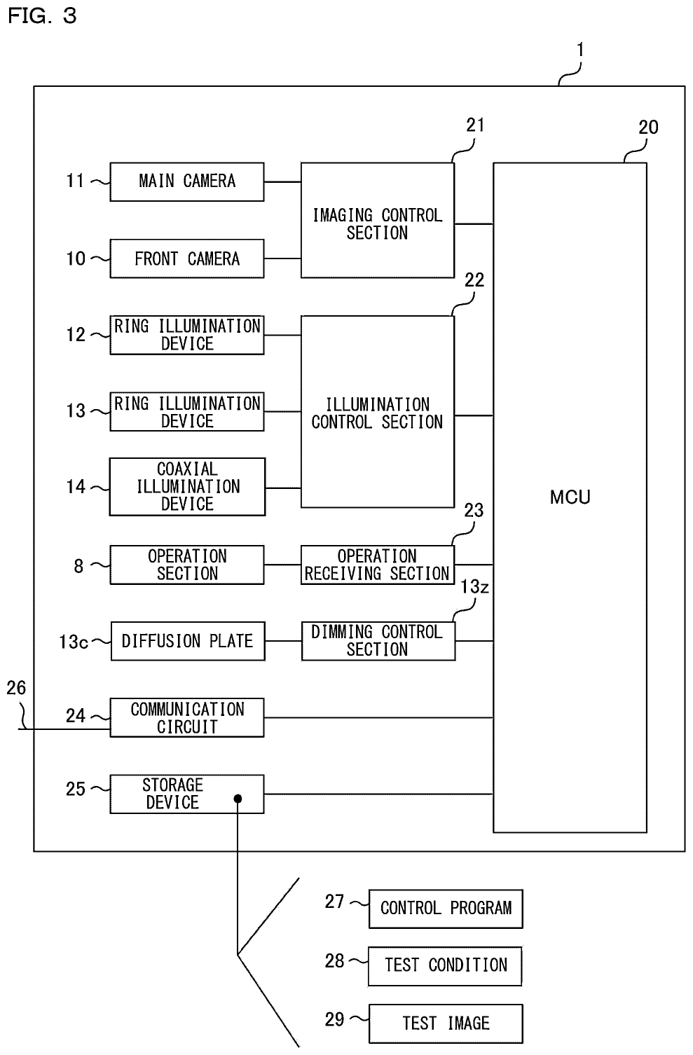

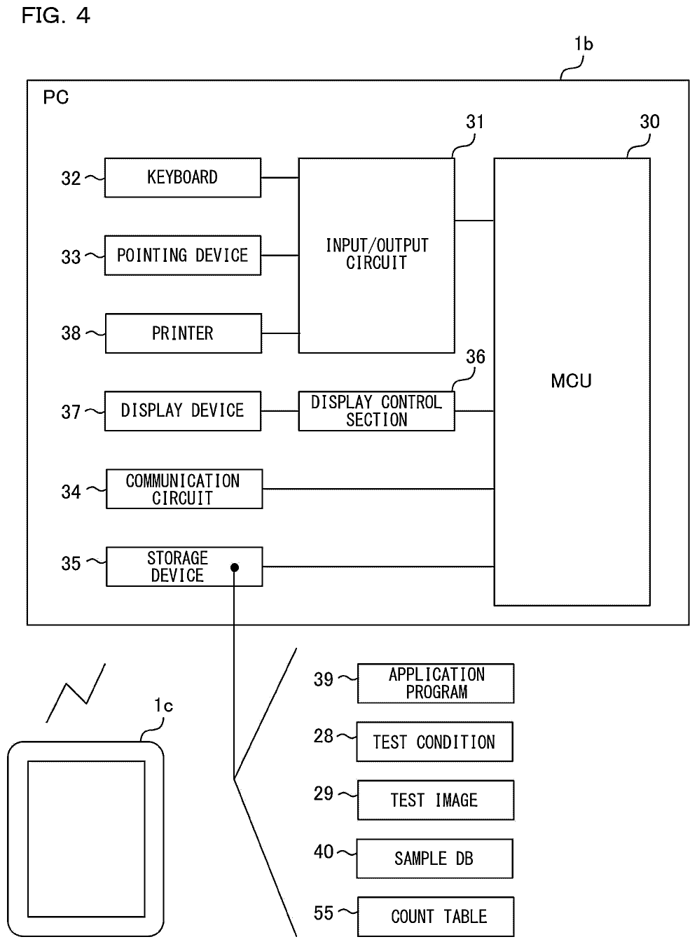

FIG. 3 illustrates an electrical configuration of the head device 1a. An MCU 20 is a processor that executes a control program 27 stored in a storage device 25 and controls the head device 1a according to the control program 27. Note that MCU is an abbreviation for micro-controller unit. The MCU 20 controls the main camera 11 and the front camera 10 via an imaging control section 21 to acquire various types of image data. The imaging control section 21 controls, for example, an exposure time of the main camera 11. The MCU 20 turns on or off the ring illumination devices 12 and 13 and the coaxial illumination device 14 via an illumination control section 22. The illumination control section 22 controls driving power to be supplied to the ring illumination devices 12 and 13 and the coaxial illumination device 14. The MCU 20 receives a user input that is input from the operation section 8 via an operation receiving section 23. The operation receiving section 23 includes an input circuit or the like that generates a signal indicating a state of a switch section of the operation section 8. A communication circuit 24 is a circuit that communicates with the PC 1b illustrated in FIG. 4 via a communication cable 26. The communication circuit 24 may include a wireless communication circuit and a LAN interface circuit. LAN is an abbreviation for local area network. The communication cable 26 may be, for example, a USB cable. The storage device 25 includes, for example, a read-only memory (ROM) that stores the control program 27 and a random access memory (RAM) used as a work area. The storage device 25 may store, for example, a test condition 28 set by the PC 1b, a test image 29 acquired by the main camera 11, and the like. The test condition 28 can include, for example, an illumination condition, an imaging condition, a count condition, and the like. The test image 29 is an image of the Petri dish 15 including a culture medium and a sample.

The dimming control section 13z electrically controls the degree of diffusion of the diffusion plate 13c in accordance with a command from the MCU 20.

FIG. 4 illustrates the PC 1b that controls the head device 1a. An MCU 30 is a processor that executes a program stored in a storage device 35 and controls the PC 1b and the head device 1a according to the program. The MCU 30 receives a user instruction from a keyboard 32 and a pointing device 33 connected to an input/output circuit 31. The MCU 30 controls a printer 38 connected to the input/output circuit 31 to print a table or the like on paper. The MCU 30 displays various types of information on a display device 37 via a display control section 36 such as a graphics board. A communication circuit 34 is a circuit that communicates with the head device 1a via the communication cable 26. The communication circuit 34 may include a wireless communication circuit and a LAN interface circuit. The storage device 35 includes, for example, a read-only memory (ROM) that stores a program and a random access memory (RAM) used as a work area. Moreover, the storage device 35 may include a hard disk drive (HDD) and a solid state drive (SSD). The storage device 35 may store an application program 39, the test condition 28, the test image 29, a sample DB 40, a count table 55, and the like. DB is an abbreviation for database. The application program 39 is in charge of, for example, creation and editing of the sample DB 40 and the count table 55, control of the head device 1, and the like. The test condition 28 is set by the MCU 30 according to the application program 39. The test image 29 is received from the head device 1. The sample DB 40 is a database referred to when the count table 55 is created. The count table 55 is a table in which a plurality of cells are arranged in an array, and includes a row element and a column element.

In the PC 1b, the communication circuit 34 may execute wireless communication with a terminal device 1c such as a smartphone or a tablet terminal. The terminal device 1c may display the count table 55 or may display a test list created from the count table 55. The test list includes a Petri dish number, a sample name, a bacterial species, a culture medium, a dilution factor, a culture time, and the like, and is referred to when the user prepares a test individual in the Petri dish 15.

### [Test Procedure]

A general test procedure is as follows.
(1) The user creates a test list by handwriting. The test list includes a plurality of rows, and a Petri dish number, a bacterial species, a dilution factor, a count number, and a comment (a sample product name and the like) can be written in each of the rows. Note that the Petri dish number referred to here is identification information assigned in advance according to a predetermined rule in order to specify a culture condition such as a type of a culture medium or a dilution factor.
(2) The user writes numbers onto lids of Petri dishes according to the test list, or writes numbers written in advance on the Petri dishes into the test list.
(3) The user creates the culture medium according to the dilution factor written in the test list. If the dilution factor is not written in the test list, the user writes an actual dilution factor in the test list.
(4) The user puts (mixes) a sample into the culture medium of each of the Petri dishes. The user writes a name of the sample in a field of the comment of the test list.
(5) The user puts the Petri dishes into a culture vessel.
(6) When a predetermined time has elapsed, the user takes out the Petri dishes from the culture vessel and counts the number of colonies. For example, the user gives a counted mark with an oil-based pen to a position of a colony while looking through the colony from a bottom surface side of the Petri dish. The number of colonies is written in the test list. Note that the user may count the number of colonies for each bacterial species while visually confirming the bacterial species. In this case, the user writes the number of colonies for each bacterial species into the test list for each of the Petri dishes.
(7) The user activates the PC, and reads and inputs numerical values and characters written in the test list to spreadsheet software. The number of colonies is aggregated using a macro function of the spreadsheet software or the like.

In this manner, the test list is created by handwriting in the conventional test procedure, which is extremely troublesome work for the user. Further, if there is an erroneous input when the numerical values or the like written in the test list are transcribed to a sheet of the spreadsheet software, there is a possibility that an aggregation result is also erroneous. Even if the number of colonies can be automatically acquired by a colony counter, there is still a possibility of erroneous writing and erroneous input since all of the creation of the test list, the writing of the number of colonies into the test list, and the transcription from the test list to the sheet of the spreadsheet software are handwritten in the conventional technique.

Therefore, in the present example, it is proposed that an electronic test list is created by the PC 1b, colonies are counted according to an electronic test list, a counting result is directly input to the electronic test list, and input numbers are aggregated. As a result, burden on the user regarding post-processing on colony counting results may be mitigated. Further, the erroneous input may also be reduced, and test accuracy may be improved since handwriting or manual input by the user is reduced.

### [Creation of Test List (Count Table)]

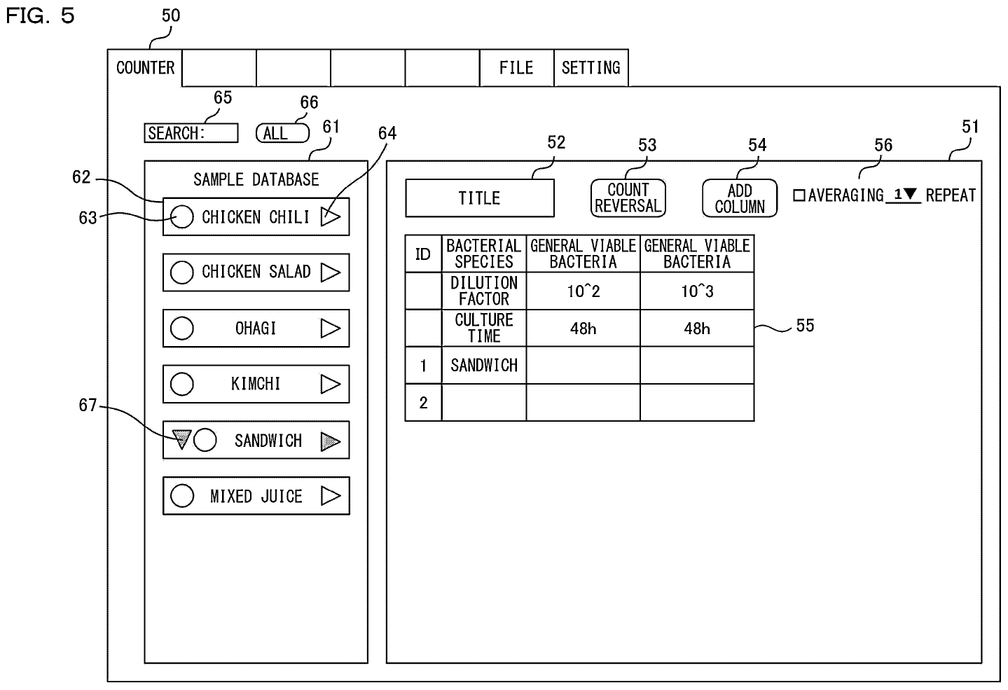

FIG. 5 illustrates an UI 50 of a count application program displayed on the display device 37 of the PC 1b. The count application program is stored in the storage device 35 and executed by the MCU 30. The UI 50 includes a button, a link, a tab, and the like for switching a plurality of functions included in the count application program.

The UI 50 includes a table creation area 51 and a DB display area 61. DB is an abbreviation for database. The table creation area 51 displays at least the count table 55. A title display section 52 receives and displays an input of a title (name) given to the count table 55 from the keyboard 32. A button 53 is a button for switching execution/non-execution of a count of each cell. A button 54 is a button for instructing addition of a column to the count table 55. The averaging setting section 56 includes a check box for instructing whether or not to execute averaging of count results, and a selection section of the number of count values to be averaged (=the number of iterations of the count).

The DB display area 61 displays a list of templates (for example, the sample DB 40) of count items registered in advance. Here, the count item corresponds to one row in the count table 55. The count item is typically distinguished by a name (sample name) of a test target object. A name display section 62 displays a name (sample name) of a template registered in advance. An indicator 63 is an object that visually displays a classification tag associated with the sample name. The classification tag is a tag indicating a classification (for example, a staple, a side dish, or a dessert) defined by a user. For example, the indicator 63 may represent a difference in the classification tag using a difference in a color. The indicator 63 may represent a difference in the classification tag using a difference in a shape of the indicator 63. A button 67 is a button for expanding and displaying one or more sub-items having a parent-child relationship with respect to a certain sample name. The parent-child relationship refers to a relationship between a sample and a plurality of ingredients constituting the sample. For example, when a sandwich is used as a parent, ingredients (for example, ham, lettuce, and egg) constituting the sandwich are children. A button 64 is a button for instructing addition of a corresponding template to the count table 55. Since the sample DB 40 is prepared in advance in this manner, the user can easily create the count table 55.

In a case illustrated in FIG. 5, when the user presses the button 64 associated with a sandwich, the MCU 30 adds a row to the count table 55, displays “1” as an ID in an ID display cell of the added row, and displays “Sandwich” in a cell displaying a sample name in the added row. The ID is an abbreviation for identification information. That is, the ID and the sample name are set on a row-basis, and are “settings for the row”. Moreover, the MCU 30 reads a test condition of the sandwich registered in the sample DB 40 from the storage device 35, adds a new column to the count table 55, and displays the read test condition in the new column. In this example, the test condition includes a bacterial species (for example, general viable bacteria or Escherichia coli), a dilution factor of a culture medium, a culture time of a sample, and the like. Each column includes, for example, a cell for a bacterial species, a cell for a dilution factor, a cell for a culture time, and a cell for a count value. That is, the bacterial species, the dilution factor, and the culture time are set on a column-basis, and are “settings for the column”. In this example, nothing is input to the cell for the count value since a test has not been executed yet and the count value has not been obtained. The first test item for the sandwich is that a culture medium having a dilution factor of 100 times is used for general viable bacteria and a culture time of 48 hours is applied. The second test item for the sandwich is that a culture medium having a dilution factor of 1000 times is used for general viable bacteria and a culture time of 48 hours is applied. In this manner, the MCU 30 adds columns in accordance with the number of the test items.

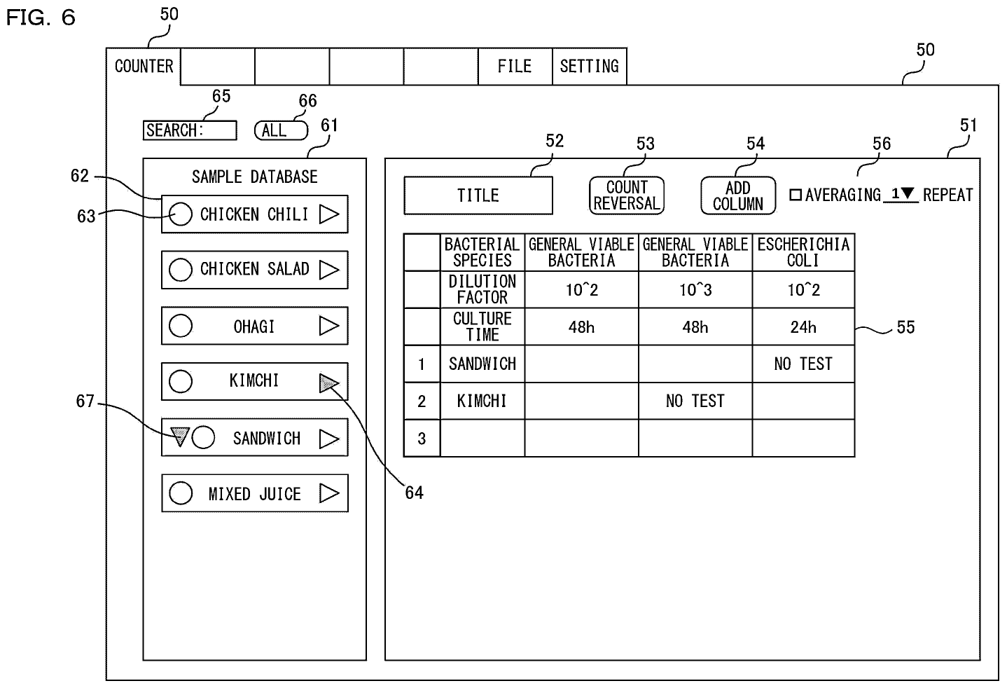

In FIG. 6, when the user presses the button 64 associated with Kimchi, the MCU 30 reads a test condition of the Kimchi registered in the sample DB 40 from the storage device 35, and adds a row and a column corresponding to the read test condition to the count table 55.

In this example, the Kimchi has two test items. The first test item for the Kimchi is that a culture medium having a dilution factor of 100 times is used for general viable bacteria and a culture time of 48 hours is applied. This is common to the first test item for the sandwich. Therefore, the MCU 30 discards the first test item included in a template of the Kimchi and does not add the test item as a new column. The second test item for the Kimchi is that a culture medium having a dilution factor of 100 times is used for Escherichia coli, and a culture time of 24 hours is applied. The MCU 30 adds this as a new column to the count table 55.

Note that a test for Escherichia coli is not performed for the sandwich. Therefore, characters or an image indicating “No test” may be displayed in the cell for the count value. Similarly, a test using the culture medium having the dilution factor of 1000 for general viable bacteria is not performed for the Kimchi. Therefore, the characters or the image indicating “No test” may be displayed in the cell for the count value.

Note that the execution/non-execution of a count can also be executed by operating a count reversal button 53. For the sandwich, when the count reversal button 53 is operated in a state in which the cell corresponding to Escherichia coli is selected, the MCU 30 may be capable of switching between displaying the characters or the image indicating “No test” and leaving a blank to input a count result.

As illustrated in FIGS. 5 and 6, a search box 65 and a tag search narrowing button 66 may be added. When the number of templates registered in the sample DB 40 increases, it becomes difficult for the DB display area 61 to display all the templates at a time. Therefore, the MCU 30 may search the storage device 35 based on characters input to the search box 65 to extract a template, and display a search result in the DB display area 61. Further, when the tag search narrowing button 66 is pressed, the MCU 30 may display only a sample product filtered by a designated classification tag. For example, the same classification tag may be given to a plurality of sample products. In this case, a plurality of sample products given with the selected classification tag are added to the count table 55.

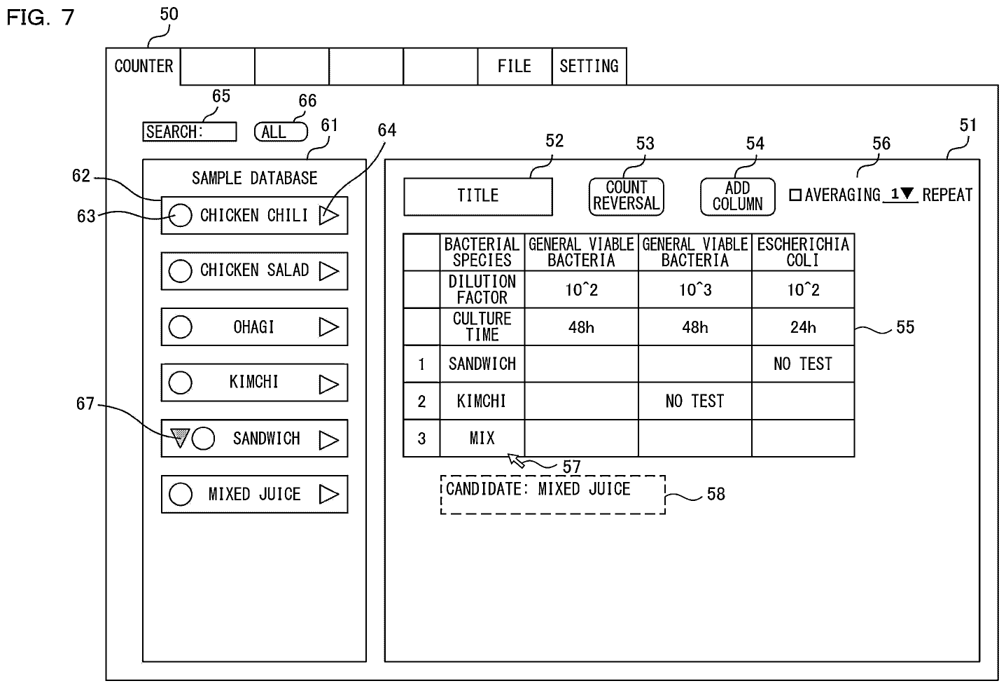

FIG. 7 illustrates another procedure of adding a new row. The user selects a sample name display cell in the new row with a pointer 57, and sequentially inputs a sample name from the first character through the keyboard 32. The MCU 30 searches the sample DB 40 with one or more input characters, and displays sample names as input candidates on a candidate display section 58. In this example, since “Mix” is input in the sample name display cell, the MCU 30 searches for a template including “Mix” as a sample name, finds “Mixed juice”, and displays “Mixed juice” on the candidate display section 58. Here, when the user selects “Mixed juice” displayed on the candidate display section 58 by the pointer 57, the MCU 30 reads a test condition of a mixed juice from the storage device 35 and adds a column according to the read test condition. As a result, the search condition is associated with a cell of the mixed juice.

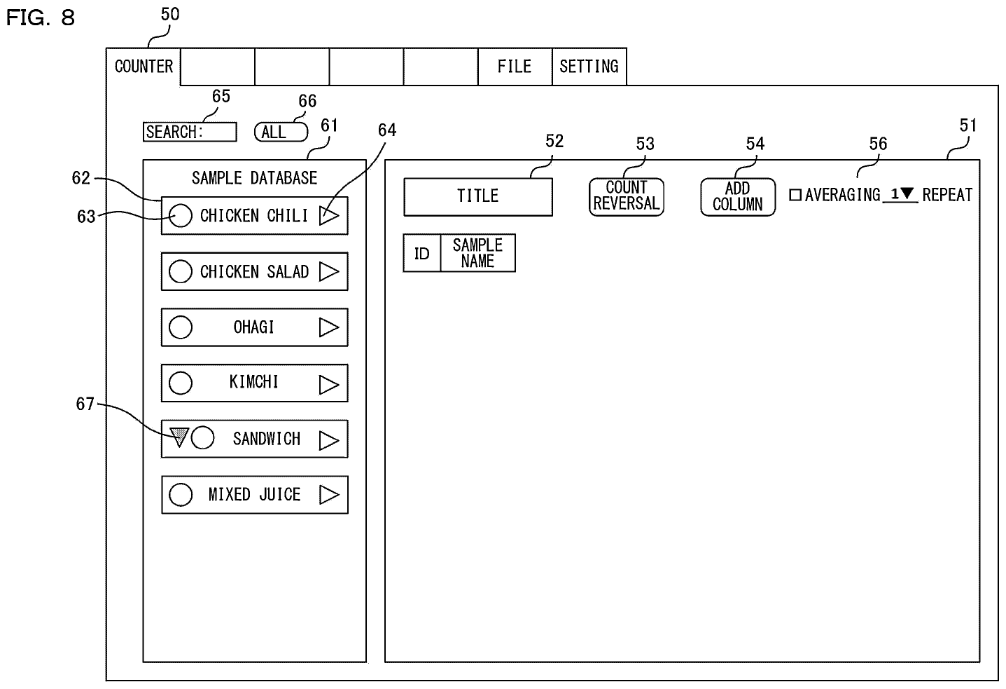

FIG. 8 illustrates the UI 50 in a case where a count table is newly created. For example, the MCU 30 can start spreadsheet software in parallel with the application program 39.

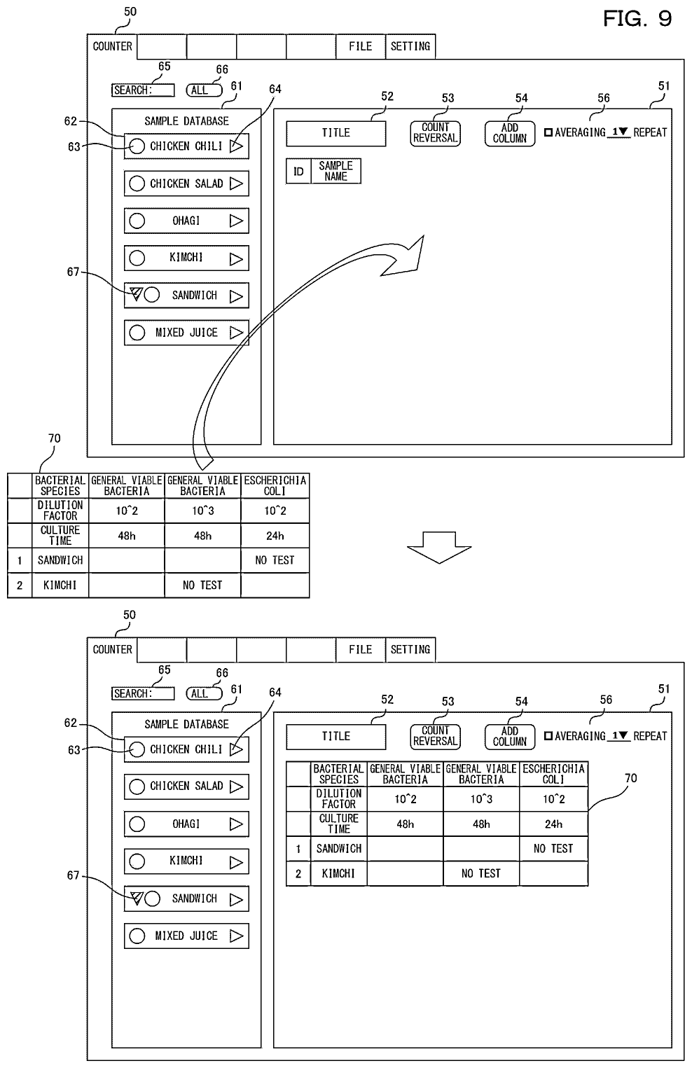

FIG. 9 illustrates a sheet 70 of the spreadsheet software. The MCU 30 receives a copy and paste instruction to the UI 50 for the sheet 70 or a cell group selected in the spreadsheet software. As a result, the MCU 30 may create the count table 55 illustrated in FIG. 6.

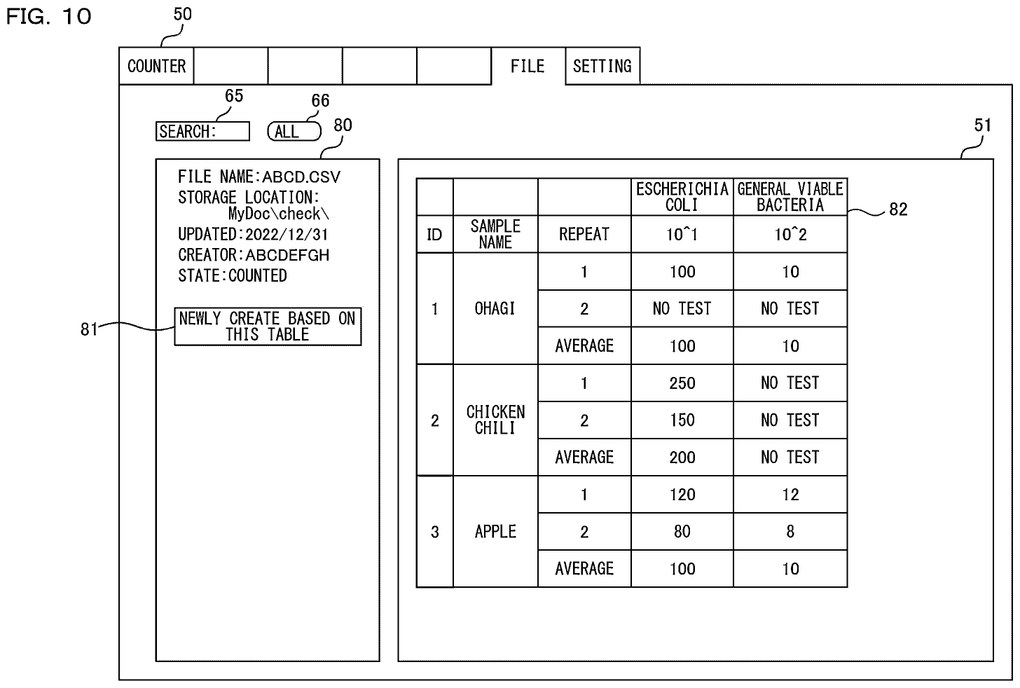

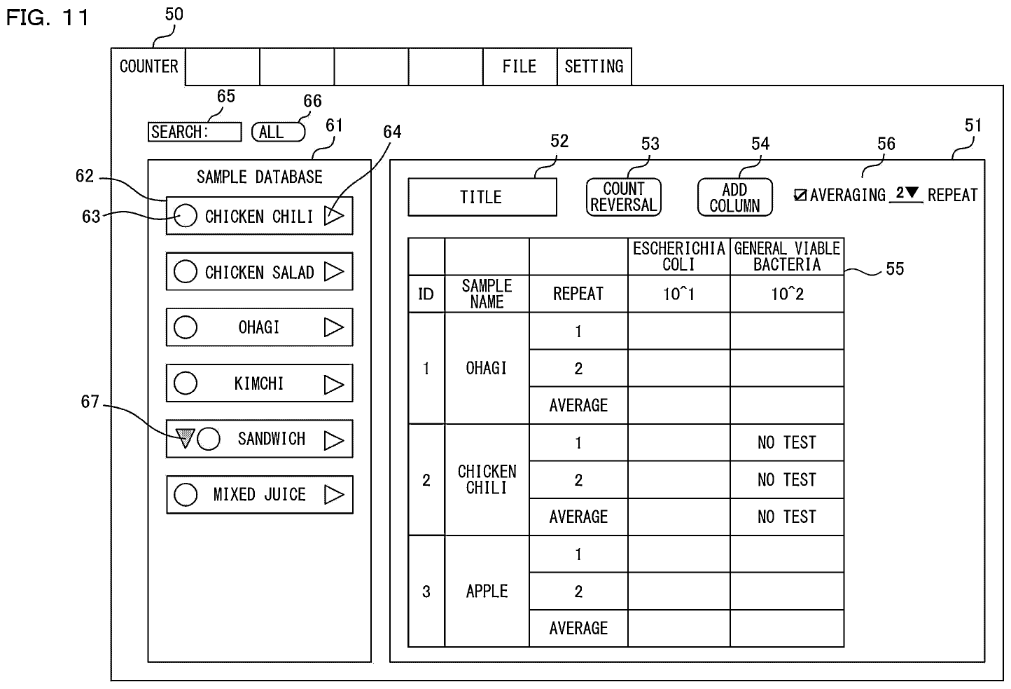

FIG. 10 is a view for describing a procedure of calling a count table 82 to which count values have already been input and creating a new count table 55. The MCU 30 may read a file corresponding to a file name input to the search box 65 from the storage device 35 and display an attribute of the file on an attribute display section 80. The MCU 30 displays the read count table 82 in the table creation area 51. As illustrated in FIG. 10, the count values have already been input to the cells for the count values. When sensing that a new creation button 81 is pressed, the MCU 30 deletes all the count values input to the cells for the count values constituting the read count table 82 and creates the new count table 55. FIG. 11 illustrates the new count table 55 created from the count table 82 to which the count values have already been input. Instead of deleting the count values, the count values may be reset to zero.

Note that the MCU 30 may create one count table 55 by merging a plurality of count tables 82. In this case, the MCU 30 analyzes the plurality of count tables 82, deletes overlapping rows and columns, and creates the new count table 55.

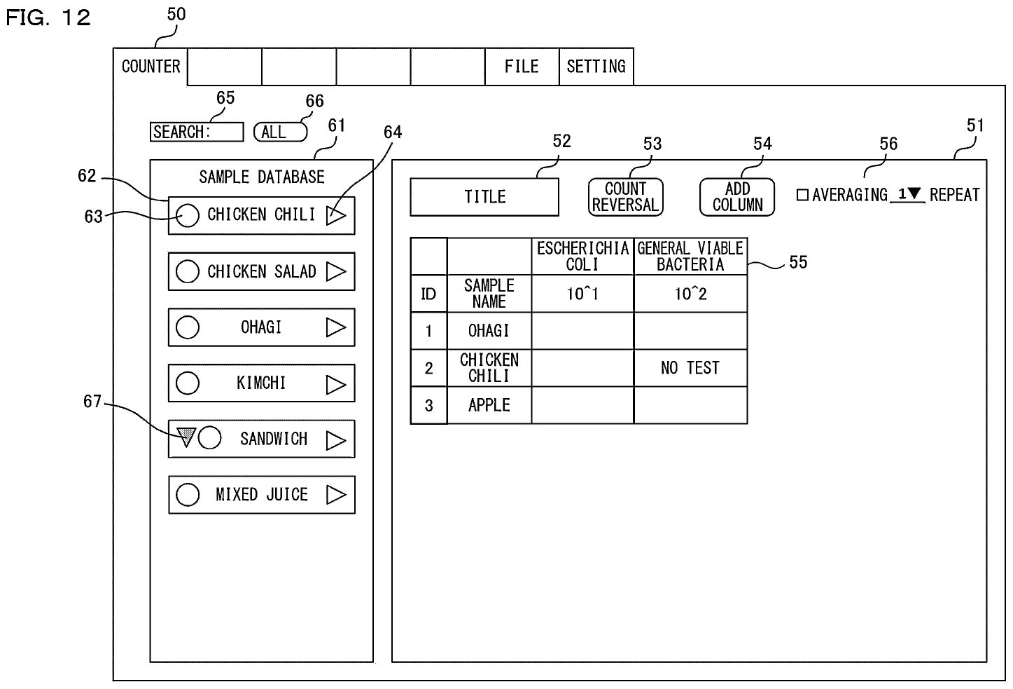

FIG. 12 is a view for describing a method of creating the count table 55 including averaging. In this example, since averaging has not yet been applied, one row is provided for one sample name. In this state, when the averaging setting section 56 is checked and “2” is selected as the number of iterations, the MCU 30 displays the UI 50 illustrated in FIG. 11. As illustrated in FIG. 11, since the number of iterations is “2”, the MCU 30 divides an input row for a count value associated with each sample name into three rows, and adds a column indicating the number of iterations. In this example, among the three rows, the first row has a cell to which a count value acquired in the first test is input. The second row has a cell to which a count value acquired in the second test is input. The third row has a cell to which an average value of the count value acquired in the first test and the count value acquired in the second test is input. In this manner, the MCU 30 may add the cells to which the count values are input and the cell to which the average value is input to the count table 55 in accordance with the number of iterations.

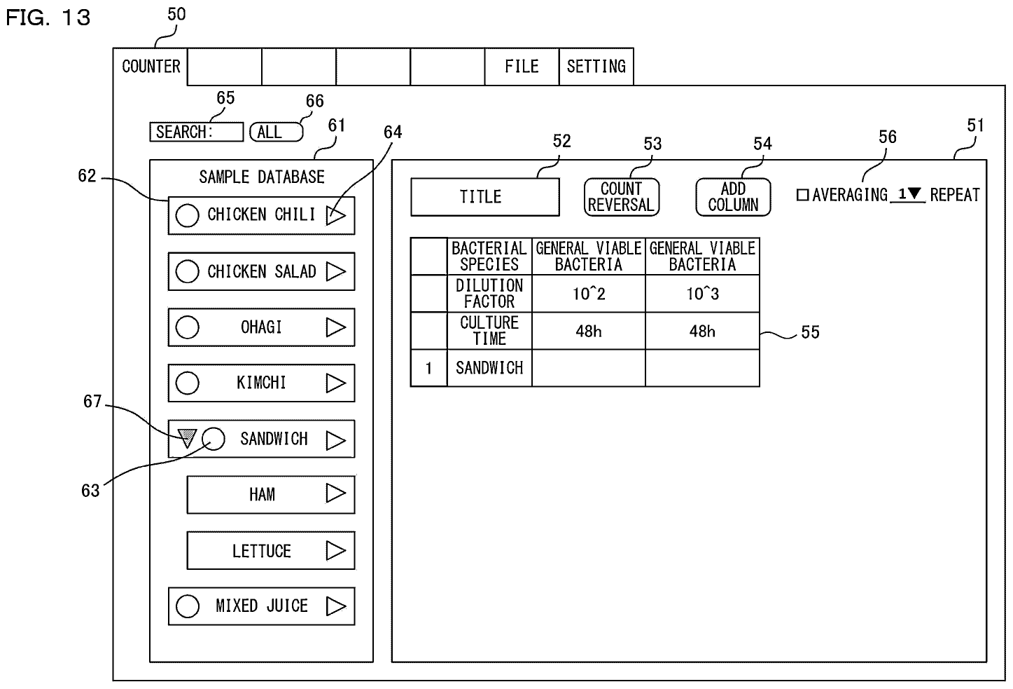

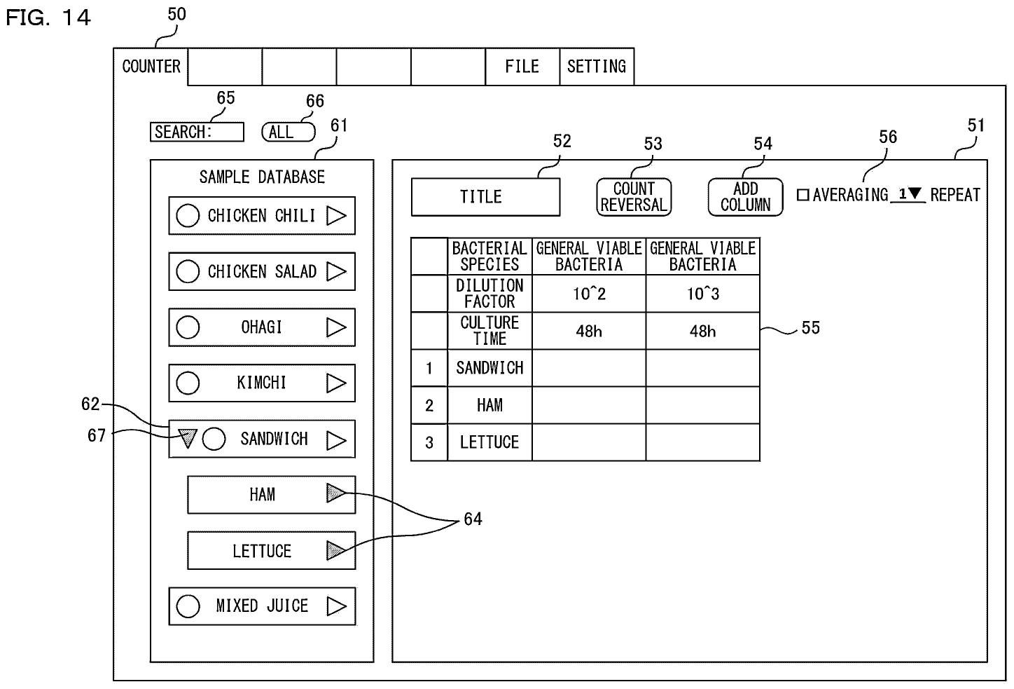

FIG. 13 illustrates an example of a parent-child relationship among a plurality of samples. In this example, when the indicator 63 of the sandwich is pressed, the MCU 30 selectively displays “ham” and “lettuce”, which are ingredients having the parent-child relationship with the sandwich, in the DB display area 61. In this state, when the button 64 of “ham” or “lettuce” is pressed, the MCU 30 reads a test item of “ham” or “lettuce” from the storage device 35 and adds the test item to the count table 55. FIG. 14 illustrates that the respective test items “ham” and “lettuce” are read from the storage device 35 and added to the count table 55. The sandwich, the ham, and the lettuce having the parent-child relationship may be collectively registered. For example, when the button 64 associated with the sandwich is pressed, the sandwich, the ham, and the lettuce may be collectively registered in the count table 55.

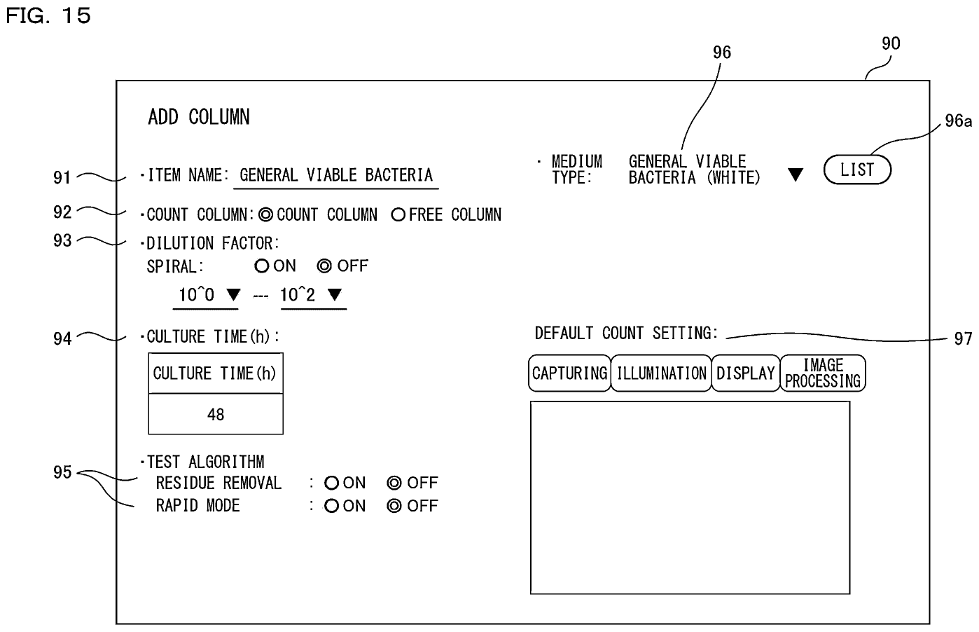

FIG. 15 illustrates a dialog 90 for adding a column. When the button 54 provided on the UI 50 is pressed, the MCU 30 displays the dialog 90 on the display device 37. The item name setting section 91 receives an input of a name of a bacterial species which is an item name of a column. A column type setting section 92 receives a setting as to whether or not a column type is a count column or a free column. The count column is a column including a cell to which a count value is input. The free column is a column in which the user can freely input text, an image, and other information such as a remark and a comment. A dilution factor setting section 93 receives an input of a dilution factor. A culture time setting section 94 receives an input of a culture time. An algorithm setting section 95 receives a setting of image processing to be applied to a test image. Residue removal is a mode in which residues (for example, dirt, stain, and handwriting) attached to the Petri dish 15 or the like are reduced by image processing. A rapid mode is a mode in which rapid result confirmation is emphasized, and is a mode in which the Petri dish 15 cultured in a culture time shorter than that in the related art is tested with higher sensitivity. A culture medium type setting section 96 receives selection of a culture medium type (for example, general viable bacteria (white) or a general viable bacteria (black)) and the like. Note that, when a list button 96a is pressed, the MCU 30 may read culture medium type candidates from the storage device 35 to create a list, and display the list on the display device 37. A count setting section 97 receives a setting of a capturing condition (for example, exposure time) of the main camera 11, a type of illumination (for example, brightness and an illumination device), a type of display processing, a type of image processing, and the like. That is, the bacterial species, the dilution factor, the culture time, the algorithm setting, the culture medium type, or the count setting that has been received in the dialog 90 are set for each column as default settings.

As will be described later, a UI for receiving selection of an illumination type, a setting of brightness, a setting of a degree of diffusion, and the like may be displayed on the display device 37 by pressing an illumination button of the count setting section 97.

Further, the storage device 35 may store a plurality of degrees of diffusion associated with candidates for the culture medium type. In this case, the MCU 30 can read and acquire a degree of diffusion associated with a selected culture medium from the storage device 35.

### [Count Processing]

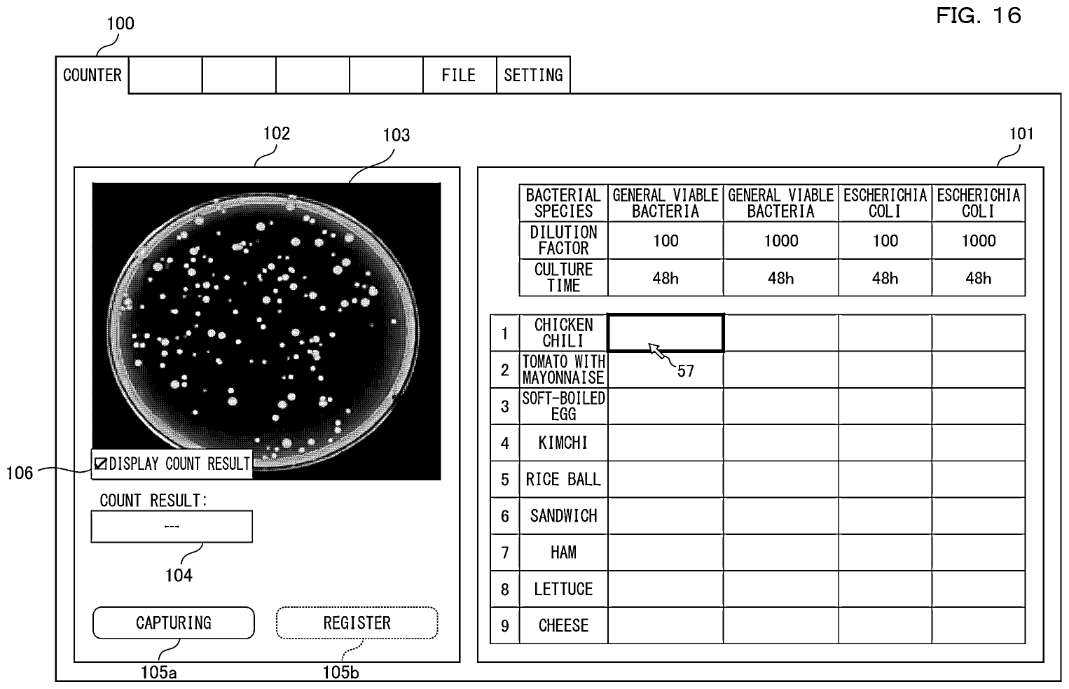

FIG. 16 illustrates a UI 100 displayed on the display device 37 of the PC 1b during execution of count processing by controlling the head device 1a from the PC 1b. A count table area 101 is an area for displaying the count table 55 edited through the UI 50. The result area 102 is an area for displaying a test image 103 acquired by the main camera 11 of the head device 1a. Note that the test image 103 may be a moving image or a still image. In general, the MCU 30 acquires a moving image by the main camera 11 and displays the moving image in the result area 102 when adjustment of the exposure time of the main camera 11, the brightness of each of the ring illumination devices 12 and 13 and the coaxial illumination device 14, selection of light emitting elements to be turned on, selection of the image processing, and the like are executed. On the other hand, the MCU 30 acquires a still image by the main camera 11 and displays the acquired still image in the result area 102 when the count processing is executed.

A check box 106 is a control object for selecting whether or not to display a count result in the count value area 104. A first software button 105a is a button having the same function as the first hardware button 8a. A second software button 105b is a button having the same function as the second hardware button 8b. In this example, a capturing instruction (capture button) is assigned to the first software button 105a. A registration instruction (register button) is assigned to the second software button 105b. In FIG. 15, the second software button 105b is indicated by a broken line, which means being inoperable.

A user clicks and selects a cell corresponding to the Petri dish 15 set on the stage 5 among a plurality of cells included in a count table displayed in the count table area 101 with the pointer 57. As illustrated in FIG. 16, the cell selected by the pointer 57 may be displayed in an emphasized manner such that any cell that has been selected by the user can be recognized. Each cell is stored in the storage device 35 in association with a test condition (sensitivity when binarizing a colony, an illumination device type, brightness, and the like) in advance. The MCU 30 reads the test condition associated with the selected cell from the storage device 35 and transmits the test condition to the head device 1a. The MCU 20 of the head device 1a controls the main camera 11, the ring illumination devices 12 and 13, and the coaxial illumination device 14 according to the received test condition, acquires an image, and transmits the image to the PC 1b. Note that, when another cell is selected, the MCU 30 reads a test condition associated with the selected cell from the storage device 35 and transmits the test condition to the head device 1a. The MCU 20 of the head device 1a controls the main camera 11, the ring illumination devices 12 and 13, and the coaxial illumination device 14 according to the received test condition, acquires an image, and transmits the image to the PC 1b. In this manner, the user can change the test condition by selecting the cell.

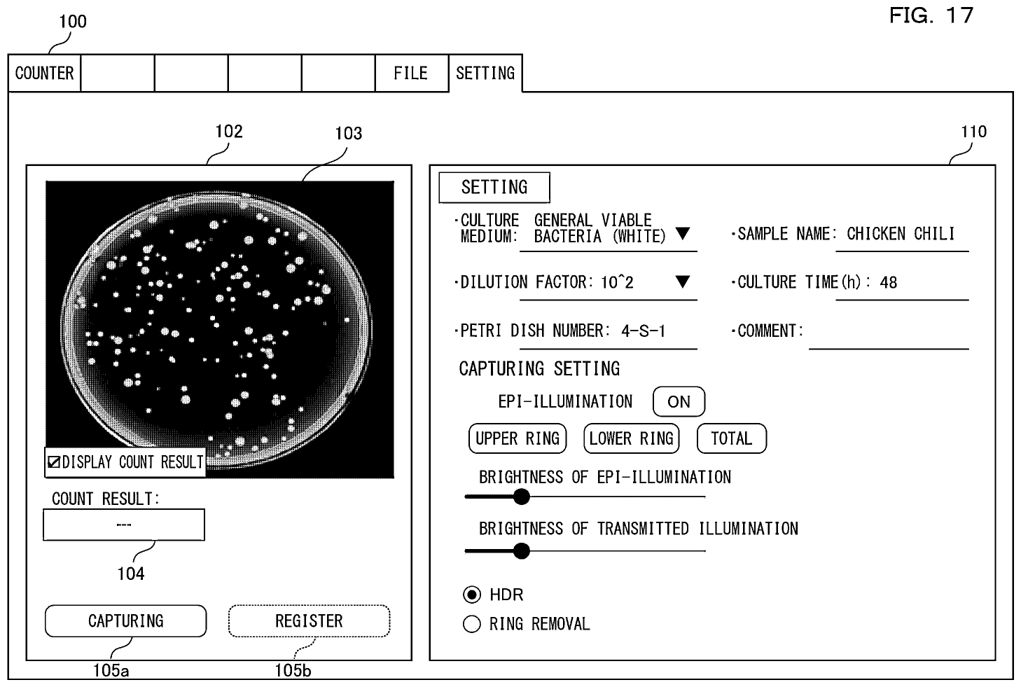

FIG. 17 illustrates a confirmation screen 110 for a test condition displayed by clicking a setting tab or a setting button on the pointer 57. As a result, the user can confirm the test condition for each cell. Note that the confirmation screen 110 may be displayed by right clicking a cell with the pointer 57. Examples of information displayed on the confirmation screen 110 include a sample name, a type of a culture medium, a dilution factor, a culture time, a Petri dish number, a comment, capturing settings (on/off of epi-illumination, an illumination device type, brightness of epi-illumination, brightness of transmitted illumination, image processing (count settings such as on/off of high dynamic range “HDR” and on/off of ring removal), and the like. In FIG. 17, “Upper ring” is an abbreviation for the ring illumination device 12. “Lower ring” is an abbreviation for the ring illumination device 13. “Total” is an abbreviation for the coaxial illumination device 14. Here, the MCU 30 sets default values on a column-basis on the dialog 90 illustrated in FIG. 15 for a bacterial species, the dilution factor, the culture time, an algorithm setting, the type of the culture medium, or the capturing settings (count settings), but may individually set a default value for each cell on the confirmation screen 110 of FIG. 17.

The confirmation screen 110 may have a pull-down list, a check box, or a radio button for selecting the degree of diffusion. Alternatively, the MCU 30 may read the degree of diffusion associated with the culture medium type selected on the confirmation screen 110 from the storage device 35 and associate the read degree of diffusion with the cell. When the degree of diffusion is changed on the confirmation screen 110, the MCU 30 sets the changed degree of diffusion in the dimming control section 13z, and the dimming control section 13z changes the diffusion of the diffusion plate 13c. The test individual is irradiated with test light diffused with the changed degree of diffusion is emitted to the test individual, and is captured by the main camera 11. The test image 103 generated by the main camera 11 is displayed in the result area 102. As a result, the user can determine which degree of diffusion is appropriate while observing the test image 103.

Note that the confirmation screen 110 displaying a list of settings corresponding to a cell may be displayed to be superimposed on the UI 100 by receiving a specific input such as a double click on the cell. Further, when a cell is selected by the pointer 57, the MCU 30 may display settings corresponding to the cell on the UI 100.

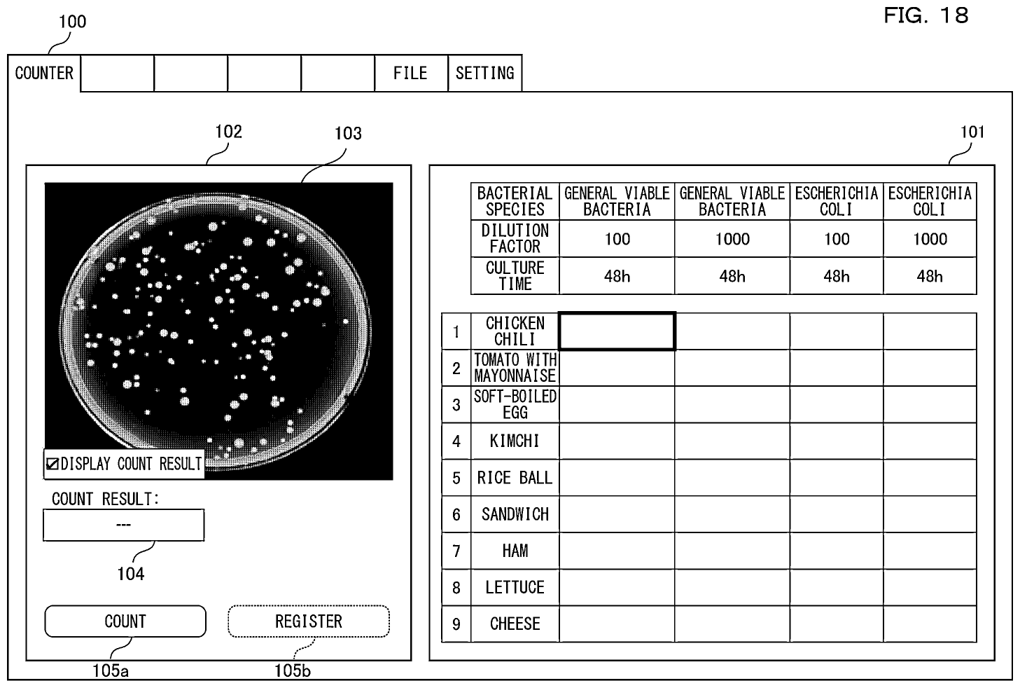

FIG. 18 illustrates the UI 100 displayed on the display device 37 by the MCU 30 when the first software button 105a or the first hardware button 8a, which is the capture button, is pressed. The image 103 (still image) of the Petri dish 15 is displayed in the result area 102. The MCU 30 assigns the first software button 105a from the capture button to a button (count button) for instructing a count.

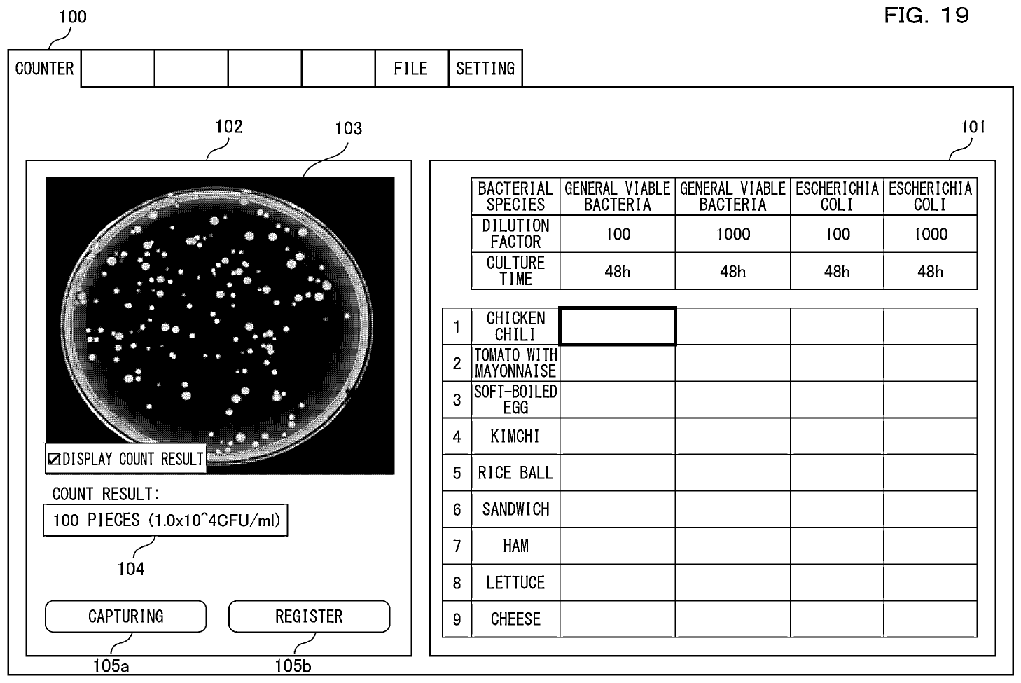

FIG. 19 illustrates the UI 100 displayed on the display device 37 by the MCU 30 when the first software button 105a or the first hardware button 8a, which is the count button, is pressed. When the count button is pressed, the MCU 30 instructs the head device 1a to count colonies. The MCU 20 of the head device 1a counts colonies in response to a count instruction and transmits a count value to the PC 1b. Note that the count processing may be executed by the MCU 30. The MCU 30 displays the count value in the count value area 104. At this time, the MCU 30 may convert the count value into CFU/mL (the number of colonies per section volume (milliliter)) and display CFU/mL in the count value area 104. For example, every time the count value area 104 is clicked with the pointer 57, the MCU 30 may switch display in order of only the count value, only CFU/mL, and the count value+CFU/mL. Note that CFU is an abbreviation for colony forming unit. Further, when the count value is acquired, the MCU 30 re-assigns the first software button 105a from the count button to the capture button. Moreover, the MCU 30 changes the second software button 105b and the second hardware button 8b assigned to the register button from an inoperable state to an operable state.

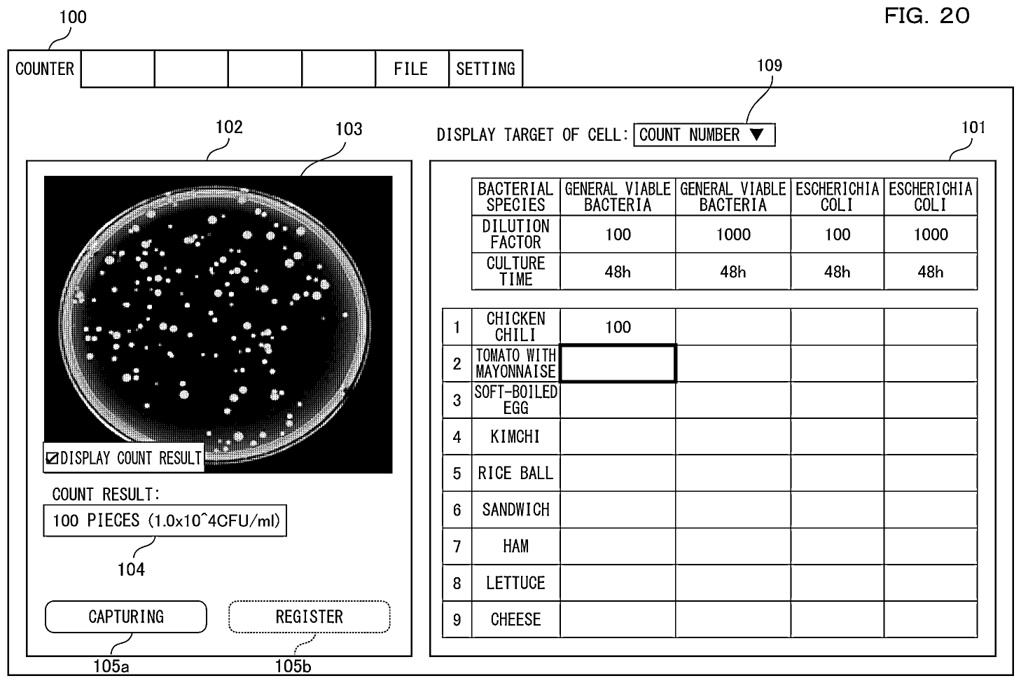

FIG. 20 illustrates a state in which the register button is pressed. The MCU 30 may write a count value to a currently selected cell and change the next cell to a state of being selected (cell of interest). In this example, the cell of interest (active cell) is changed from the cell of the first row to the cell of the second row. In this manner, the MCU 30 automatically selects the next cell, whereby burden on the user is mitigated. Note that the MCU 30 returns the second software button 105b and the second hardware button 8b, assigned to the register button, from the operable state to the inoperable state.

The UI 100 illustrated in FIG. 20 includes a cell display target change menu 109. In FIG. 20, “100” is displayed in a cell since “Count number” is selected in the change menu 109.

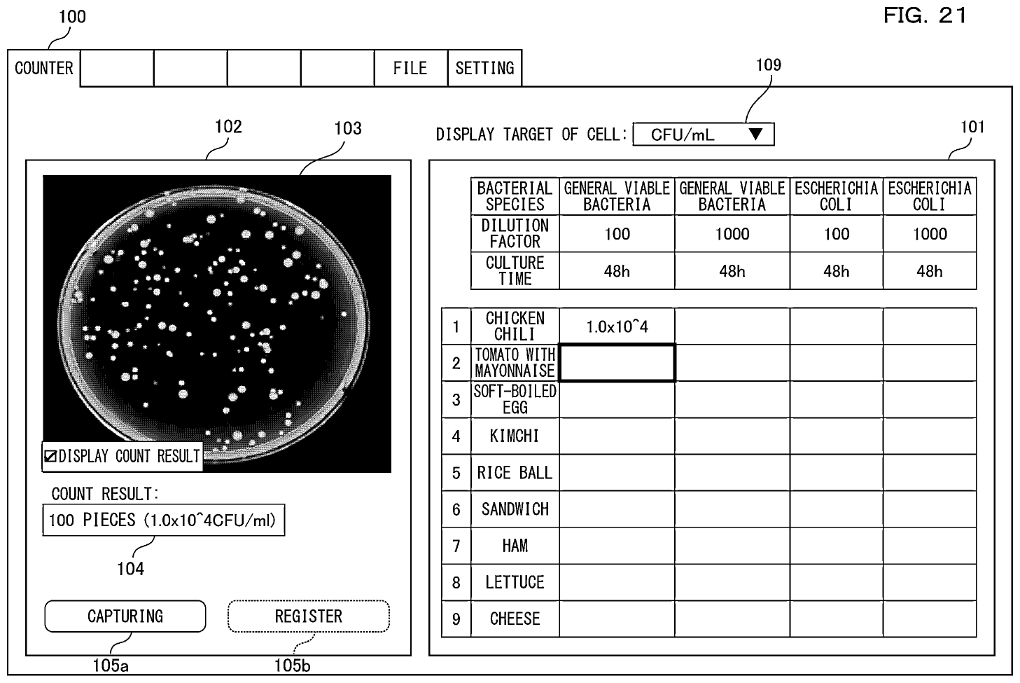

As illustrated in FIG. 21, when the user selects “CFU/mL” in the change menu 109, the MCU 30 changes a display target of the cell from “Count number” to “CFU/mL”. As a result, the user can easily switch the display target of the cell.

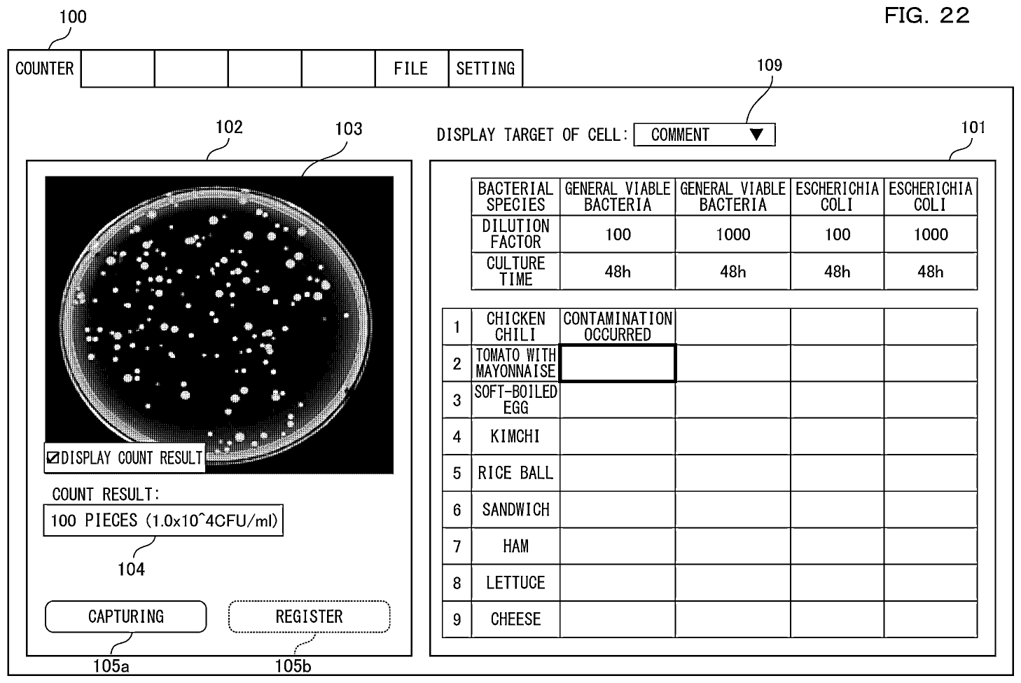

As illustrated in FIG. 22, when the user selects “Comment” in change menu 109, the MCU 30 changes the display target of the cell to “Comment”. As a result, the user can easily switch the display target of the cell to the comment. Further, in the UI 100, the user can also directly input a comment such as “Contamination occurs” as illustrated in FIG. 22. Here, the comment is a remark or the like included in one row of the count table.

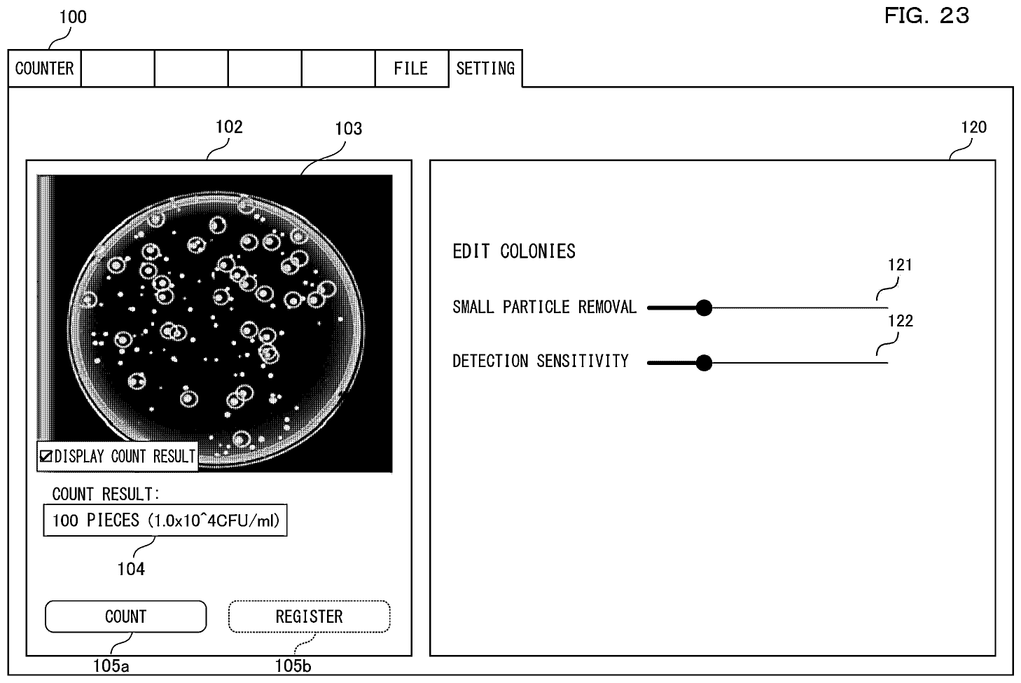

FIG. 23 illustrates the UI 100 displayed on the display device 37 by the MCU 30 when a cell to which a count value has been input is double-clicked. A setting screen 120 includes a control object for adjusting a parameter related to a colony detection algorithm out of a test condition associated with the cell selected by the double click. A slide bar 121 is, for example, a control object for setting a threshold to remove small particles by image processing. A slide bar 122 is a control object for adjusting colony detection sensitivity.

The MCU 30 may displays a mark such as a circle to be superimposed on a portion detected as a colony in the image 103 displayed in the result area 102. Since the MCU 30 changes an algorithm according to the adjustment of each of the slide bars 121 and 122, positions and the number of the marks indicating the colonies also change. As a result, the user can easily find an appropriate adjustment amount.

### [Assignment of Button]

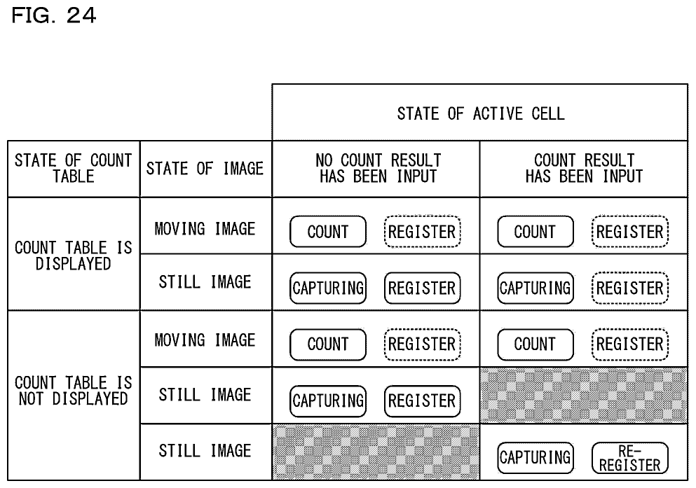

FIG. 24 illustrates an example of functions assigned to the first hardware button 8a (first software button 105a) and the second hardware button 8b (second software button 105b).

When a state of the count table is a state in which “Count table is displayed”, the image 103 is a moving image, and a state of the active cell indicates that no count value has been input, the count button is assigned to the first hardware button 8a (first software button 105a), and the register button (inoperable) is assigned to the second hardware button 8b (second software button 105b).

When a state of the count table is the state in which “Count table is displayed”, the image 103 is a moving image, and a state of the active cell indicates that a count value has been input, the count button is assigned to the first hardware button 8a (first software button 105a), and the register button (inoperable) is assigned to the second hardware button 8b (second software button 105b).

When a state of the count table is the state in which “Count table is displayed”, the image 103 is a still image, and a state of the active cell indicates that no count value has been input, the capture button is assigned to the first hardware button 8a (first software button 105a), and the register button (operable) is assigned to the second hardware button 8b (second software button 105b). Note that the capture button may be referred to as a re-capture button.

When a state of the count table is the state in which “Count table is displayed”, the image 103 is a still image, and a state of the active cell indicates that a count value has been input, the capture button is assigned to the first hardware button 8a (first software button 105a), and the register button (inoperable) is assigned to the second hardware button 8b (second software button 105b).

When a state of the count table is a state in which “Count table is not displayed”, the image 103 is a moving image, and a state of the active cell indicates that no count value has been input, the count button is assigned to the first hardware button 8a (first software button 105a), and the register button (inoperable) is assigned to the second hardware button 8b (second software button 105b).

When a state of the count table is the state in which “Count table is not displayed”, the image 103 is a moving image, and a state of the active cell indicates that a count value has been input, the count button is assigned to the first hardware button 8a (first software button 105a), and the register button (inoperable) is assigned to the second hardware button 8b (second software button 105b).

When a state of the count table is the state in which “Count table is not displayed”, the image 103 is a still image, and a state of the active cell indicates that no count value has been input, the capture button is assigned to the first hardware button 8a (first software button 105a), and the register button (operable) is assigned to the second hardware button 8b (second software button 105b).

When a state of the count table is the state in which “Count table is not displayed”, the image 103 is a still image, and a state of the active cell indicates that a count value has been input, the capture button is assigned to the first hardware button 8a (first software button 105a), and the re-register button (operable) is assigned to the second hardware button 8b (second software button 105b).

As illustrated in FIGS. 17 and 23, when a state of the count table is in the state in which “Count table is not displayed”, a UI that enables a change in a test condition is displayed instead of the count table. Therefore, the count value also changes when the test condition is changed, the MCU 30 inquires of a user whether or not to write (re-register) the changed count value to the active cell.

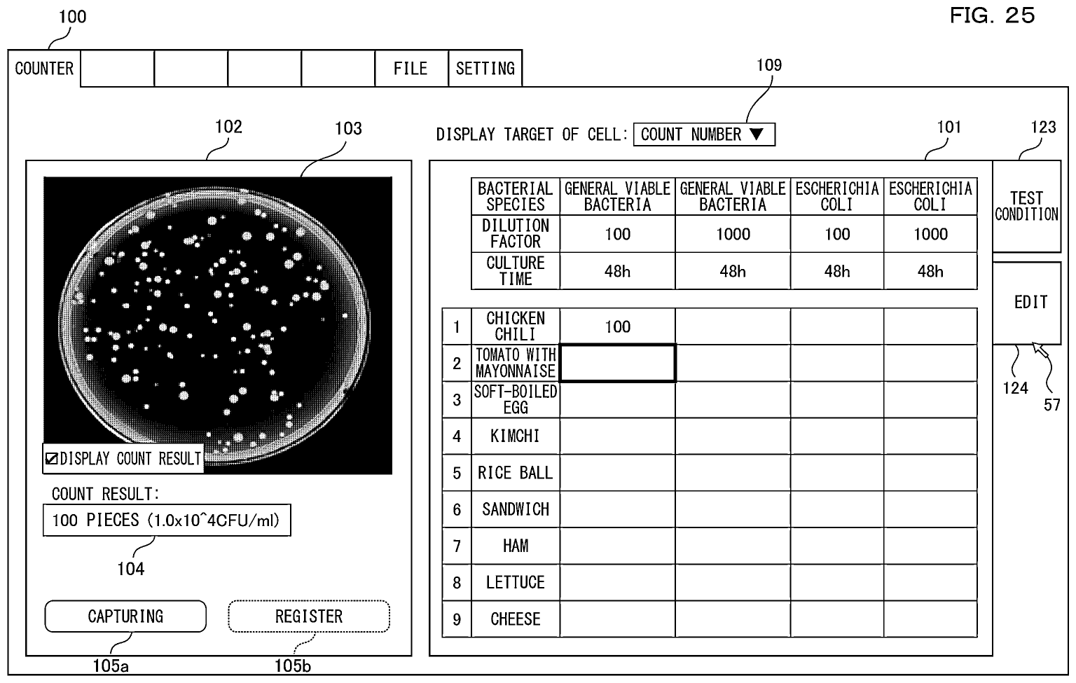

The state in which “Count table is not displayed” may be a state in which a count table to which a count result has been input is displayed and the count result can be re-edited. FIG. 25 illustrates the UI 100 with an edit tab 124 for re-editing. The state in which “Count table is not displayed” displayed in FIG. 24 may be a state in which a count table to which a count result has been input is displayed and the edit tab 124 has been clicked. In FIG. 25, a test condition tab 123 is a tab for displaying the confirmation screen 110 for a test condition illustrated in FIG. 17. As described above, the settings of the illumination and the main camera 11 can be changed on the confirmation screen 110.

When the count button is pressed while a moving image is being displayed in the result area 102 in this manner, the moving image is changed to a still image, the count result is displayed, and the register button is operable. When the register button is pressed while the still image is being displayed, the count result is written to the active cell, and the result area 102 returns to the state of displaying the moving image. When the count result that has been registered once is changed and the re-register button is pressed, the changed count result is overwritten on the count table, and the result area 102 returns to the state of displaying the moving image. When the capture button (re-capture button) is pressed while the result area 102 is displaying the still image, the result area 102 returns to the state of displaying the moving image.

### [Method for Identifying Count Table]

A user views a count table when culturing bacteria or counting colonies on the Petri dish 15. Here, there is a case where the date on which the count table has been created is different from the date on which preparation work and count work are executed while visually observing the count table. In this case, the user needs to read a desired count table from the storage device 35 and display the count table on the display device 37.

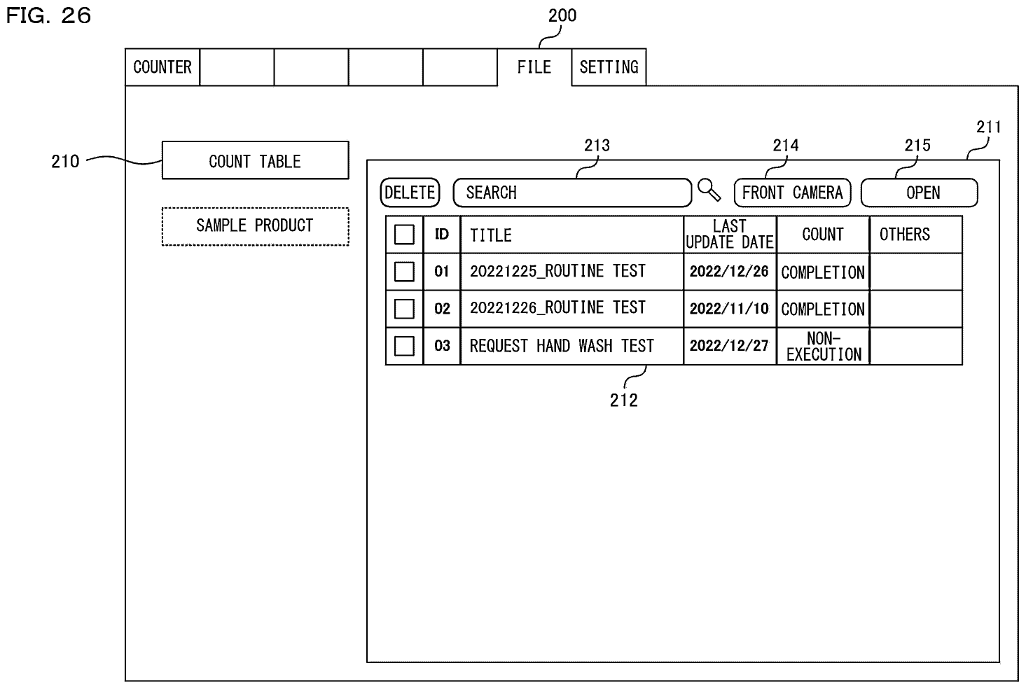

FIG. 26 illustrates a file UI 200 for calling a file such as a count table. A button 210 is a button for designating a count table as a calling target. A file list 211 is an area for displaying files stored in the storage device 35 as a list 212. When the button 210 is pressed, the MCU 30 displays only files having extensions specific to the count table among the plurality of files in the file list 211. The list 212 includes an ID (serial number), a title, a last update date, whether or not a count has been completed, and other information.

The search box 213 is a box to which a keyword for further searching for a desired file from the plurality of files included in the list 212 is input. A button 214 is a button for instructing activation of the front camera 10 in order to read an identification image given to a test list created by printing the count table on paper. An open button 215 is a button for instructing to open the count table selected from the list 212.

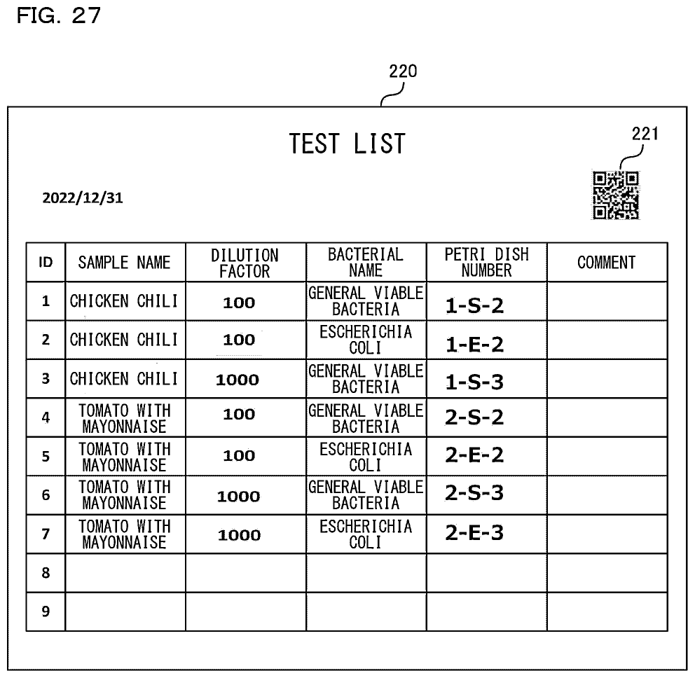

FIG. 27 illustrates a test list 220. The MCU 30 may output the test list 220 from the printer 38 or may output the test list to a display device of the terminal device 1c. The test list 220 is a list created from the corresponding count table 55. The order in which pieces of information are arrayed in the count table 55 and the order in which pieces of information are arrayed in the test list 220 may be different from each other or may coincide with each other. The test list 220 may be the same as the count table 55 displayed on the display device 37. Further, it is unnecessary to write a count result in the test list 220, and thus, a cell for writing the count result may be omitted. Moreover, in a case where the user uses the test list 220 for preparation of a culture sample, a culture time is not necessarily required. Therefore, a cell for the culture time may be omitted. In the test list 220, an identification image 221 such as a one-dimensional symbol or a two-dimensional symbol may be given as identification information for identifying each count table. The identification image 221 may include identification information of the user.

When the button 214 illustrated in FIG. 26 is pressed, the MCU 30 instructs the head device 1a to activate the front camera 10. The MCU 20 of the head device 1a activates the front camera 10 and attempts to read the identification image 221. The identification image 221 may be simply referred to as a symbol. At this time, the user aligns a position of the test list 220 with the front camera 10 such that the front camera 10 reads the identification image 221. When the MCU successfully reads identification image 221, the MCU 20 decodes the identification information from the identification image 221, and transmits the decoded identification information to the PC 1b. The MCU 30 reads a count table associated with the identification information received from the head device 1a from the storage device 35.

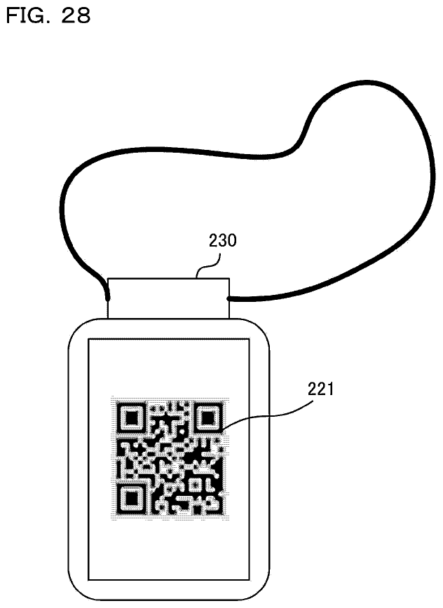

FIG. 28 illustrates a user authentication tag 230 worn by users. Due to data integrity rules, various restrictions are sometimes imposed on the users who use the head device 1a. For example, according to 21 CFR Part 11, which is a U.S. Food and Drug Administration (FDA) rule, users are required to be physically and logically managed. For example, the user authentication tag 230 may be used to limit available functions of the head device 1a and the PC 1b for each user. The MCU 20 or the MCU 30 may execute user authentication by causing the front camera 10 to read the identification image 221 of the user authentication tag 230. That is, the identification image 221 may include a symbol indicating account information such as a user name and a password. However, the user name and the password are encrypted, and then included the identification image 221.

As the restriction of functions for each user, the following is conceivable. As an example, the users are classified into an administrator, a leader, and an worker. The administrator can add a user and set authority of each of the users. The leader can create a count table, execute a count, store a count result, edit the count result, and output (print or transmit) the count result. The worker can execute a count and store a count result. The MCU 20 and the MCU 30 may restrict functions that can be executed by a user according to the authority of the user identified from the identification image 221.

Although the identification image 221 is read by the front camera 10 here, the identification image 221 may be read by the main camera 11.

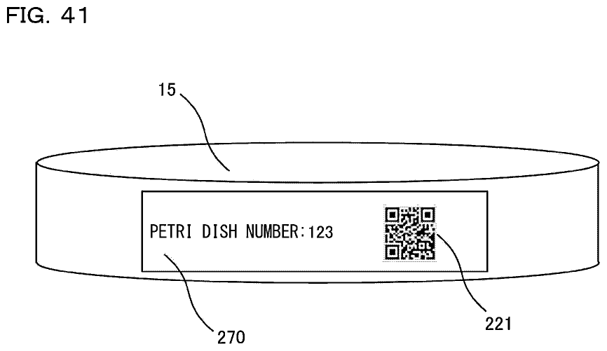

As illustrated in FIG. 41, the front camera 10 and the main camera 11 may capture and read an image of a Petri dish number or the identification image 221 printed on a seal 270 attached to the Petri dish 15. In the identification image 221, identification information of the count table 55, a sample name, unique identification information indicating a cell in which a count result is stored, and the like may be encoded. That is, by reading the identification image 221, the MCU 30 can identify the count table 55, the sample name, the Petri dish 15, and a test condition. Moreover, the MCU 30 transmits the identified test condition to the head device 1a, and the MCU 20 of the head device 1a can control the main camera 11, the ring illumination devices 12 and 13, and the coaxial illumination device 14 according to the received test condition to acquire an image.

### [Another Application Example of Front Camera]

### (1) Text Input to Cell

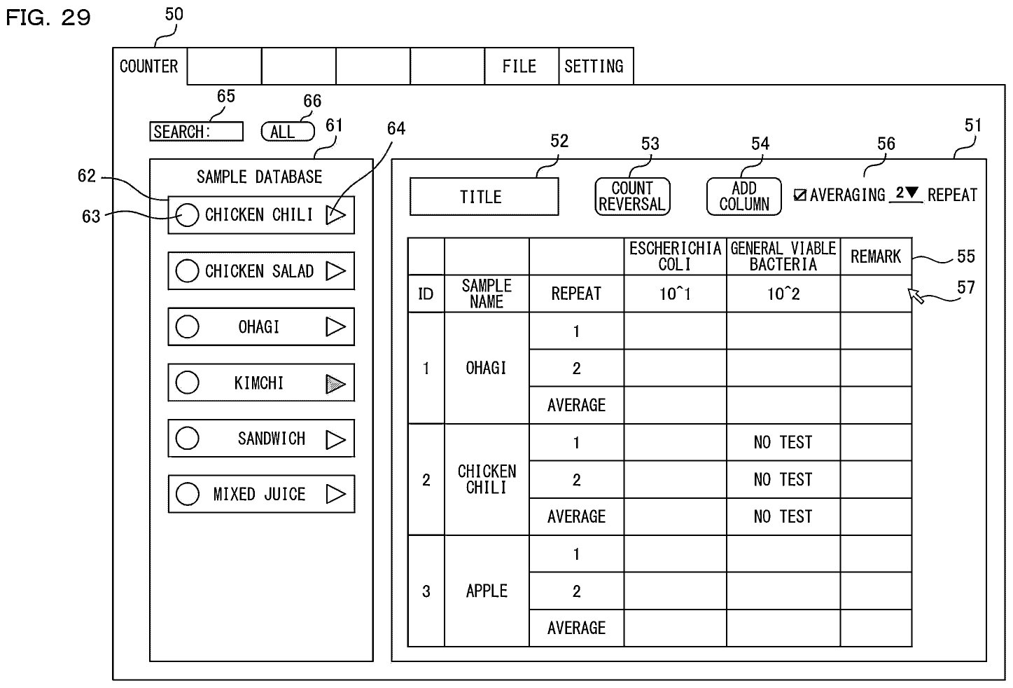

FIG. 29 illustrates the count table 55 in which a new column used as a remark has been added by pressing the button 54. The new column is defined as a free column, and can hold not only characters and numbers but also images and the like.

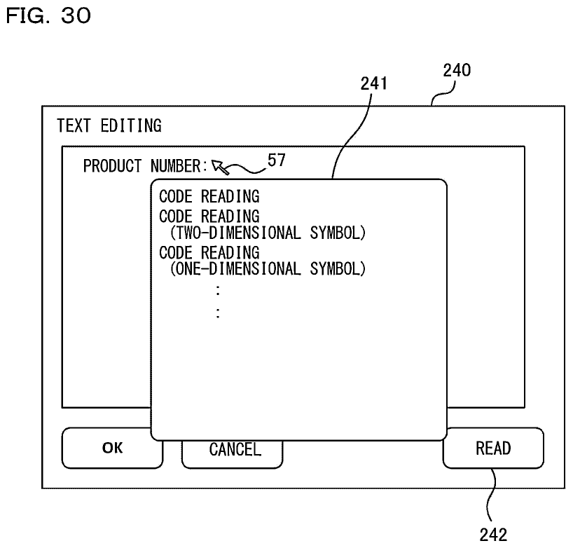

FIG. 30 illustrates an editing screen 240 displayed when a cell in the free column is right-clicked with the pointer 57. In this example, text “Product number:” for the cell has been input, and a dialog 241 for inputting the following text is displayed. The dialog 241 is displayed by right-clicking the inside of a text box of the editing screen 240 with the pointer 57. The dialog 241 includes a plurality of options for reading a code from a product or the like by using the front camera 10 and inputting a read result to the cell. In this example, the options include code reading by the front camera 10. For example, when “Code reading (one-dimensional symbol)” is selected by the pointer 57 and a read button 242 is pressed, the MCU 30 instructs the head device 1a to read a one-dimensional symbol.

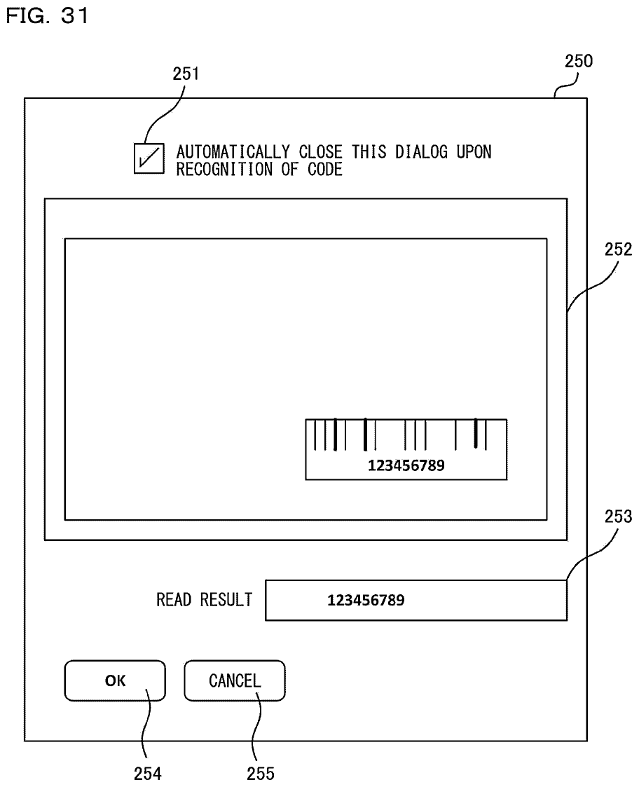

FIG. 31 illustrates a reading screen 250 displayed on the display device 37 when the read button 242 is pressed. A check box 251 is a check box for instructing that the MCU 20 automatically closes the reading screen 250 when the MCU 30 successfully recognizes a code. An image area 252 displays a moving image or a still image acquired by the front camera 10. A read result area 253 is an area for displaying text decoded from a one-dimensional symbol or a two-dimensional symbol.

The MCU 20 of the head device 1a activates the front camera 10, reads a one-dimensional code, decodes text from the one-dimensional code, and transmits the decoded text to the PC 1b. The user confirms the text displayed in the read result area 253, and presses an OK button 254 or a cancel button 255. When the OK button 254 is pressed, the MCU 30 closes the reading screen 250, returns to the editing screen 240, and inserts the text received from the head device 1a into the cell. When the cancel button 255 is pressed, the text received from the head device 1a is discarded, the reading screen 250 is closed to return to the editing screen 240.

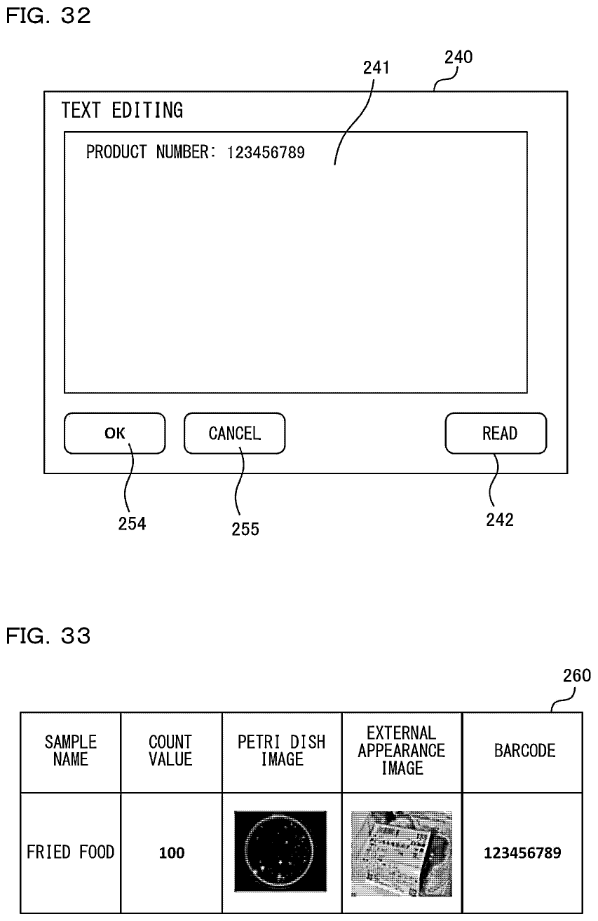

FIG. 32 illustrates the editing screen 240 into which text has been inserted. Here, when the OK button 254 is pressed, the MCU 30 closes the editing screen 240 and displays the UI 50 illustrated in FIG. 29 on the display device 37.

### (2) Image Input to Cell

FIG. 33 illustrates an example of a count report 260 created from a part of the count table or the count table. Image data can be associated with cells of the free column included in the count table 55. The MCU 30 controls the main camera 11 of the head device 1a to acquire an image (Petri dish image) of the Petri dish 15, and associates the acquired Petri dish image with a cell. Alternatively, the MCU 30 may control the front camera 10 of the head device 1a to acquire an image (Petri dish image) of the Petri dish 15, and associate the acquired Petri dish image with a cell. Similarly, the MCU 30 may control the front camera 10 of the head device 1a to acquire an external appearance image of a product, and associate the acquired external appearance image with a cell. Moreover, the MCU 30 may control the front camera 10 of the head device 1a to read and decode a barcode given to the product, and input the decoded product number and the like to cells. The MCU 30 may control the printer 38 to print the count report 260 on paper.

Although various images are acquired by the front camera 10 here, various images may be acquired by the main camera 11 and associated with cells.

### [Flowchart]

(1) Main processing of PC 1b

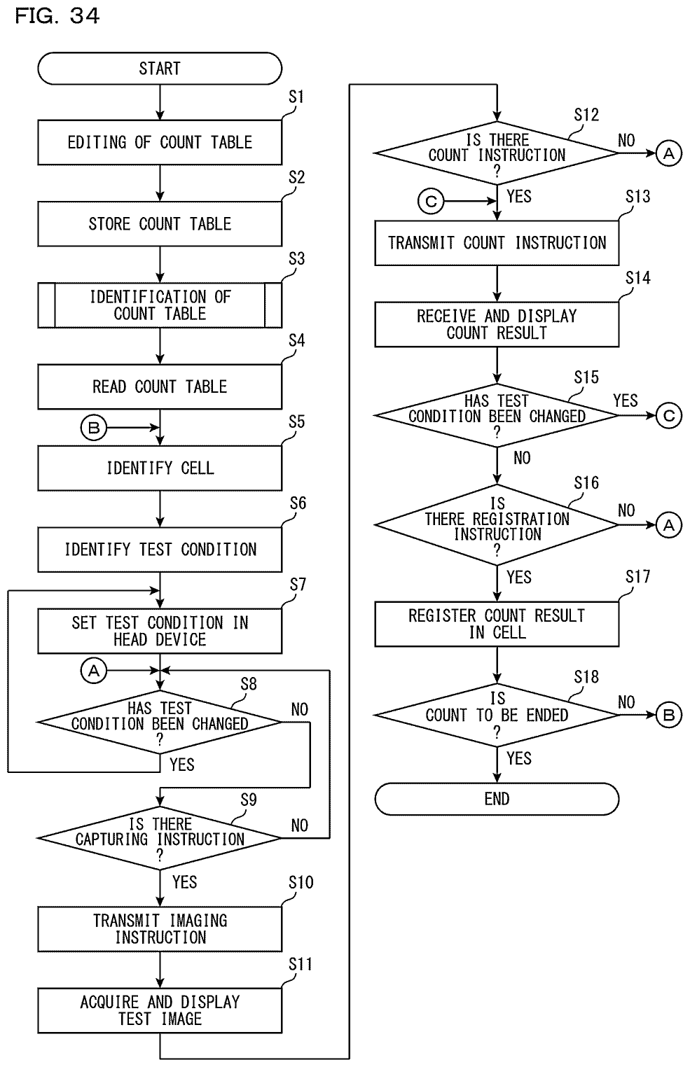

FIG. 34 is a flowchart illustrating a series of processes executed by the MCU 30 of the PC 1b. The MCU 30 executes the following processing according to the count application program stored in the storage device 35.

In S1, the MCU 30 executes editing of a count table. As described with reference to FIGS. 5 to 15 and the like, the count table is edited or created through the UI 50 and the like.

In S2, the MCU 30 stores the count table in the storage device 35.

In S3, the MCU 30 identifies the count table. The count table may be identified by using the front camera 10 and the test list 220 or the user authentication tag 230, or may be identified by using the file UI 200 illustrated in FIG. 26.

In S4, the MCU 30 reads the identified count table from the storage device 35. As a result, the UI 100 illustrated in FIG. 16 is displayed on the display device 37.

In S5, the MCU 30 identifies a cell to which a count value is to be written. First, a cell in the uppermost row in the count table may be selected, or a cell clicked by the pointer 57 may be selected.

In S6, the MCU 30 identifies a test condition associated with the active cell. For example, the MCU 30 reads the test condition associated with each cell from the storage device 35 when the count table has been created.

In S7, the MCU 30 sets the test condition associated with the active cell in the head device 1a. As described above, the sensitivity of the main camera 11, an illumination device to be turned on, brightness, the number of light emitting elements to be turned on (irradiation direction), image processing (HDR or ring removal), a count algorithm (a parameter such as a threshold), and the like are transmitted to the head device 1a.

In S8, the MCU 30 determines whether or not the test condition has been changed. As described above, the test condition associated with the cell can be changed at any time even during a test. Therefore, when the test condition is changed, the MCU 30 returns to S7 and transmits the changed test condition to the head device 1a. When the test condition is not changed, the MCU 30 proceeds to S9.

In S9, the MCU 30 determines whether or not a capturing instruction has been input by a user. The user can instruct capturing by pressing the first hardware button 8a of the head device 1a or the first software button 105a of the UI 100. When the capturing instruction is not input, the MCU 30 returns from S9 to S8. When the capturing instruction is input, the MCU 30 proceeds from S9 to S10.

In S10, the MCU 30 transmits an imaging instruction to the head device 1a.

In S11, the MCU 30 acquires an image (test image) of the Petri dish image 103 acquired by the main camera 11 from the head device 1a, and displays the test image in the result area 102 of the UI 100.

In S12, the MCU 30 determines whether or not a count instruction has been input. The user can input the count instruction by pressing the first hardware button 8a of the head device 1a or the first software button 105a of the UI 100 assigned as the count button. When the count instruction is not input, the MCU 30 returns from S12 to S8. When the count instruction is input, the MCU 30 proceeds from S12 to S13.

In S13, the MCU 30 transmits the count instruction to the head device 1a. Note that the MCU 30 performs count processing instead of the MCU 20 in a case where the count processing is performed by the PC 1b.

In S14, the MCU 30 receives a count result from the head device 1a, and displays the count result in the count value area 104. Note that, in a case where the MCU 30 executes the count processing in S14, the MCU 30 displays the counting result obtained by executing the count processing in the count value area 104.

In S15, the MCU 30 determines whether or not the test condition such as the image processing and the count algorithm has been changed. When the test condition is changed, the MCU 30 returns to S13. When the test condition is not changed, the MCU 30 proceeds to S16. Note that the change in the test condition in S8 is assumed to be a change in the test condition that requires re-acquisition of an image. The change in the test condition in S15 causes a change in image processing on the acquired image, but it is assumed that re-acquisition of an image is unnecessary.

In S16, the MCU 30 determines whether or not a registration instruction has been input by the user. The user can input the registration instruction by pressing the second hardware button 8b of the head device 1a or the second software button 105b of the UI 100 assigned as the register button. When the registration instruction has not been input, the MCU 30 returns from S16 to S8 to execute re-capturing or change the test condition. When the registration instruction is input, the MCU 30 proceeds from S16 to S17.

In S17, the MCU 30 registers the count result to the active cell.

In S18, the MCU 30 determines whether or not all counts have been ended. For example, when the count results have been input to all the cells existing in the count table, the MCU 30 determines that the counts have been ended. When there is still a cell without any input, the MCU 30 proceeds from S18 to S5, and changes the active cell to the next cell (cell identification).

### (2) Main Processing of Head Device 1a

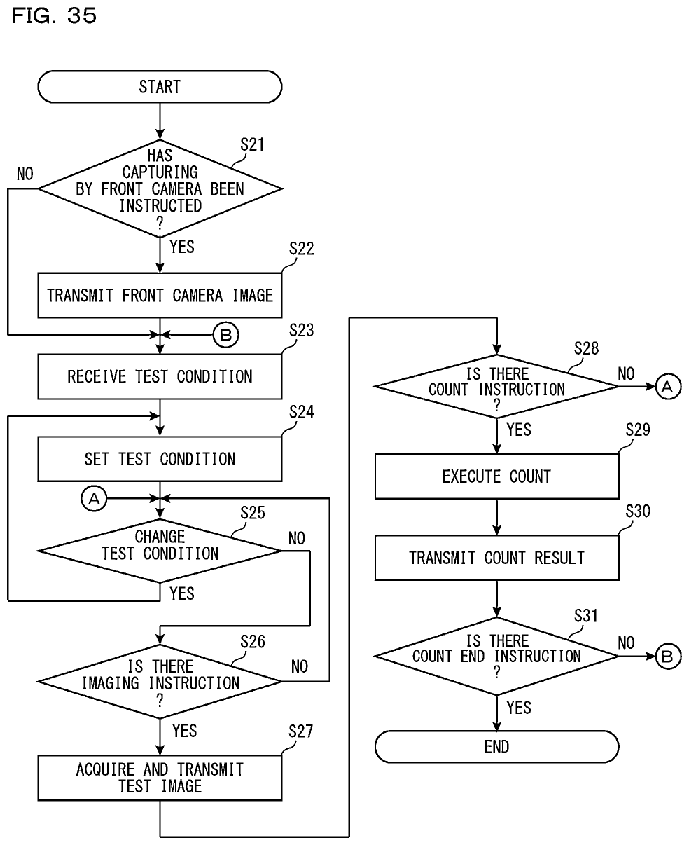

FIG. 35 illustrates a series of processes executed by the MCU 20 of the head device 1a according to the control program.

In S21, the MCU 20 determines whether or not a capturing instruction with respect to the front camera 10 has been received. In a case where the identification image 221 of the test list 220 is read as described above, the capturing instruction (a code reading instruction) to the front camera 10 is input from the PC 1b to the head device 1a. When the capturing instruction to front camera 10 has not been input, the MCU 20 proceeds from S21 to S23. When the capturing instruction to the front camera 10 is input, the MCU 20 proceeds from S21 to S22.

In S22, the MCU 20 activates the front camera 10 to acquire an image (front camera image), and transmits the front camera image or a decoding result of a symbol to the PC 1b.

In S23, the MCU 20 receives a test condition from the PC 1b and stores the test condition in the storage device 25.

In S24, the MCU 20 sets the test condition for each section. The sensitivity out of the test condition is set in the imaging control section 21. An illumination device to be turned on, brightness, an illumination direction, and the like are set in the illumination control section 22.

In S25, the MCU 20 determines whether or not a change instruction for the test condition has been received from the PC 1b. The change instruction is received together with a new test condition. When the change instruction for the test condition is received, the MCU 20 returns to S24 and sets the new test condition. When the change instruction has not been received, the MCU 20 proceeds from S25 to S26.

In S26, the MCU 20 determines whether or not an imaging instruction has been received from the PC 1b. When the imaging instruction has not been input, the MCU 20 returns from S26 to S25. When the imaging instruction is input, the MCU 20 proceeds from S26 to S27.

In S27, the MCU 20 activates the main camera 11, acquires a test image, and transmits the test image to the PC 1b.

In S28, the MCU 20 determines whether or not a count instruction has been input from the head device 1a. When the count instruction has not been input, the MCU 20 returns from S28 to S25. When the count instruction is input, the MCU 20 proceeds from S28 to S29.

In S29, the MCU 20 executes a count of colonies according to the test condition.

In S30, the MCU 20 transmits a count result to the PC 1b.

In S31, the MCU 20 determines whether or not a count end instruction has been received. When the count end instruction is received, the MCU 20 ends the count. When the count end instruction has not been received, the MCU 20 returns from S31 to S23, and receives a test condition for the next cell.

### (3) Registration of Sample Database

A count table has a plurality of rows and columns, and each cell is associated with a test condition. The count table and a test list may be created again for each day. Meanwhile, there is also a case where a test is executed for the same sample every day. Therefore, burden of count table creation processing is mitigated when a count table is registered in the sample DB 40 in advance for a sample with a high test frequency. Therefore, when a sample table has been created, the user may register a row element corresponding to each sample in the sample DB 40.

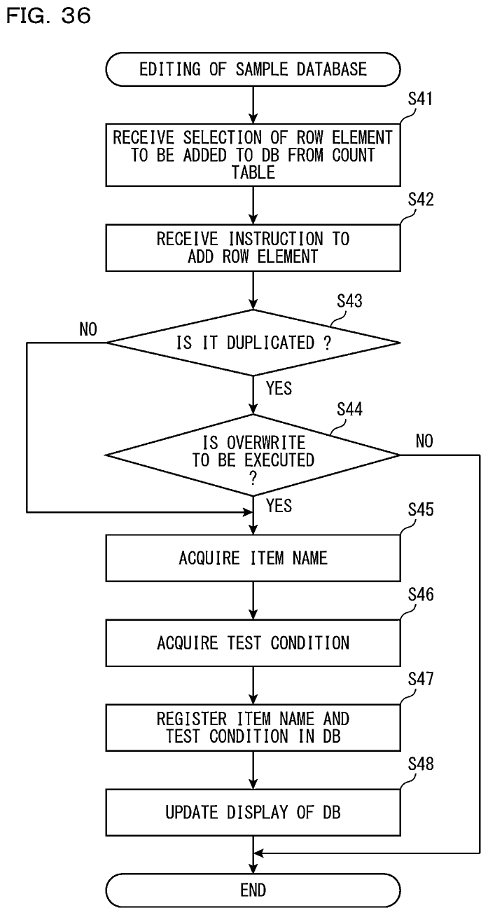

FIG. 36 is a flowchart illustrating editing processing of the sample DB 40 executed by the MCU 30 of the PC 1b. The MCU 30 executes the following processing according to the application program 39 stored in the storage device 35.

In S41, the MCU 30 receives selection of a row element to be registered in the sample DB 40 among a plurality of row elements included in the count table. For example, the MCU 30 may receive a click by the pointer 57 on any row element among the row elements included in the sample table.

In S42, the MCU 30 receives an addition instruction for the selected row element. For example, the addition instruction may be input when a right click is executed by the pointer 57 in a state in which the row element has been selected.

In S43, the MCU 30 acquires a sample name of the row element instructed to be added, and determines whether or not the same sample name has already been registered in the sample DB 40 (duplication determination). When the row element instructed to be added does not already exist, the MCU 30 proceeds from S43 to S45. When the row element instructed to be added exists in the sample DB 40, the MCU 30 proceeds from S43 to S44.

In S44, the MCU 30 inquires of the user whether or not to overwrite the row element in the sample DB 40. When a cancellation instruction is input, the MCU 30 cancels the addition of the row element. When an overwriting instruction is input, the MCU 30 proceeds from S44 to S45.

In S45, the MCU 30 acquires an item name (for example, a sample name, a bacterial species, a culture medium type, or a dilution factor) constituting the row element to be added.

In S46, the MCU 30 acquires a test condition associated with a cell of the row element from the storage device 35.

In S47, the MCU 30 registers the item name and the test condition in the sample DB 40.

In S48, the MCU 30 updates display of the sample DB 40 in the UI 50.

### (4) Editing of Count Table

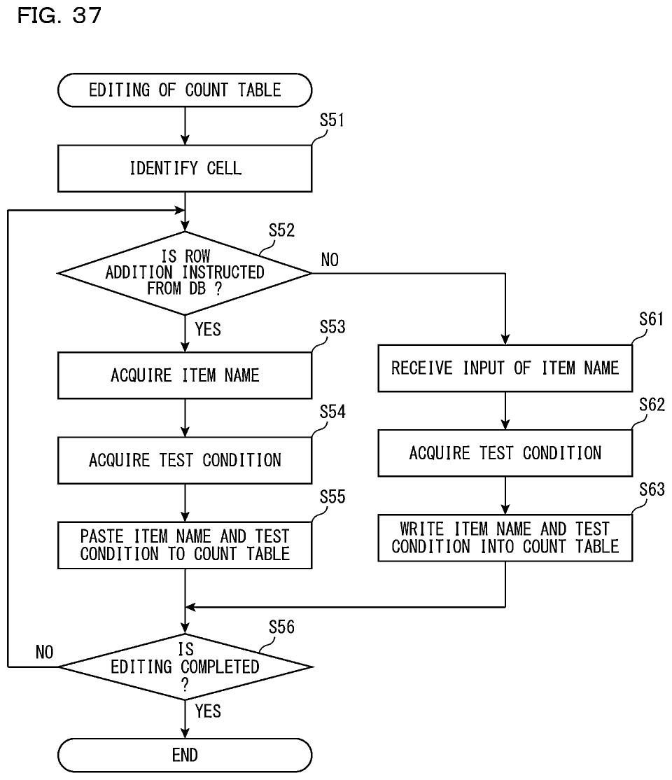

FIG. 37 is a flowchart illustrating count table editing processing executed by the MCU 30 of the PC 1b. The MCU 30 executes the following processing according to the count application program stored in the storage device 35.

In S51, the MCU 30 identifies a position (cells or a row) to which a row element is to be newly added in a count table. For example, the MCU 30 selects a row next to the last row in which a sample name has been input in the count table. Note that a new row may be selected between a row and another row. For example, when a row in which a sample name has been input in the count table is selected and right-clicked by the pointer 57, an empty row is added next to the selected row.

In S52, the MCU 30 determines whether or not an instruction to add a row from the sample DB 40 has been issued. For example, when the button 64 of the UI 50 is pressed, the MCU 30 recognizes that the instruction to add a row from the sample DB 40 has been issued. When the instruction to add a row from the sample DB 40 is issued, the MCU 30 proceeds to S53. When the instruction to add a row from the sample DB 40 has not been issued, the MCU 30 proceeds to S61.

In S53, the MCU 30 acquires an item name of the row instructed to be added from the sample DB 40.

In S54, the MCU 30 acquires a test condition of the row instructed to be added from the sample DB 40.

In S55, the MCU 30 attaches the acquired item name and test condition to the count table. That is, the MCU 30 adds a new row element to the count table. Note that the MCU 30 may determine whether or not a row element designated by the user among the plurality of row elements held in the sample DB 40 is included in a new count table. Moreover, when it is determined that the row element designated by the user is not included in the new count table, the MCU 30 may determine whether or not the row element designated by the user includes cells of a column element not included in the new count table. When it is determined that the row element designated by the user includes the cells of the column element not included in the new count table, the MCU 30 adds the column element to the new count table. That is, the column element is also added to the row element given with another sample name already existing in the count table.

In S56, the MCU 30 determines whether or not to complete editing. When the user instructs to complete editing, the MCU 30 stores the count table in the storage device 35. When the user does not instruct to complete editing, the MCU 30 returns from S56 to S51.

When a new row is added without using the sample DB 40, the MCU 30 receives an input of an item name through the keyboard 32 or the pointing device 33 in S61.

In S62, the MCU 30 receives an input of a test condition through the keyboard 32 or the pointing device 33.

In S63, the MCU 30 writes the acquired item name and test condition in the count table. Thereafter, the MCU 30 proceeds to S56.

### (5) Identification of Count Table

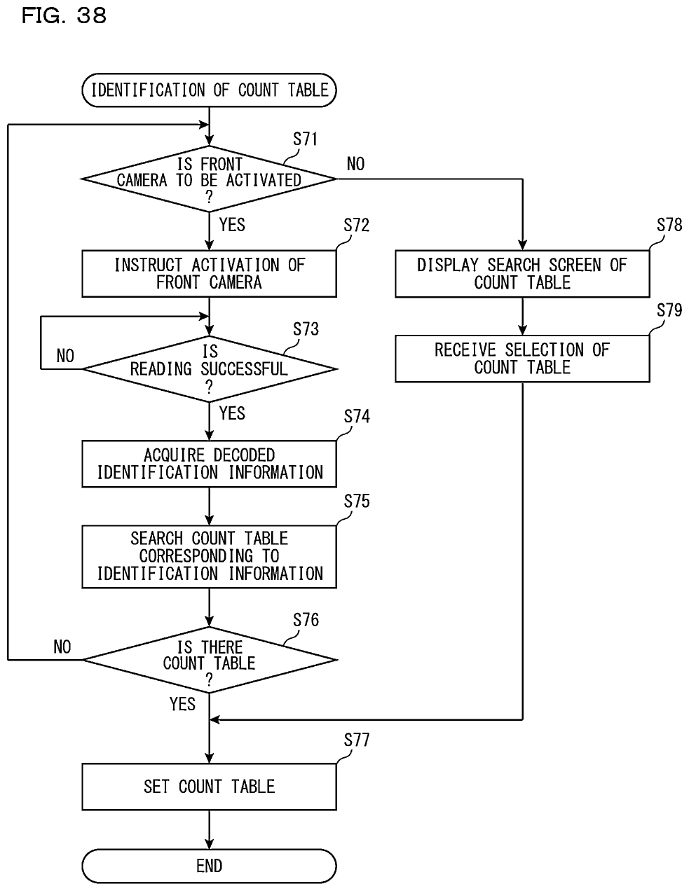

FIG. 38 is a flowchart illustrating count table identification processing executed by the MCU 30 of the PC 1b. The MCU 30 executes the following processing according to the count application program stored in the storage device 35.

In S71, the MCU 30 determines whether or not an activation instruction for the front camera 10 has been input. For example, when the button 214 of the file UI 200 illustrated in FIG. 26 is clicked, the MCU 30 determines that the activation instruction has been input, and proceeds to S72.

In S72, the MCU 30 transmits the activation instruction for the front camera 10 to the head device 1a.

In S73, the MCU 30 waits for the head device 1a to successfully read the identification image 221.

In S74, the MCU 30 acquires identification information decoded from the identification image 221 in the head device 1a.

In S75, the MCU 30 searches the storage device 35 for a count table corresponding to the identification information.

In S76, the MCU 30 determines whether or not the count table corresponding to the identification information has been found. In a case where the count table does not exist, the MCU 30 returns to S71. In a case where the count table exists, the MCU 30 proceeds to S77.

In S77, the MCU 30 reads the count table from the storage device 35 and sets the count table in the UI 100.

When the activation instruction has not been input in S71, the MCU 30 proceeds to S78. In S78, the MCU 30 displays a count table search screen on the display device 37. In S79, the MCU 30 receives selection of a count table. For example, any count table may be selected in the file UI 200 illustrated in FIG. 26. Thereafter, the MCU 30 proceeds to S77.

### (6) Count of Colonies

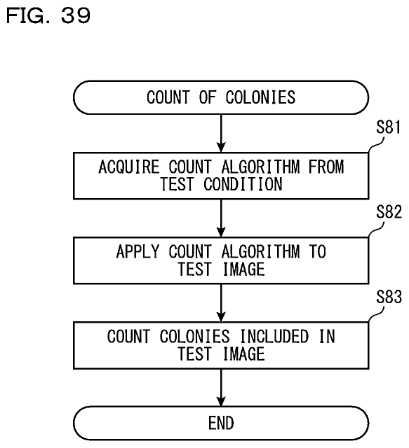

FIG. 39 illustrates colony count processing executed by the MCU 20 of the head device 1a according to the control program. However, image processing and count processing may be executed by the MCU 30.

In S81, the MCU 20 acquires a count algorithm from a test condition received from the PC 1b. Specifically, image processing and a threshold parameter (for example, a binarization threshold) used in the count algorithm are acquired.

In S82, the MCU 20 applies the count algorithm to a test image acquired by the main camera 11. For example, image processing such as HDR or ring removal is applied to the test image.

In S83, the MCU 20 counts colonies included in the test image according to the test condition (threshold parameter).

### (7) Registration of Information in Free Column (for Example, Remark Cell)

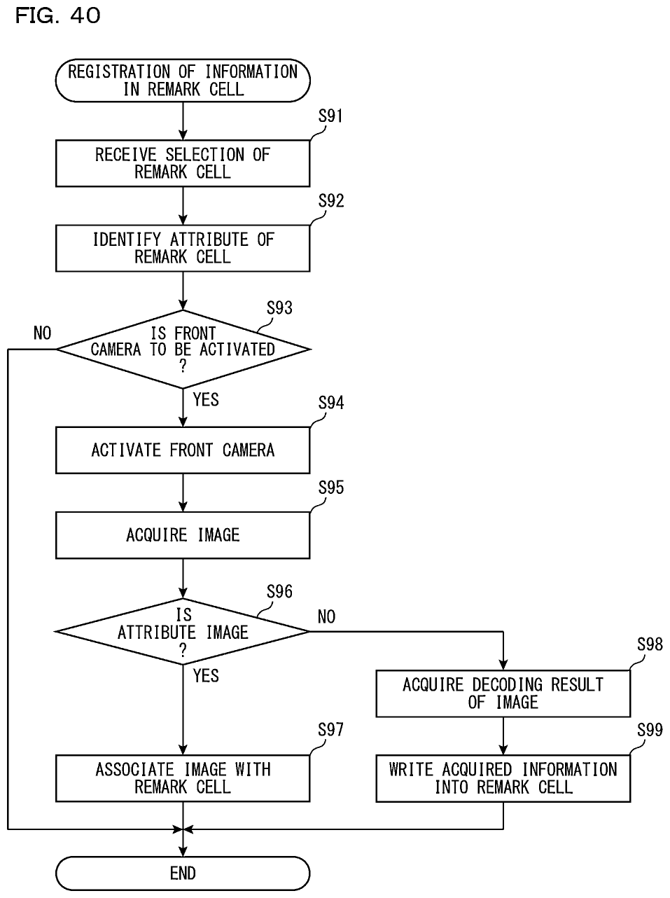

FIG. 40 is a flowchart illustrating information registration processing with respect to a free column, such as a remark cell, executed by the MCU 30 of the PC 1b. The MCU 30 executes the following processing according to the count application program stored in the storage device 35.

In S91, the MCU 30 receives selection of a remark cell. Although the remark cell is used as an example here, a cell of another free column may be used. The MCU 30 sets the remark cell selected by the pointer 57 as an active cell.

In S92, the MCU 30 identifies an attribute of the remark cell. An attribute (for example, a count value, a character string, or an image) may be given to each cell in advance. The MCU 30 reads the attribute of each cell from the storage device 35.

In S93, the MCU 30 determines whether or not activation of the front camera has been instructed. When the activation of the front camera 10 is not instructed, the MCU 30 inputs text input from the keyboard 32 or the like to the remark cell. On the other hand, when the activation instruction is input, the MCU 30 proceeds to S94.

In S94, the MCU 30 activates the front camera 10 of the head device 1a.

In S95, the MCU 30 acquires an image by the front camera 10.

In S96, the MCU 30 determines whether or not the identified attribute is an image. When the attribute is an image, the MCU 30 proceeds to S97.

In S97, the MCU 30 associates the image (for example, an external appearance image of a product) acquired by the front camera 10 with the remark cell.

When it is determined in S96 that the identified attribute is not an image, the MCU 30 proceeds to S98.

In S98, the MCU 30 acquires a decoding result of the image acquired by the front camera 10 from the head device 1a.

In S99, the MCU 30 writes the acquired information (the decoding result (for example, a serial number of the product)) into the remark cell.

### [Details of Coaxial Illumination Device]

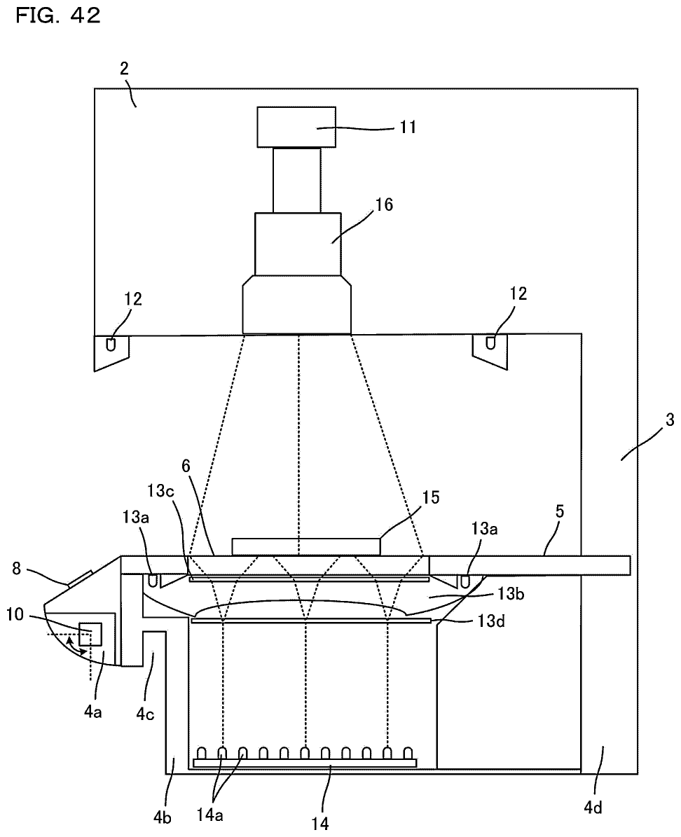

FIG. 42 is a schematic cross-sectional view of the head device 1a. Test light output from the plurality of light emitting elements 14a provided in the coaxial illumination device 14 is diffused by the diffusion plate 13d and the diffusion plate 13c, is incident from a back surface of a transmission window 6a, and is emitted from a front surface thereof, and illuminates a test individual accommodated in the Petri dish 15. The main camera 11 captures an image of the test individual illuminated by the test light from the coaxial illumination device 14 in this manner, and generates a test image. Since the diffusion plate 13d and the diffusion plate 13c are adopted, it is possible to achieve a high degree of diffusion with a simple configuration. The reflection plate 13b has a shape like a bowl or a dish without a bottom, and the diffusion plate 13d is provided at a portion corresponding to the bottom. The diffusion plate 13c is arranged on an upper surface or an exit surface of the bowl-shaped reflector 13b.

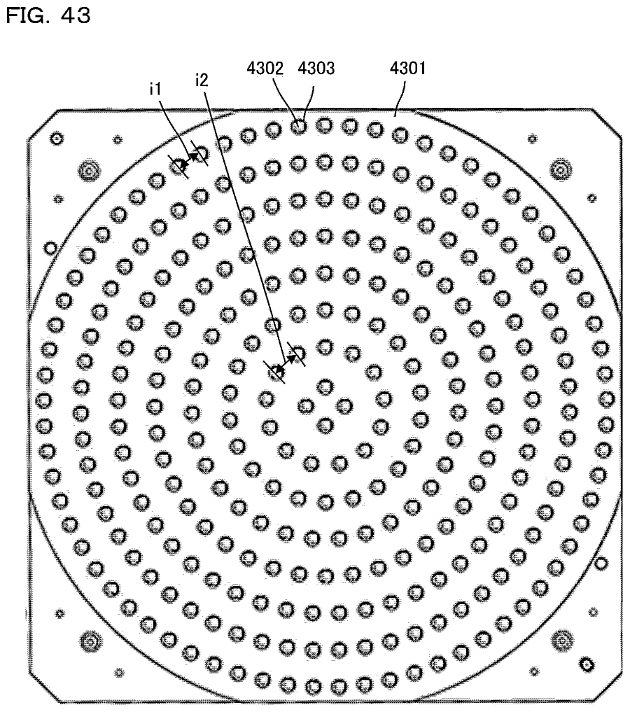

FIG. 43 is a plan view of the coaxial illumination device 14. A light guide 4302 having a substantially rectangular parallelepiped shape is provided above each of the plurality of light emitting elements 14a constituting the coaxial illumination device 14. That is, the light emitting elements 14a and the light guides 4302 are provided on a one-to-one basis. The ride guide 4302 guides light incident from the bottom surface side to the top surface side. A light distribution regulating plate 4301 is provided above the light guide 4302. The light distribution regulating plate 4301 is formed of a light shielding member made of metal such as aluminum or resin. The light distribution regulating plate 4301 is provided with a plurality of substantially circular apertures 4303. The light guides 4302 and the apertures 4303 are provided on a one-to-one basis. The light emitted from the ride guide 4302 is shaped by the aperture 5404.

As can be seen from FIG. 43, a plurality of light emitting elements 14a are arranged in each of a plurality of (for example, 8) concentric circles. Further, the density of a plurality of light emitting elements 14a arranged on an outer concentric circle is higher than the density of a plurality of light emitting elements 14a arranged on an inner concentric circle. That is, a distance (arrangement interval i1) between two light emitting elements adjacent to each other on the outer concentric circle is shorter than a distance (arrangement interval i2) between two light emitting elements adjacent to each other on the inner concentric circle. This helps to make the test light output from the coaxial illumination device 14 uniform. That is, the amount of light of the test light near the center of the coaxial illumination device 14 and the amount of light of the test light near the outer edge of the coaxial illumination device 14 can be made uniform.

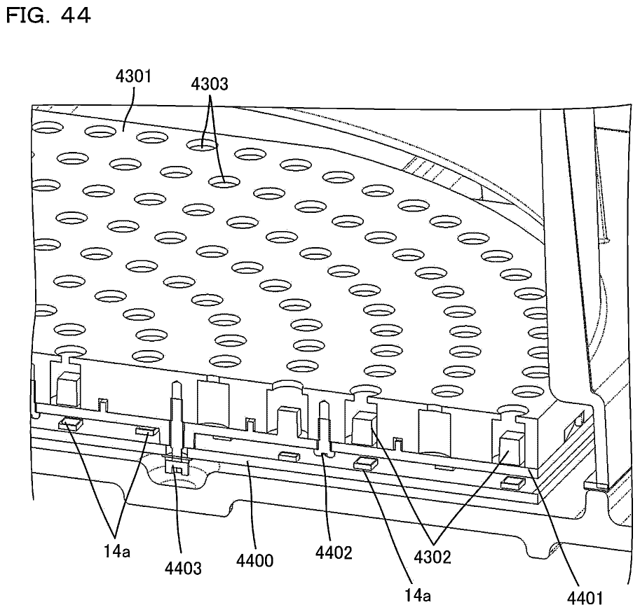

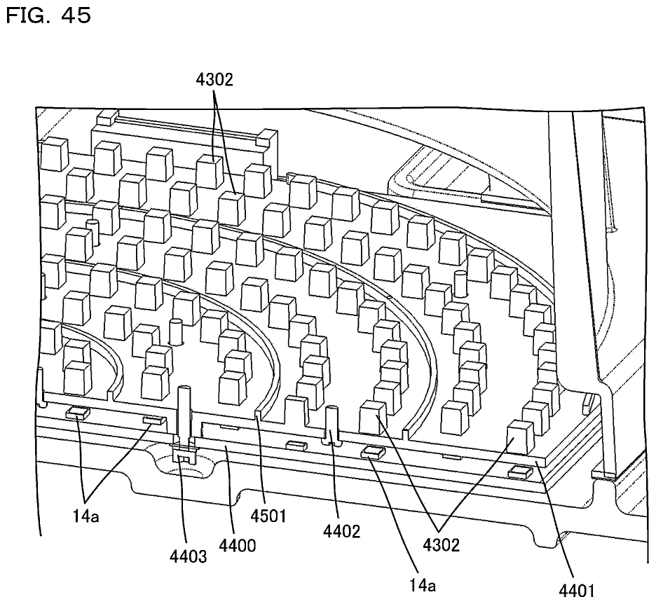

FIGS. 44 and 45 are cross-sectional perspective views of the light distribution regulating plate 4301, the light guides 4302, a light guide substrate 4401, the light emitting elements 14a, and a substrate 4400. The plurality of light emitting elements 14a are mounted on the substrate 4400. A screw 4402 is fastened to the light distribution regulating plate 4301 via the light guide substrate 4401 to fix the light guide substrate 4401 to the light distribution regulating plate 4301. A screw 4403 is fastened to the light distribution regulating plate 4301 via the substrate 4400 and the light guide substrate 4401 to fix the light guide substrate 4401 and the light distribution regulating plate 4301 to the substrate 4400. Note that the plurality of light guides 4302 may be integrated with the light guide substrate 4401. The plurality of light guides 4302 and the light guide substrate 4401 are molded using resin (for example, acrylic) or glass having translucency.

As illustrated in FIG. 45, the light guide substrate 4401 may have a plurality of protrusions 4501 arranged in a plurality of concentric circles. The plurality of protrusions 4501 help to maintain a shape of the light guide substrate 4401. That is, the plurality of protrusions 4501 help to increase the rigidity of the light guide substrate 4401.

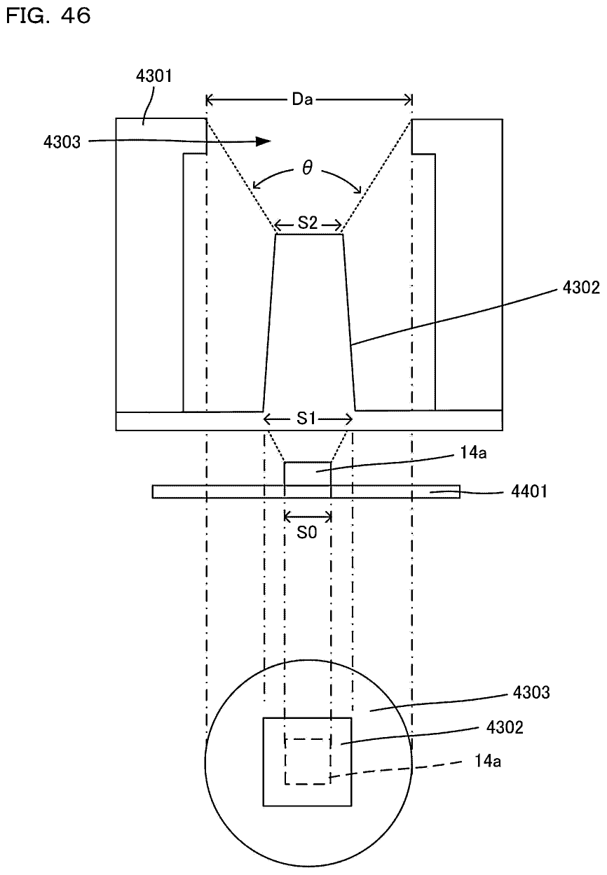

FIG. 46 is a schematic view for describing a light distribution angle of the coaxial illumination device 14. The light distribution angle of the coaxial illumination device 14 can be designed to be, for example, about 10 degrees or more and 25 degrees or less. When the light distribution angle is designed to have a value outside this range, it is difficult to capture an image of the test individual placed near the edge of the transmission window 6. Further, there is a possibility that a stain and a scratch of the Petri dish 15 are erroneously detected. In order to set the light distribution angle of the coaxial illumination device 14 to about 10 degrees or more and 25 degrees or less, it is sufficient to design a light distribution angle θ of a light beam emitted from each of the plurality of light emitting elements 14a to be 20 degrees or more and 50 degrees or less.

As illustrated in FIG. 46, a diameter of the aperture 4304 is denoted by Da. The light guide 4302 may be a rectangular parallelepiped or a quadrangular pyramid, but for convenience of description, the light guide 4302 is assumed to be a truncated cone in FIG. 46. In the light guide 4302, the area of an exit surface is denoted by S2, and the area of an incident surface is denoted by S1. The area of light emission of the light emitting element 14a is denoted by S0. In order to set a light distribution angle of one set of the light emitting element 14a, the light guide 4302, and the aperture 4304 to a desired angle, it is sufficient to satisfy the following conditional expressions based on the etendue conservation law.

$$0.8 \text{=<} S 1 / S 2 \text{=<} 1.2 \qquad ( 1 )$$

$$1. \text{=<} S 2 / S 9 \text{=<} 25 \qquad ( 2 )$$

$$Dp \text{=<} Da \qquad ( 3 )$$

Here, Dp denotes a diameter of the exit surface of the light guide 4302. The diameter of the aperture 4303 is denoted by Da.

In general, when the light distribution angle θ is less than 20 degrees, the test image is likely to be affected by dirt, a scratch, and the edge of the transmission window 6, which leads to an erroneous count value of colonies. On the other hand, when the light distribution angle θ exceeds 50 degrees, the degree of diffusion becomes too high, and the contrast of the test image becomes low.

Note that the aperture 4303 can be omitted when Formulas (1) and (2) are satisfied. However, the aperture 4304 is provided in consideration of a manufacturing error and an attachment error of the light guide 4302 and the light emitting element 14a. Since the aperture 4304 is provided, the light distribution angle θ can be brought close to an ideal state.

In the present embodiment, an illumination optical system as one set of the light guide 4302 and the aperture 4304 is adopted, but another illumination optical system may be adopted. For example, a shell-type light emitting diode having the light distribution angle θ of 20 degrees or more and 50° C. or less can be replaced with one set of the light emitting element 14a, the light guide 4302, and the aperture 4304.

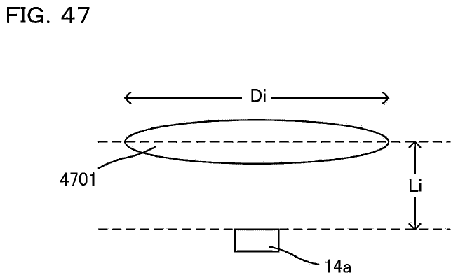

As illustrated in FIG. 47, the light guide 4302 and the aperture 4304 may be replaced with a lens 4701. In this case, a numerical aperture NA and a diameter Di of the lens 4701 are required to satisfy the following conditions, respectively.

$$NA >= 0.17 \qquad ( 4 )$$

$$Di / 2 Li >= 0.17 \qquad ( 5 )$$

Here, Li denotes a distance between the center of the lens 4701 and the exit surface of the light emitting element 14a. When Formulas (4) and (5) are satisfied, NA of the lens 4701 becomes 0.17 or more, an incident angle of light from the light emitting element 14a by the lens 4701 becomes NA0.17 or more, and the light distribution angle θ becomes 20 degrees or more.

### [Modified Example of Coaxial Illumination Device]

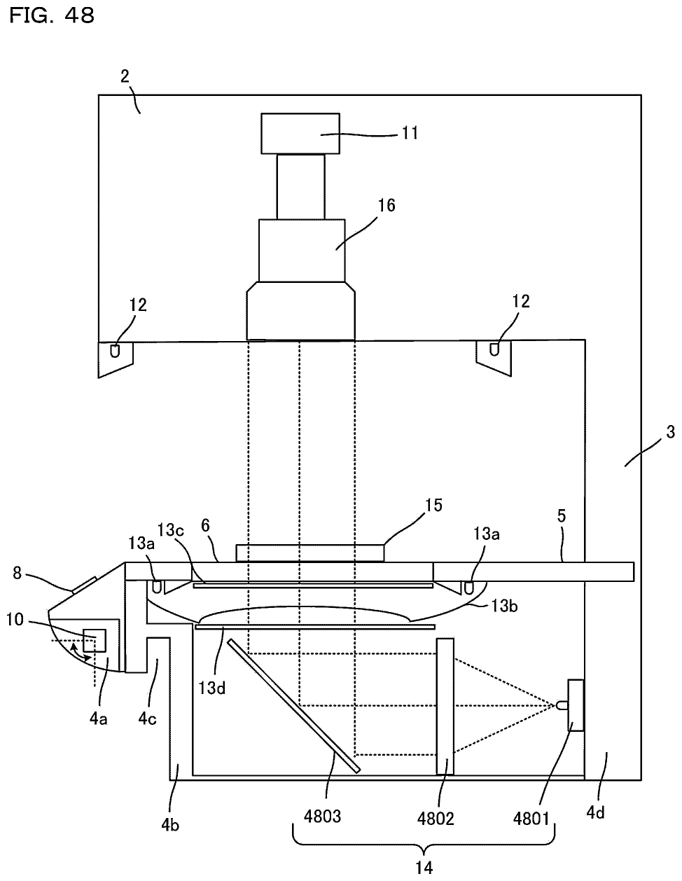

FIG. 48 illustrates the coaxial illumination device 14 adopting a telecentric optical system 4802. In the present embodiment, the coaxial illumination device 14 is required to be generally telecentric. Therefore, the coaxial illumination device 14 includes a light source 4801 such as a halogen light, the telecentric optical system 4802, and a mirror 4803. Test light output from the light source 4801 passes through the telecentric optical system 4802 to become a highly telecentric test light beam. The test light beam is deflected by the mirror 4803, passes through the diffusion plate 13d and the diffusion plate 13c, and illuminates a test individual in the Petri dish 15 from a back surface of the transmission window 6. The light source 4801 may be a surface light source.

Since the coaxial illumination device 14 illustrated in FIG. 48 requires the telecentric optical system 4802, the coaxial illumination device 14 becomes large. On the other hand, the coaxial illumination device 14 illustrated in FIG. 42 is advantageous for miniaturization.

### [Dimming by Diffusion Plate]

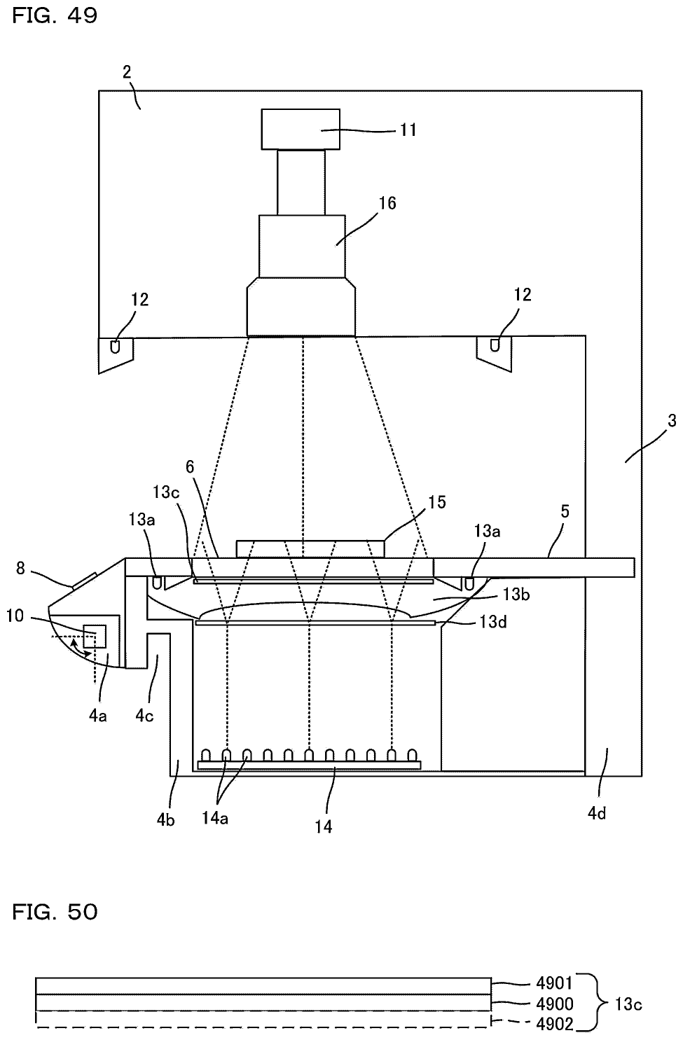

FIG. 49 illustrates a state of test light when the degree of diffusion of the diffusion plate 13c is switched from a first degree of diffusion to a second degree of diffusion. Note that FIG. 42 that has been described above illustrates a state of the test light when the degree of diffusion of the diffusion plate 13c is the first degree of diffusion. In FIG. 42, the test light is diffused by the diffusion plate 13d having the constant degree of diffusion, and further diffused while passing through the diffusion plate 13c to be desired test light. On the other hand, in FIG. 49, the test light is diffused by the diffusion plate 13d having a constant degree of diffusion, but is not diffused or is diffused with a low degree of diffusion to be desired test light when passing through the diffusion plate 13c.

As illustrated in FIG. 42, the test light diffused by the diffusion plate 13c at the first degree of diffusion can reduce the contrast of the test image and reduce the influence of a scratch and dirt on the Petri dish 15. On the other hand, as illustrated in FIG. 49, the test light diffused by the diffusion plate 13c with the second degree of diffusion lower than the first degree of diffusion increases the contrast of the test image, and can increase a count value of colonies. Alternatively, contours of the colonies can be made clear, which is useful to test shapes of the colonies.

When the degree of diffusion is the second degree of diffusion lower than the first degree of diffusion, it is more likely to be affected by the scratch and dirt as compared with the case of the first degree of diffusion. A counting section may count the number of colonies included in the test image based on shape features such as circularity, an aspect ratio, area, and a perimeter. As a result, it is possible to suppress an erroneous count due to the scratch and dirt and to count the number of colonies more accurately.

The user sets the degree of diffusion of the diffusion plate 13c in the dimming control section 13z through the keyboard 32 or the pointing device 33. The dimming control section 13z electrically controls the diffusion plate 13c so as to achieve the set degree of diffusion. For example, the first degree of diffusion may be achieved by applying a first voltage to the diffusion plate 13c, and the second degree of diffusion may be achieved by applying a second voltage to the diffusion plate 13c.

FIG. 50 is a view illustrating a configuration example of the diffusion plate 13c. A diffusion film 4901 is provided on a first surface side of a base material 4900 which is a glass plate or an acrylic plate having translucency. Optionally, a diffusion film 4902 may be provided on a second surface side of the base material 4900. The diffusion films 4901 and 4902 may be liquid crystal films. In this case, the dimming control section 13z turns on/off voltages applied to the diffusion films 4901 and 4902, so that orientations of liquid crystals in the diffusion films 4901 and 4902 are changed, and the degree of diffusion of the diffusion films 4901 and 4902 is changed. For example, when both the diffusion films 4901 and 4902 are off, the second degree of diffusion is achieved. When both the diffusion films 4901 and 4902 are on, the first degree of diffusion is achieved. When the diffusion film 4901 is on and the diffusion film 490 is off, a third degree of diffusion is achieved (second degree of diffusion <third degree of diffusion <first degree of diffusion). When the diffusion film 4901 is off and the diffusion film 490 is on, a fourth degree of diffusion is achieved (second degree of diffusion <fourth degree of diffusion <first degree of diffusion). Here, the third degree of diffusion may be higher or lower than the fourth degree of diffusion. Alternatively, the third degree of diffusion may be equal to the fourth degree of diffusion. As a result, the degree of diffusion of the diffusion plate 13c can be switched in three stages or four stages.

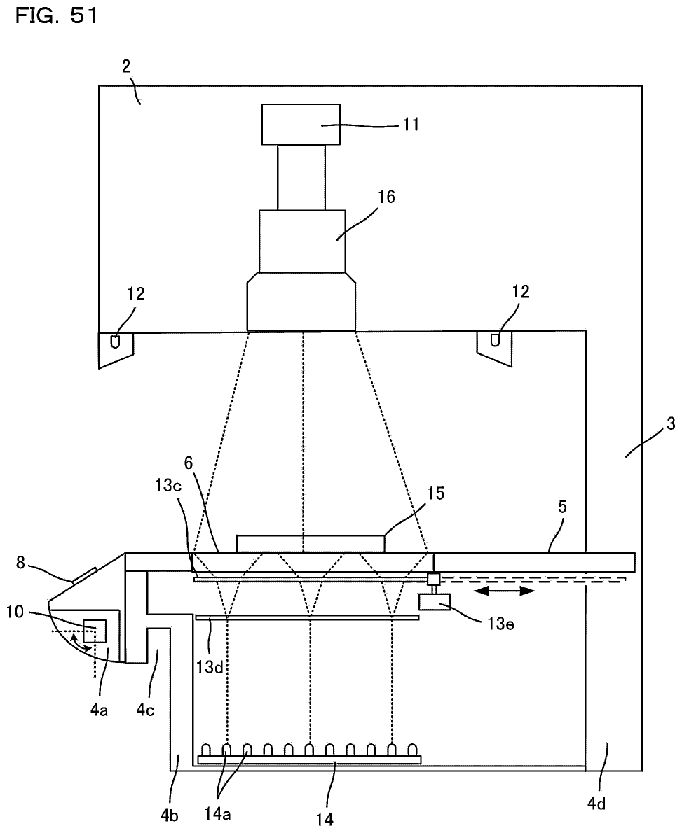

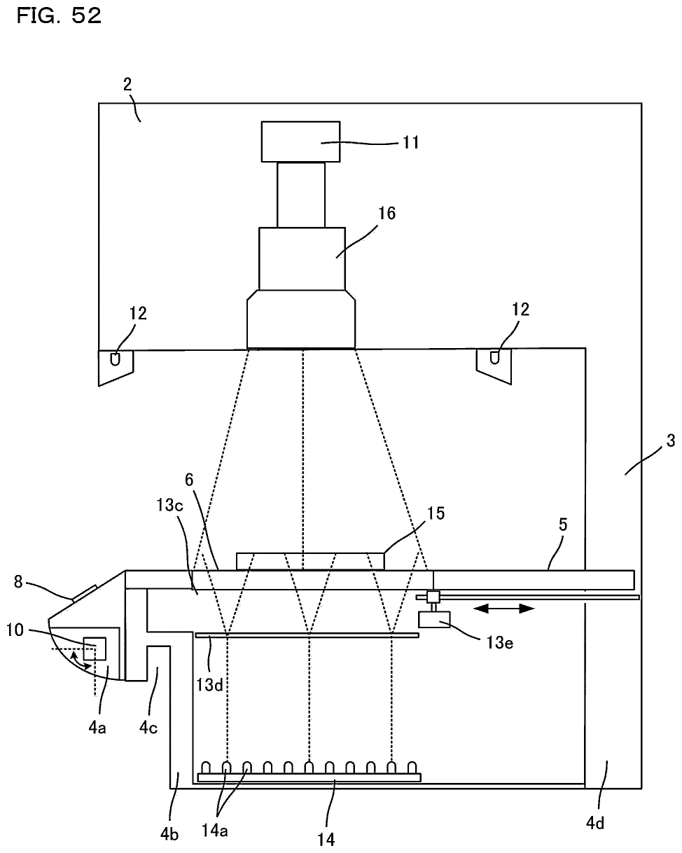

FIGS. 51 and 52 illustrate a method of electrically and mechanically switching the degree of diffusion. The dimming control section 13z can insert the diffusion plate 13c into an optical path of test light (FIG. 51) or extract the diffusion plate 13c from the optical path (FIG. 52) by controlling a motor 13e. The method of electrically and mechanically switching the degree of diffusion can also be implemented in a case where there is a margin in a size of the lower unit 4. Further, in this case, the degree of diffusion of the diffusion plate 13c is constant.

[Setting of Degree of Diffusion and Association with Test Image]

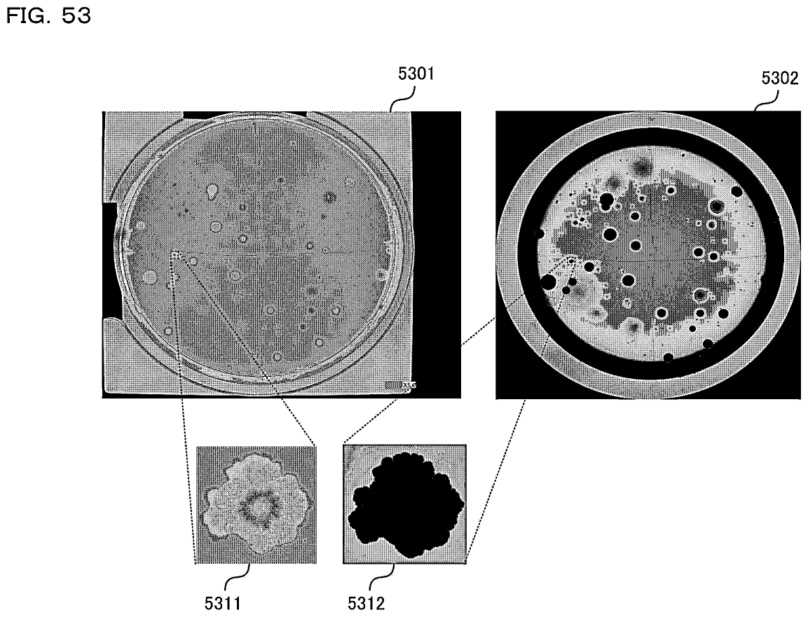

FIG. 53 illustrates a test image 5301 of a test individual irradiated with test light diffused with a high degree of diffusion by the diffusion plate 13c and a test image 5302 of the test individual irradiated with test light diffused with a low degree of diffusion by the diffusion plate 13c. Further, an enlarged image 5311 is an enlarged view of a certain colony included in the test image 5301. An enlarged image 5312 is an enlarged view of the same colony included in the test image 5302.

Since the contrast of the test image 5301 is low, scratches, stains, and the like are not conspicuous in the test image 5301. On the other hand, since the contrast of the test image 5302 is high, the scratches, stains, and the like are conspicuous in the test image 5301. On the other hand, a contour of the colony is not conspicuous in the test image 5301 and the enlarged image 5311 thereof. In the test image 5302 and the enlarged image 5312 thereof, a contour of the colony is conspicuous. That is, a desired test image according to the use can be obtained by switching the degree of diffusion according to the use of the test.

A comparison result of FIG. 53 suggests that an appropriate degree of diffusion varies depending on a bacterial species and a culture medium type. On the other hand, the user can understand a difference in the degree of diffusion for the first time by comparing the test image 5301 and the test image 5302. That is, even if only the test image 5301 or only the test image 5302 is viewed, it is difficult to immediately understand whether the test image is captured with a high degree of diffusion or captured with a low degree of diffusion. In particular, in a case where a user who has acquired the test image is different from a user who observes the test image, or in a case where the test image is observed again after a lapse of several days or several months since the acquisition of the test image, the degree of diffusion may not be immediately recalled from the test image.

Therefore, the degree of diffusion is associated with the cell of the count table and the test image in the present embodiment. As a result, the user can easily know the degree of diffusion associated with the cell or the test image.

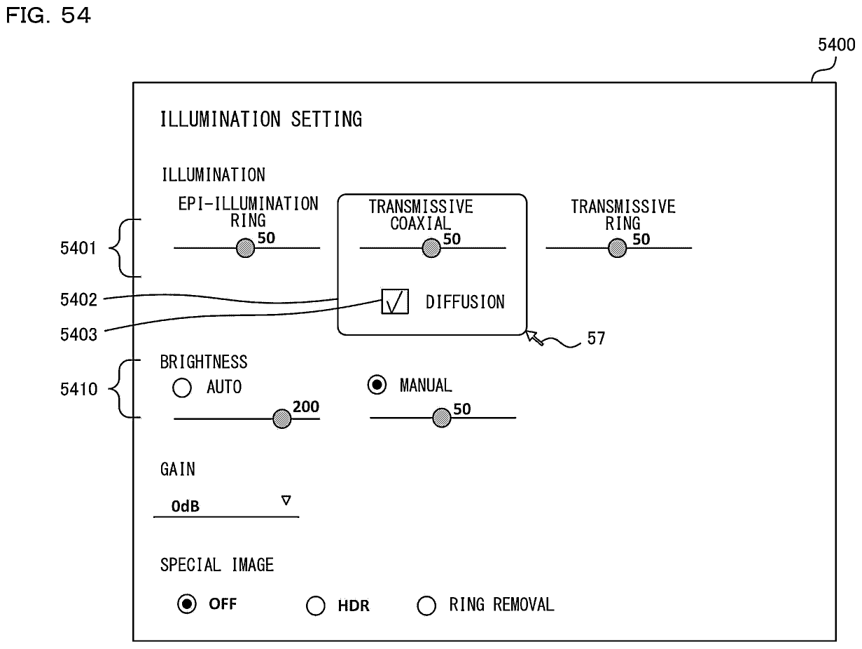

FIG. 54 illustrates an illumination setting UI 5400 that may be called from the illumination button of the dialog 90 illustrated in FIG. 15 or the test condition confirmation screen 110 illustrated in FIG. 17. An illumination selection section 5401 is a UI for selecting an illumination type. In this example, one type can be selected from Epi-illumination ring (the ring illumination device 12), Transmissive coaxial (the coaxial illumination device 14), or Transmissive ring (the ring illumination device 13). A selection frame 5402 is a display object that displays an illumination type currently being selected in an emphasized manner. A check box 5403 is a control object for selecting a degree of diffusion. When the check box 5403 is checked, a high degree of diffusion is selected. When the check box 5403 is unchecked, a low degree of diffusion is selected.

A brightness adjustment section 5410 includes a radio button for selecting a brightness adjustment method (Auto or Manual) and a slide bar for selecting brightness. When Auto is selected, the brightness is automatically determined by the MCU 30 and reflected on the slide bar. When Manual is selected, the brightness is selected according to an operation of the slide bar. In this manner, the brightness determined in this manner is also reflected on the slide bar and a numerical value of the illumination selection section 5401.

Here, the determined illumination type, degree of diffusion, and brightness are associated with a cell that has called the illumination setting UI 5400 among the cells of the count table. As described above, the test image is also associated with each of the cells. For example, a cell of general viable bacteria and a cell of Escherichia coli may be associated with different degrees of diffusion, or may be associated with the same degree of diffusion.

As described above, the test condition, the test image, and the count result are associated with each cell of the count table. Since the degree of diffusion is one of the test conditions, the degree of diffusion is also associated with the test image and the count result.

Although the degree of diffusion is selected using the check box 5403 here, the degree of diffusion may be selected through another control object. For example, a radio button for selecting one degree of diffusion from two or more selectable degrees of diffusion may be adopted. Alternatively, a check box or radio button for turning on/off a function of reducing a scratch and noise (noise reduction function) may be adopted. In this case, the MCU 30 selects a high degree of diffusion when the noise reduction function is turned on, and selects a low degree of diffusion when the noise reduction function is turned off. In this manner, other options on the UI are associated with the degrees of diffusion, and the degree of diffusion may also be indirectly selected by the user selecting one option from a plurality of options.

The culture medium type and the degree of diffusion may be associated in advance. The MCU 30 may receive selection of a type of a culture medium by the user from the culture medium type setting section 96 illustrated in FIG. 15, acquire a degree of diffusion associated with the selected culture medium from the storage device 35, and set the degree of diffusion in the dimming control section 13z.

### [Application of Switching of Degree of Diffusion]

The colony counting device 1 of the present embodiment can acquire a plurality of test images while changing the degree of diffusion. In general, one degree of diffusion with which colonies can be accurately counted is selected, and the colonies are counted from one test image generated with the selected degree of diffusion. However, a plurality of test images to which mutually different types of diffusion are applied may be generated for colony counting.

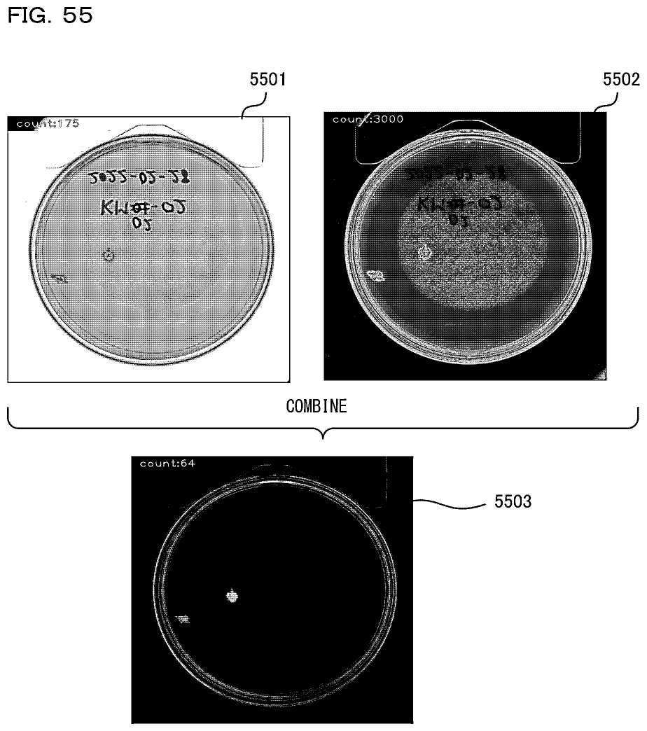

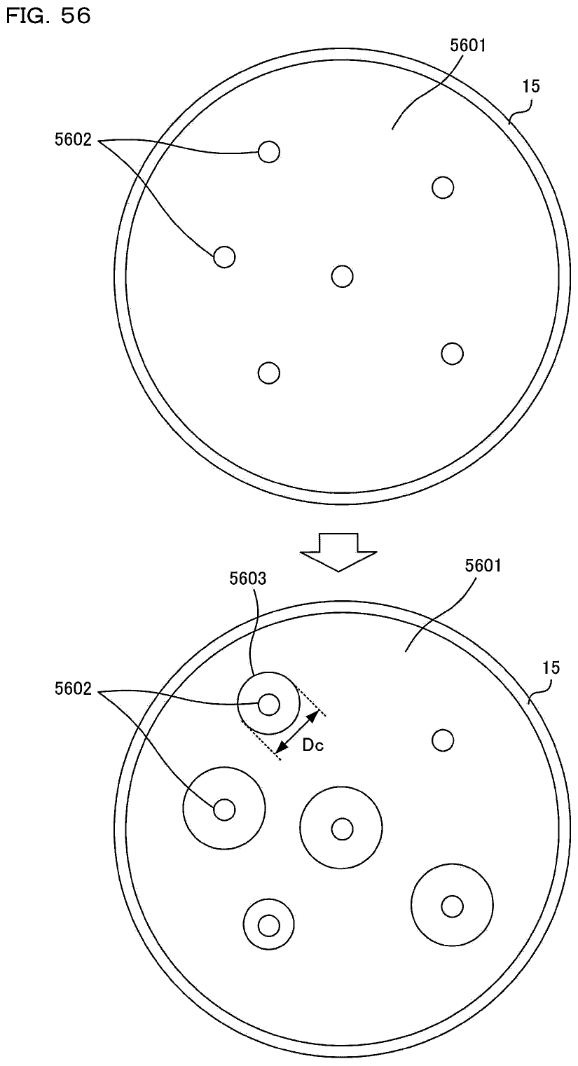

FIG. 55 illustrates that a test image 5501 generated with a high degree of diffusion and a test image 5502 generated with a low degree of diffusion are combined to obtain a combined test image 5503. Here, the ring illumination device 13 of a transmissive type is used as an illumination device. Since the ring illumination device 13 is used, test light passes through only the diffusion plate 13c without passing through the diffusion plate 13d.

When the Petri dish 15 is subjected to culturing, dew condensation sometimes occurs in the Petri dish 15. In particular, in the test image 5502, a contour of the dew condensation is emphasized, and every water droplet of the dew condensation is counted as colonies (for example, count value=3000). On the other hand, in the test image 5501, characters handwritten by the user on the Petri dish are sometimes counted as colonies (for example, count value=175). Therefore, the MCU 30 combines the test image 5501 generated by setting the diffusion plate 13c to the high degree of diffusion and the test image 5502 generated by setting the diffusion plate 13c to the low degree of diffusion to generate the test image 5503, and counts colonies in the test image 5503. As a result, a more accurate number of colonies (for example, 64) is obtained. A combining method may be any method as long as the influence of the dew condensation and the characters is reduced. For example, the MCU 30 may generate the test image 5501 from a difference between the test image 5502 and the test image 5501. As the difference between the test image 5501 and the test image 5502 is calculated in this manner, the combined test image 5503 in which the influence of disturbance observed as a bright area in both the images is suppressed is generated. Note that the combined test image 5503 may be referred to as a disturbance-suppressed image.

### [Measurement of Inhibition Halo]

FIG. 56 is a view illustrating measurement of resistance of bacteria to a drug. It is known that bacteria acquire resistance to a drug. Therefore, it is very useful to examine which drug among a plurality of drugs is effective against a certain bacterium. In FIG. 56, a culture medium 5601 in which bacteria are uniformly applied is accommodated in the Petri dish 15. Six types of drugs 5602 are put or dropped thereto.

When the drug 5602 is effective against bacteria, a plurality of fungi existing around the drug 5602 are killed. A circular area of the culture medium 5601 where the fungi have been killed is called an inhibition halo 5603. A diameter Dc of the inhibition halo 5603 indicates the efficacy of the drug against bacteria. As the diameter Dc of the inhibition halo of a certain drug is smaller, bacteria have acquired stronger resistance to the drug. As the diameter Dc of the inhibition halo of a certain drug is larger, the drug has stronger efficacy against bacteria.

An effective degree of diffusion for measuring the diameter Dc of the inhibition halo may vary depending on a culture medium type. Therefore, the MCU 30 receives an input of a culture medium type by the user, and selects a degree of diffusion according to the culture medium type among the plurality of degrees of diffusion to generate a test image for measuring the diameter Dc of the inhibition halo. The storage device 35 may store the culture medium types and the degrees of diffusion in association with each other in advance. For example, the MCU 30 may display a UI for associating the culture medium types with the degrees of diffusion on a one-to-one basis on the display device 37, and receive a setting for associating the degrees of diffusion with the culture medium types through the UI. The MCU 30 refers to the storage device 35 to identify a degree of diffusion corresponding to a culture medium type input when the inhibition halo is measured, and sets the identified degree of diffusion in the dimming control section 13z. The dimming control section 13z applies a voltage to the diffusion plate 13c so as to achieve the set degree of diffusion. As a result, the degree of diffusion suitable for the culture medium is adopted, and thus, the MCU 30 can accurately measure the diameter Dc of the inhibition halo 5603 from the test image.

### [Facilitation of Adjustment of Test Parameters]

### (1) Basic Concept

Adjustment of test parameters have been one of difficult tasks for the user. A counting result of colonies obtained by the colony counting device 1 is required to substantially coincide with a counting result obtained by a human. For this purpose, an imaging condition, an illumination condition, an image processing condition (counting condition), and the like need to be appropriately adjusted. In general, a test individual illuminated according to the illumination condition is captured by the main camera 11 according to the imaging condition, thereby generating an image of the test individual. The image of the test individual is subjected to shading correction, brightness conversion, noise reduction processing (for example, particle reduction or lint reduction), watershed (a type of image division algorithm), appropriate combination of area sets, and the like according to the image processing condition, and finally converted into a binarized image based on a binarization sensitivity (binarization threshold). Therefore, binarization conversion processing is extremely complicated arithmetic processing and requires a considerable operation time. Thereafter, the MCU 30 counts the number of colonies from the binarized image. The user needs to compare an original image and the binarized image or confirm a counting result of the colonies to determine which test parameter is to be fine-tuned in which manner. As such a series of adjustment work is repeated for each test parameter, final test parameters are decided. In particular, since the binarized image processing takes time, the user has been kept waiting for a long time from the adjustment of the test parameters to acquisition of the counting result which is a result of the adjustment.

Therefore, the present embodiment facilitates the work of adjusting the test parameters. Further, a part of the present embodiment will shorten the time required for the adjustment work as compared with the related art.

Specifically, the MCU 30 prepares a plurality of candidates of a certain test parameter in advance, and applies the plurality of candidates to an image of a test individual to obtain a binarized image and a counting result in advance. Moreover, the MCU 30 displays a plurality of binarized images and counting results obtained by applying the plurality of candidates, respectively, on the display device 37. The user selects one detection result from a plurality of detection results (binarized images and counting results) displayed on the display device 37 using the pointer 57. The MCU 30 decides the candidate of the test parameter used to obtain the selected detection result as an official test parameter (adjusted test parameter). Since the user can adjust the test parameter only by selecting the detection result that suits his/her sense, the adjustment work will be extremely easy.

Note that the test parameter candidates may be gradually narrowed down such as primary candidates, secondary candidates, . . . , and a final candidate. For example, a plurality of test parameters as the nth-order candidates include one test parameter selected by the user from a plurality of test parameters included in the (n−1)th-order candidates and several test parameters located before and after the one test parameter. In this manner, the test parameter close to the user's sense may be gradually selected.

### (2) User Interface

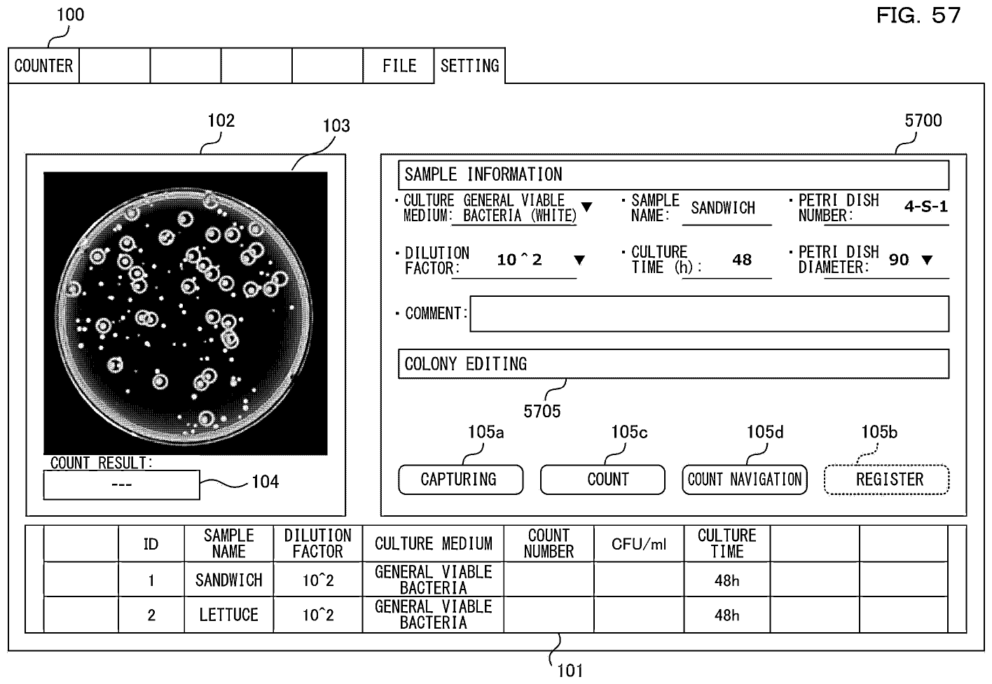

FIG. 57 illustrates the UI 100 (a test result confirmation screen) before the test parameters are adjusted. The description of display objects already described in the UI 100 will be omitted. An information display area 5700 is an area for displaying information on the test individual (sample). The colony editing section 5705 is an area for displaying the adjusted test parameters and the like. Note that the capture button 105a is a button for instructing the MCU 30 to re-capture the test individual. A count button 105c is a button that is pressed for instructing the MCU 30 to count colonies. A count navigation button 105d is a button for instructing the MCU 30 to display a UI for discretely adjusting the test parameters. The registration button 105b is a button for instructing the MCU 30 to register a count result in the count table.

In the result area 102, a moving image captured in real time by the main camera 11 is displayed. However, in the result area 102, the test image 103 as a still image acquired by the main camera 11 based on the illumination condition and the imaging condition set at that time may be displayed. For example, when the count button 105c is pressed while the moving image is displayed in the result area 102, the MCU 30 acquires the test image 103 as the still image and executes a count operation. In FIG. 57, the count result is not yet displayed in the count value area 104, but the count result is displayed in the count value area 104 when the count result is obtained from the test image 103 as the still image.

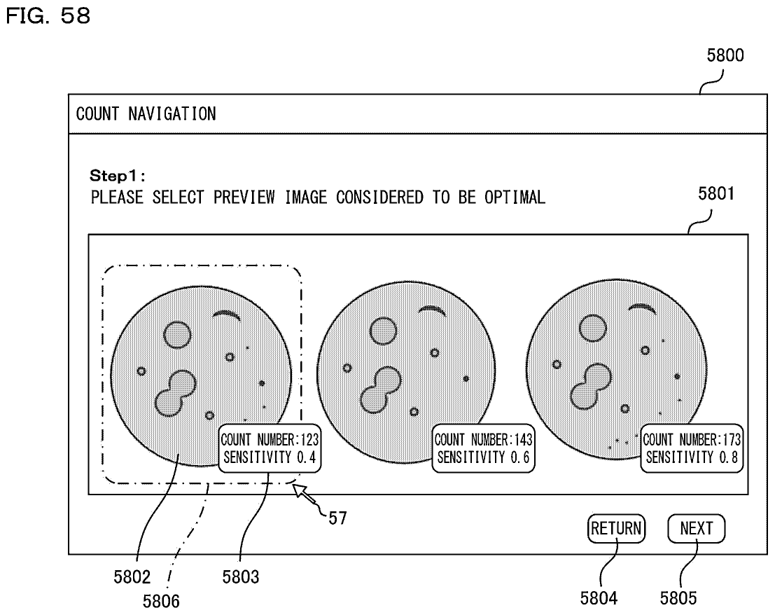

FIG. 58 illustrates a count navigation UI 5800 displayed on the display device 37 when the count navigation button 105d is pressed. The count navigation UI 5800 includes a candidate display area 5801. The candidate display area 5801 is an area for displaying a candidate image, a candidate parameter, and a count number. In this example, a plurality of binarized images (candidate images 5802) respectively generated by applying a plurality of test parameter candidates (candidate parameters) of the same type are displayed. An information display area 5803 is an area for displaying a test parameter (for example, sensitivity (abbreviation of the binarization sensitivity)) applied to obtain the candidate image 5802 and a count number. The test parameter may be referred to as a colony detection parameter.

In this example, three candidate images 5802 obtained by mutually applying different test parameters are displayed. The user selects one candidate image 5802 by operating the pointer 57. A selection frame 5806 is a box or a frame for displaying the candidate image 5802 selected by the user in an emphasized manner.

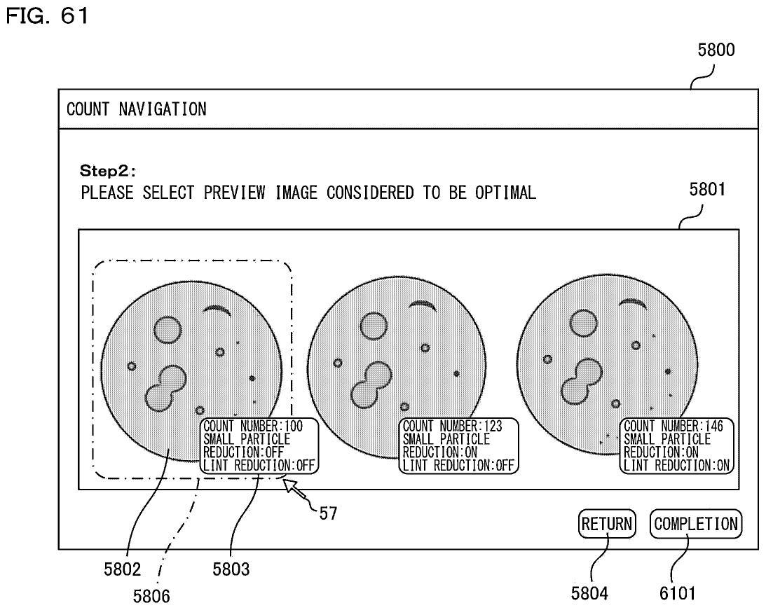

When detecting that a return button 5804 is pressed by the pointer 57, the MCU 30 returns from the count navigation UI 5800 to the UI 100. When detecting that a next button 5805 is pressed, the MCU 30 transitions to the next UI (for example, the UI 5800 illustrated in FIG. 61).

Meanwhile, although the three candidate images 5802 are displayed in FIG. 58, this is merely an example. The number of candidate images 5802 displayed in the candidate display area 5801 may be two or more.

When a part of the candidate image 5802 is clicked by the pointer 57, the MCU 30 may enlarge and display the part (a zoom function). Moreover, the MCU 30 may translate an enlargement position when a part of the candidate image 5802 is dragged by the pointer 57. At that time, the MCU 30 may execute zooming or panning simultaneously and in parallel on all of the three candidate images 5802. As a result, it is possible to simultaneously compare details of the three candidate images 5802.

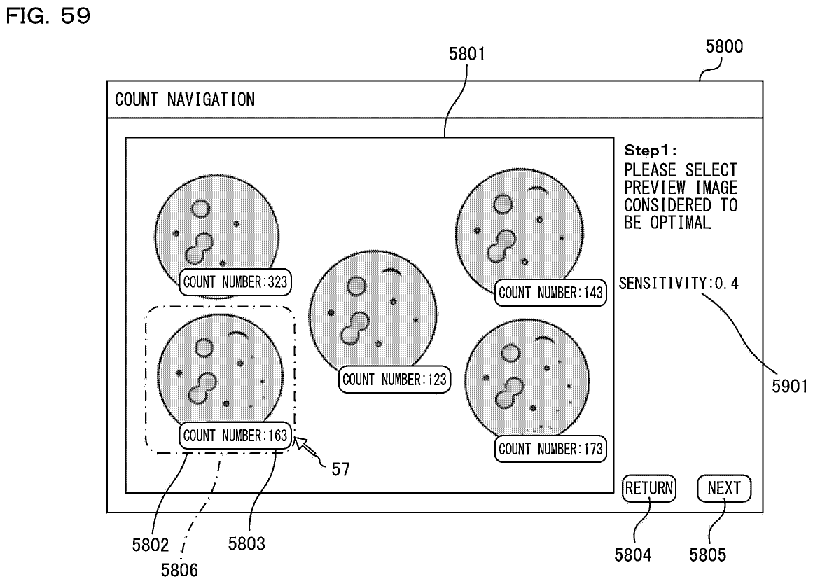

FIG. 59 illustrates another example of the count navigation UI 5800. In this example, five candidate display images 5802 are displayed in the candidate display area 5801. However, as the number of candidate images 5802 displayed in the candidate display area 5801 increases, the visibility of the candidate image 5802 decreases, or the amount of information that can be displayed in the information display area 5803 decreases. In this example, the information display area 5803 can display only the count number, and a display area 5901 of the test parameter (sensitivity) is provided at another position of the count navigation UI 5800. The display area 5901 displays the test parameter used to obtain the candidate image 5802 selected by the pointer 57.

Meanwhile, FIGS. 58 and 59 suggest another aspect of the present embodiment. The MCU 30 can calculate detection results (for example, the candidate images 5802 and the count numbers) for N test parameters of the same type (for example, sensitivity: 0.2, 0.4, 0.6, 0.8, and 1.0) in advance. For example, as illustrated in FIG. 59, five detection results may be calculated in advance. Moreover, the MCU 30 may actually display only M detection results among the N detection results in the candidate display area 5801 (N and M are integers, and N>M). For example, as illustrated in FIG. 58, only three detection results may be displayed. As the calculation is executed in advance in this manner, the waiting time of the user at the time of switching the candidate image 5802 will decrease.

Moreover, FIGS. 58 and 59 suggest still another aspect of the present embodiment. For example, it may be understood that FIG. 59 illustrates primary candidates and FIG. 58 illustrates secondary candidates. For example, 0.4 may be selected as the sensitivity from the primary candidates (for example, sensitivity: 0.2, 0.4, 0.6, 0.8, and 1.0), and 0.3, 0.4, and 0.5 may be displayed to be selectable as the sensitivities from the secondary candidates. Note that selecting the candidate image 5802 corresponds to indirectly selecting the test parameter associated with the candidate image 5802. Moreover, the MCU 30 may determine tertiary candidates from test parameters selected from the secondary candidates, and cause the user to select one test parameter from the tertiary candidates. In this manner, with the progress from the primary candidates to the nth-order candidates, the test parameters can be gradually narrowed down to a test parameter considered to be appropriate by the user (n is an integer of three or more). Note that an interval (difference) between the test parameter candidates may be gradually reduced with the progress from the primary candidates to the nth-order candidates.

Further, when two candidates (for example, 0.2 and 0.4) are selected as the (n−1)th order candidates, the nth order candidates may be determined to include three or more candidates (for example, 0.25, 0.3, and 0.35) centered on an intermediate value (for example, 0.3) of the two candidates.

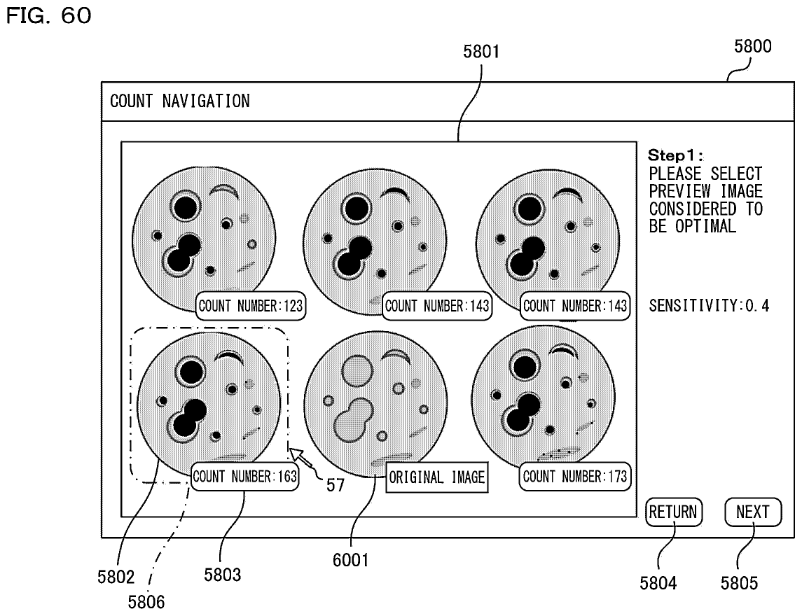

FIG. 60 illustrates still another example of the count navigation UI 5800. In this example, an original image 6001 is a raw image (color image or grayscale image) acquired from a test individual. That is, the original image 6001 is an image close to an impression of the user viewing the Petri dish 15 with the naked eye. On the other hand, the candidate image 5802 is an image generated by superimposing a binarized image, generated by applying a test parameter to the original image 6001 and performing image processing, on the original image 6001. In this case, the user can determine whether or not colonies present in the original image 6001 are correctly detected by confirming the binarized image superimposed on the original image 6001. That is, the user will be able to know whether or not image areas recognized by the user as colonies are also detected as colonies by the MCU 30. In this case, the binarized image superimposed on the original image 6001 will function as markers indicating detection positions of the colonies. Note that the MCU 30 may display markers (for example, red cross marks or the like) superimposed on image areas actually detected as the colonies.

FIG. 61 illustrates the count navigation UI 5800 displayed when the next button 5805 is pressed. Here, it is assumed that the sensitivity is adjusted in Step 1, and the other test parameters are adjusted in Step 2. This may be referred to as two-step adjustment. Note that while (or before) the user selects the sensitivity in Step 1, the MCU 30 may calculate in advance detection results (candidate images and count numbers) to which combinations of the other test parameters are applied for sensitivity candidates, respectively, and store the detection results in the storage device 35. When the next button 5805 is pressed, the MCU 30 may read the detection results of Step 2 from the storage device 35 and display a plurality of detection results (candidates) in the candidate display area 5801.

Note that it is assumed that a calculation load of the test parameter adjusted in Step 1 is larger than a calculation load of the test parameters adjusted in Step 2. Examples of the test parameter adjusted in Step 1 include the intensity of noise removal, the sensitivity (binarization threshold), and on/off of shading correction. Examples of the test parameters adjusted in Step 2 include on/off of a small particle reduction function, a size of shape division, on/off of a lint reduction function, on/off of a large particle reduction function, and on/off of an expansion/contraction function.

In this example, on/off of the small particle reduction function and on/off of the lint reduction function are adopted as the other test parameters. That is, the three candidate images 5802 are candidate images generated by applying the common sensitivity and applying combinations (three combinations among four combinations) of on/off of the small particle reduction function and on/off of the lint reduction function. The information display area 5803 indicates the count number and the test parameters of a second type (on/off of the small particle reduction function and on/off of the lint reduction function). When detecting that the user selects one candidate image 5802 and presses a completion button 6101, the MCU 30 stores a series of test parameters applied to generate the selected candidate image 5802 in the storage device 35 as official test parameters.

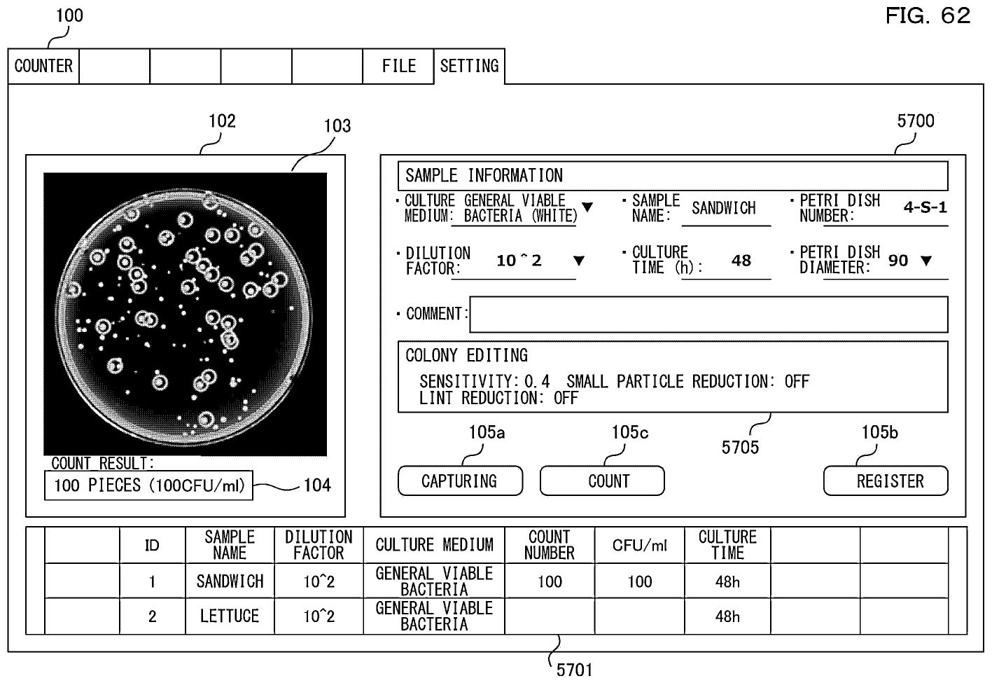

FIG. 62 illustrates a result confirmation screen (UI 100) displayed when the completion button 6101 is pressed. The colony editing section 5705 displays the test parameters adjusted through the count navigation UI 5800. The MCU 30 may display the adjusted test parameters in red, bold, a blinking manner, or the like to achieve emphasized display.

The count value area 104 displays a count number to which the plurality of types of test parameters decided through the UI 5800 are applied. The count number is further registered in the count table and displayed on the UI 100.

Although the count navigation button 105d is not provided in FIG. 62, the count navigation button 105d may be provided. In this case, once the plurality of types of test parameters are adjusted, the user can re-adjust the plurality of types of test parameters by pressing the count navigation button 105d. Note that a test parameter decided in the previous adjustment may be read from the storage device 35 and adopted as a median of a plurality of test parameters to be primary candidates in the re-adjustment.

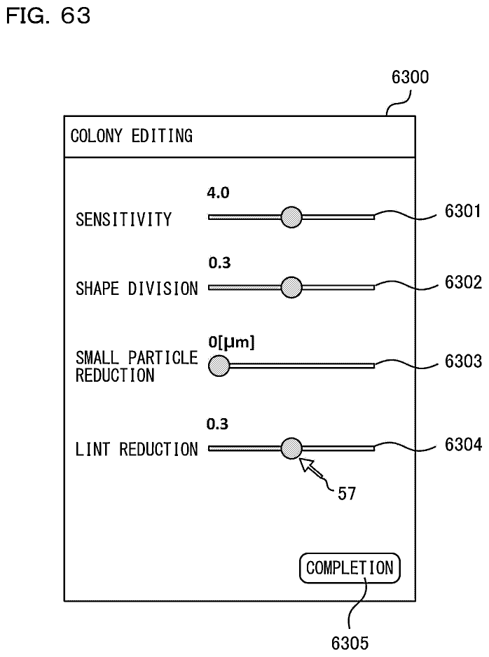

FIG. 63 illustrates a UI 6300 displayed by double-clicking or right-clicking the colony editing section 5705 with the pointer 57. The UI 6300 may have control objects that can consecutively adjust the test parameters. A slide bar 6301 is a slide bar for further fine adjustment of the sensitivity that has been adjusted through the UI 5800. A slide bar 6302 is a slide bar for adjusting a size when a shape detected as a colony is to be divided. A slide bar 6303 is a slide bar for adjusting a reduction effect when the small particle reduction function is set to on. A slide bar 6304 is a slide bar for adjusting a reduction effect when the lint reduction function is set to on. When detecting that a completion button 6305 is pressed, the MCU 30 decides four test parameters that have been adjusted by the slide bars 6301 to 6304 at that time, stores the decided parameters in the storage device 35, and transitions from the UI 6300 to the UI 100.

### (3) Flowchart

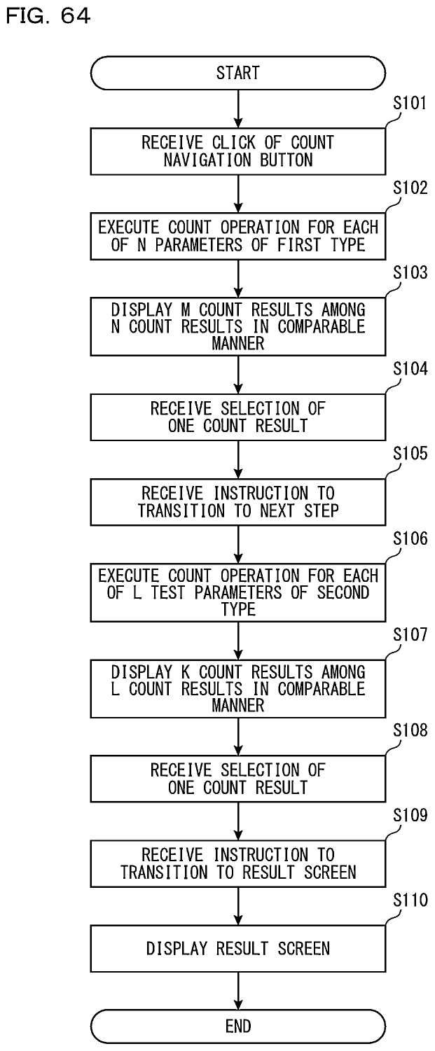

FIG. 64 is a flowchart illustrating a test parameter adjustment method executed by the MCU 30. Here, it is assumed that the UI 100 is displayed on the display device 37.

In S101, the MCU 30 (an instruction receiving section) receives a click of the count navigation button 105d by the pointing device 33 in the UI 100.

In S102, the MCU 30 (an image processing section, a calculation section, or a counting section) executes a count operation for each of N test parameters of a first type (for example, sensitivity). For example, five sensitivity values, different from each other, are applied to an image of a test individual, and the count operation is executed. Here, the count operation refers to binarizing the image of the test individual and counting the number of colonies from the generated binarized image. In the count operation, default values stored in the storage device 35 are adopted as test parameters of another type. N count results (detection results) are stored in the storage device 35.

In S103, the MCU 30 (a display processing section) displays M count results among the N count results in a comparable manner in the UI 5800. Basically, N>M, but N=M may be satisfied.

In S104, the MCU 30 (a selection section) receives selection of one count result from the M count results. As described above, one candidate image 5802 may be selected by the pointer 57.

In S105, the MCU 30 (the instruction receiving section) receives an instruction to transition to the next step. This transition instruction is, for example, a press of the next button 5805.

In S106, the MCU 30 (the image processing section, the calculation section, or the counting section) executes a count operation for each of L test parameters of a second type. As a result, L count results are obtained and stored in the storage device 35. Examples of the second type test parameters include on/off of the small particle reduction function, on/off of the lint reduction function, and a size of shape division.

In S107, the MCU 30 (the display processing section) displays K count results among the L count results in a comparable manner in the count navigation UI 5800. Basically, L>K, but L=K may be satisfied for the reason of N and M and shaking described above. Note that a case where L=4 and M=3 has been introduced in FIG. 61.

In S108, the MCU 30 (the selection section) receives selection of one count result among the K count results. As described above, one candidate image 5802 is selected by the pointer 57.

In S109, the MCU 30 (the instruction receiving section) receives an instruction to transition to a result screen (for example, the UI 100) by the pointing device 33. This transition instruction is, for example, a press of the completion button 6101.

In S119, the MCU 30 (the display processing section) displays the result screen (for example, the UI 100) on the display device 37.

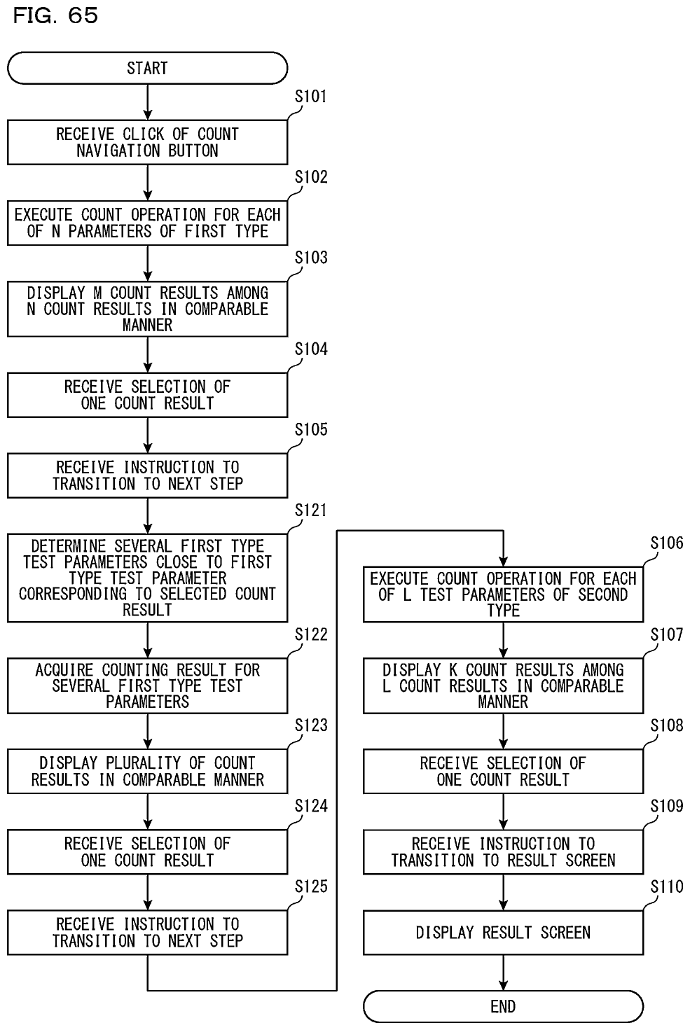

FIG. 65 is a flowchart illustrating a test parameter adjustment method executed by the MCU 30. Here, it is assumed that the UI 100 is displayed on the display device 37. As compared with FIG. 64, S121 to S125 are inserted between S105 and S106 in FIG. 65.

In S121, the MCU 30 (a determining section) determines several first type test parameters close to the first type test parameter corresponding to the selected count result in order to acquire secondary candidates. For example, it is assumed that there are 0.2, 0.4, 0.6, 0.8, and 1.0 as primary candidates of the sensitivity, and 0.4 is selected therefrom. In this case, the secondary candidates of the sensitivity are 0.3, 0.4, and 0.5.

In S122, the MCU 30 (an acquisition section) acquires count results for the determined several first type test parameters. For example, when the secondary candidates of the sensitivity are 0.3, 0.4, and 0.5, a count result with the sensitivity of 0.3, a count result with the sensitivity of 0.4, and a count result with the sensitivity of 0.5, which are stored in the storage device 35 in advance, are read. Alternatively, the MCU 30 performs a count operation to obtain each of the count result with the sensitivity of 0.3, the count result with the sensitivity of 0.4, and the count result with the sensitivity of 0.5.

In S123, the MCU 30 (the display processing section) displays the plurality of count results in a comparable manner.

In S124, the MCU 30 (the selection section) receives selection of one count result from the plurality of count results displayed in a comparable manner.

In S125, the MCU 30 (the instruction receiving section) receives an instruction to transition to the next step. This transition instruction is, for example, a press of the next button 5805.

In this manner, the first type test parameters such as the sensitivity may be narrowed down stepwise. As a result, the user can more precisely adjust the first type test parameter. Note that such stepwise narrowing processing may also be applied to the second type test parameters.

The decided test parameter may be stored in the storage device 35 in association with a sample name and a culture medium type. When the UI 100 is called next time, the MCU 30 reads the previous test parameter stored in the storage device 35 to be used as a test parameter serving as the center (median) for creating the candidate image 5802. In this case, the MCU 30 may calculate the other candidate test parameters from the previous test parameter to generate a test parameter group as primary candidates. The plurality of test parameters forming the test parameter group and serving as the primary candidate can be calculated as discrete numerical values at regular intervals. Here, the interval (parameter interval) may be determined according to the culture medium type. For example, the storage device 35 may store a pair of the culture medium type and the interval in a table or a database.

### [Modified Example of UI]

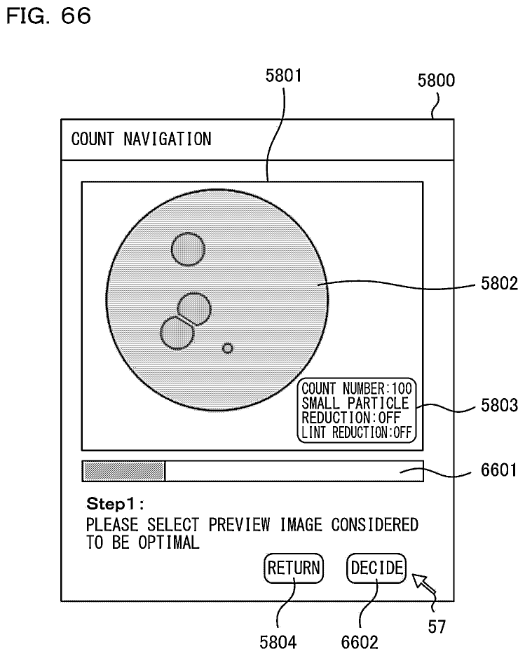

FIGS. 66, 67, and 68 illustrate another example of the count navigation UI 5800 displayed on the display device 37 when the count navigation button 105d is pressed. The same reference numerals are given to parts that have been described in relation to FIG. 58 and the like, and the description thereof will be omitted.

In the UI 5800 illustrated in FIG. 58 and the like, the plurality of binarized images (candidate images 5802) respectively generated by applying the plurality of test parameter candidates (candidate parameters) of the same type are displayed. However, the UI 5800 illustrated in FIG. 66 displays only one binarized image (candidate image 5802) among the plurality of generated binarized images. Therefore, the area of display per candidate image 5802 increases, and the user is more likely to visually confirm details of the candidate image 5802.

The UI 5800 illustrated in FIG. 66 may include a slide bar 6601 as a control object for instructing to switch the candidate image 5802 displayed in the candidate display area 5801. The user switches the candidate image 5802 displayed in the candidate display area 5801 by clicking or dragging the slide bar 6601 with the pointer 57. Here, a first candidate image generated by applying a first test parameter is switched to a second candidate image generated by applying a second test parameter. A test parameter, applied when an image to be displayed in the candidate display area 5801 is generated, is discretely switched from the first test parameter to the second test parameter, and the candidate images before and after the test parameter is discretely switched are sequentially displayed at the same position in the candidate display area 5801, so that the user can easily grasp a difference between the candidate image in the case of applying the first test parameter and the candidate image in the case of applying the second test parameter and a difference between counting results. That is, in this case, the user can recognize the difference between the candidate image in the case of applying the first test parameter and the candidate image in the case of applying the second test parameter and the difference between the counting results without moving the line of sight on a screen or visually extracting a difference portion.

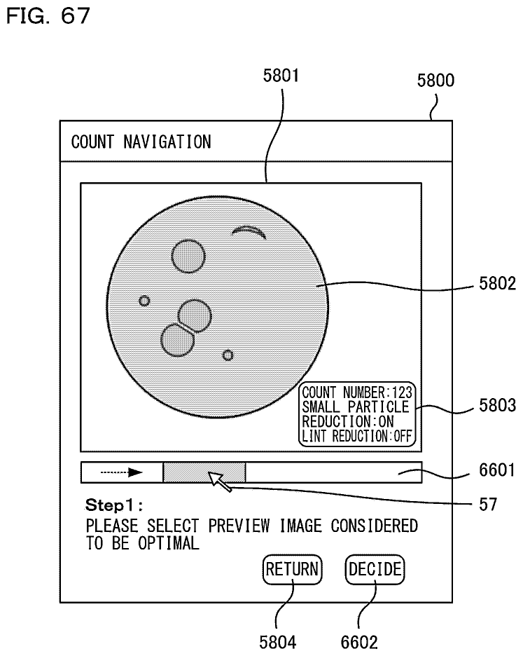

FIG. 67 illustrates that the candidate image 5802 has been switched by the user. In this example, the slide bar 6601 is dragged in the right direction, and another (second) candidate image 5802 is displayed in the candidate display area 5801. A test parameter and a count number displayed in the information display area 5803 are also switched according to the candidate image 5802.

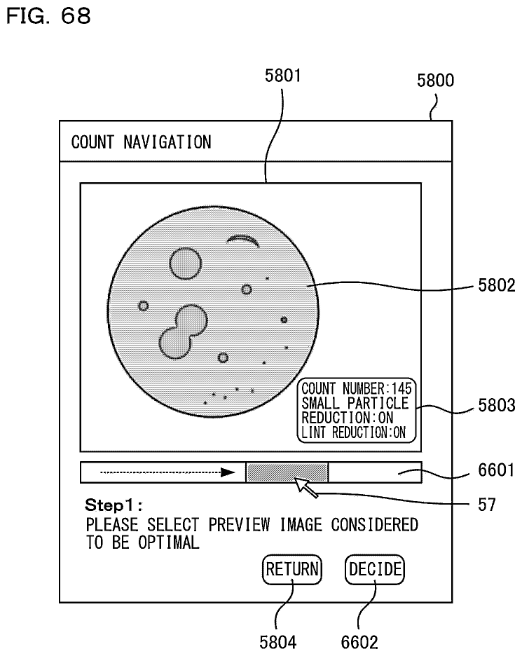

FIG. 68 illustrates that the candidate image 5802 has been switched by the user. In this example, the slide bar 6601 is further dragged in the right direction, and still another (third) candidate image 5802 is displayed in the candidate display area 5801. A test parameter and a count number displayed in the information display area 5803 are also switched according to the candidate image 5802.

When a decide button 6602 provided in the UI 5800 is selected and pressed by the pointer 57, the candidate image 5802 displayed in the candidate display area 5801 and the test parameters displayed in the information display area 5803 are decided as those selected by the user at that time. The plurality of candidate images 5802 may be displayed in a comparable manner by switching the plurality of candidate images 5802 using the slide bar 6601 as described above. In particular, the candidate images 5802, obtained by discretely changing the test parameters, can be displayed at the same position in the candidate display area 5801 while being sequentially switched by sequentially dragging the slide bar 6601 in the right direction or the left direction. As a result, it is easy to grasp which area of the candidate image 5802 is newly counted as a colony and how the candidate image 5802 has changed by changing the test parameters.

The slide bar 6601 may be operated by a cursor key provided on a keyboard.

Note that the candidate image 5802 with a smaller count number may be displayed as the slide bar 6602 advances to the left, and the candidate image 5802 with a larger count number may be displayed as the slide bar 6602 advances to the right. In this manner, (the display order of) the plurality of candidate images 5802 may be sorted depending on the count numbers.

### [Others]

In the analysis industry, a laboratory information management system (LIMS) has attracted attention. The LIMS is a type of application, and forms a database of a test individual and a test condition, a test image, a test result, a test flow, a test device, and the like related thereto. As a result, it is unnecessary to organize documents in a laboratory and it possible to manage test data efficiently and with transparency. The colony counting device 1 cooperates with a count table or associates the test condition with the test result, thereby playing a part of the LIMS. In particular, since a degree of diffusion is managed in association with the test image, it is possible to verify the test condition (particularly, the degree of diffusion) of a test at a later date.

[Technical Ideas Derived from Embodiment]

### [Viewpoint A1]

The storage device 35 is an example of a storage section that stores a count table including a cell to which a count result of each of a plurality of test individuals is input. The MCU 30 and the display control section 36 are an example of a display control section that displays the count table stored in the storage section on the display device 37. The MCU 30, the pointing device 33, and the like are examples of a cell identifying section that identifies a target cell to which a count result is input from among a plurality of the cells included in the count table displayed by the display control section. The MCU 20 or the MCU 30 is an example of a counting instruction section that generates a counting instruction according to an operation of a user. The MCU 30 and the main camera 11 are an example of an acquisition section that acquires a test image that is an image of the test individual based on the counting instruction generated by the counting instruction section. The MCU 20 or the MCU 30 is an example of a counting section that counts colonies included in the test individual based on the test image acquired by the acquisition section. As illustrated in FIGS. 19 and 20, the MCU 20 or the MCU 30 functions as a table management section that reflects the number of the colonies counted by the counting section in the target cell. As a result, burden on the user regarding post-processing on colony counting results is mitigated.

### [Viewpoint A2]

The table management section (for example, the MCU 30) may create a count table including an identification information cell, which stores identification information of the test individual, and a count result cell, which is associated with the identification information cell and stores a count result of the number of the colonies for the test individual, and stores the count table in the storage section according to an operation of the user. That is, as illustrated in FIGS. 5 and 16, the count tables 55 and 82 may include the identification information cell that stores the identification information (for example, a sample name) of the test individual and the count result cell that is associated with the identification information cell and stores the count result of the number of the colonies for the test individual. As a result, the user can save time and effort for creating the count table by handwriting.

### [Viewpoint A3]

The table management section (for example, the MCU 30) may associate a test condition with the count result cell that stores the count result of the number of the colonies. As a result, it is possible to easily set the test condition when executing a test related to a colony by associating the test condition with the cell in which the count result is stored.

### [Viewpoint A4]

The acquisition section may include an illumination section (for example, the ring illumination devices 12 and 13 or the coaxial illumination device 14) that illuminates the test individual and an imaging section (for example, the main camera 11) that captures an image of the test individual illuminated by the illumination section. The test condition may include an illumination condition (for example, an illumination type or brightness) of the illumination section. An appropriate illumination condition varies depending on a bacterial species such as Escherichia coli or general viable bacteria, and a type of culture medium (for example, a sheet type medium, a liquid type medium, or a selective medium). Therefore, since the illumination condition is included as the test condition, the illumination condition suitable for each cell can be set. Moreover, the test condition may include an imaging condition (for example, exposure time) of the imaging section. An appropriate imaging condition may vary depending on a culture medium color and a colony color. Since the test condition includes the imaging condition, an appropriate imaging condition can be set for each cell.

### [Viewpoint A5]

The illumination section may operate according to either a first illumination mode (for example, a mode of turning on the ring illumination device 12) in which epi-illumination is performed on the test individual or a second illumination mode (for example, a mode of turning on the coaxial illumination device 14) in which transmitted illumination is performed on the test individual from a direction opposing the imaging section. The test condition includes selection of the first illumination mode or the second illumination mode. When the test condition includes designation of an illumination mode, it is possible to select an appropriate illumination mode for each cell.

### [Viewpoint A6]

The test condition may include a counting condition to be applied to the counting section. Here, the counting condition may include at least one of a threshold for detecting a colony and a color serving as a reference in detecting the colony. For example, the counting condition may include a threshold for distinguishing a colony from the others (for example, a binarization threshold that affects detection sensitivity) and a color that serves as a reference in counting the colony (for example, a foreground color or a background color). The MCU 20 or the MCU 30 may binarize the test image to count the number of colonies. Thus, the binarization threshold affects the detection sensitivity of the colony. When the binarization threshold is appropriately set, erroneous detection of the colony decreases. Further, if the color of the colony and a color of a culture medium can be appropriately set, the erroneous detection of the colony decreases. When the counting condition is set for each cell, the erroneous detection of the colony may decrease for each cell. Further, a counting algorithm may be appropriately adjustable according to the counting condition.

### [Viewpoint A7]

When the counting instruction is input by the user, the counting section (for example, the MCU 20 or the MCU 30) may output an illumination command according to the test condition associated with the target cell to the illumination section. The illumination section illuminates the test individual according to the illumination command. The imaging section captures the image of the test individual illuminated by the illumination section according to the illumination command and generates the test image. The counting section counts the number of colonies based on the test image reflecting the illumination command. As a result, it is possible to count the number of colonies for the test image reflecting the test condition set for each cell.

### [Viewpoint A8]

When the identifying section changes the target cell from a first cell to a second cell, the counting section (for example, the MCU 20 or the MCU 30) changes a test condition to be applied to the acquisition section from a first test condition associated with the first cell to a second test condition associated with the second cell. In this manner, when the target cell is changed, the test condition can be changed in conjunction with the change. An appropriate test condition may vary for each cell, that is, for each test individual. If an appropriate test condition is set for each cell in advance, the user can select an appropriate test condition only by selecting a cell.

### [Viewpoint A9]

The sample DB 40 is a database for assisting creation of the count tables 55 and 82. The MCU 30 may function as a registration section that registers data in the database. The count tables 55 and 82 may have a plurality of row elements each including the identification information cell and the count result cell. The registration section (MCU 30) may be configured to register a row element included in the count table 82 in which the count result has been input to the count result cell in the database. Here, the row element includes a cell and a test condition associated with the cell. The table management section (MCU 30) may create a new count table based on a row element designated by the user among the plurality of row elements held in the database. For example, a cell constituting the row element designated by the user and a test condition associated with the cell are copied to the new count table. Further, the count result that has been stored in the cell may be deleted when being registered in the sample DB 40. Since row elements adoptable as row elements of a count table are stored as the database in advance in this manner, the user can easily create the new count table.

### [Viewpoint A10]

The count tables 55 and 82 may have a plurality of column elements. The plurality of column elements may be associated with each combination. Here, the combination is a combination of a culture condition (for example, a dilution factor or a culture time) of the test individual and a bacterial species (for example, a general viable bacteria or a Escherichia coli). Each of the column elements has a different combination of the culture condition and the bacterial species. For example, a first column element and the second column element are different in at least one of the culture condition and the bacterial species. As a result, for a certain test individual, cells corresponding to a plurality of combinations formed with various culture conditions and various bacterial species can also be grouped into one row.

### [Viewpoint A11]

The table management section (for example, the MCU 30) may determine whether or not a row element (designated row element) designated by the user among the plurality of row elements held in the database is included in a new count table. When it is determined that the designated row element is not included in the count table, the MCU 30 may determine whether or not the designated row element includes a cell of a new column element not included in the count table. When it is determined that the designated row element includes a cell of a new column element, the MCU 30 adds the new column element to the new count table. On the other hand, when the designated row element is already included in the count table or the designated row element does not include a cell of a new column element, the column element is not added to the count table. As a result, duplication of the row elements and duplication of the column elements in a count table are suppressed, and the count table can be made compact.

### [Viewpoints A12 and A13]

The database may include a plurality of row elements in which a parent-child relationship is defined. The parent-child relationship may be a relationship in which a finished product is a parent and ingredients constituting the finished product are children. In some cases, it is necessary to count colonies in a culture result of the entire product (finished product) and to count colonies in culture results of individual ingredients constituting the product. Therefore, since the parent-child relationship is defined in advance, the user's man-hours at the time of creating the count table are reduced. For example, when a certain finished product (for example, sandwich) is selected, ingredients (for example, ham and lettuce) may be presented in a selectable manner.

### [Viewpoint A14]

The table management section (for example, the MCU 30) may collectively add the plurality of row elements in which the parent-child relationship is defined to a new count table. As a result, the burden on the user at the time of creating the count table may be further mitigated.

### [Viewpoint A15]

When application of statistical processing is instructed, the table management section (for example, the MCU 30) may create the count table to include n row elements respectively storing count results of n culture vessels, which culture the same test individual, and at least one row element storing statistical processing results of the n row elements. According to FIG. 11, a case of n=2 is described. Here, n may be three or more. This may make it easy to create the count table for executing the statistical processing such as averaging.

### [Viewpoint A16]

As illustrated in FIG. 16, the display control section (for example, the MCU 30) may cause the display device 37 to display the test image and the count table side by side (simultaneously in parallel). The cell identifying section (for example, the MCU 30) may identify the target cell according to selection of the user from among the plurality of cells included in the count table displayed on the display device 37 together with the test image. The display control section (for example, the MCU 30) may cause the display device to display the count table in which the number of the colonies counted by the counting section has been reflected on the target cell together with the test image. As a result, the cell in which the count result is stored can be easily selected.

### [Viewpoint A17]

As illustrated in FIG. 25, the display control section (for example, the MCU 30) may display a first control object (for example, the tab 123) for setting an illumination condition included in the test condition associated with the target cell and a second control object (for example, the tab 124) for setting a counting condition included in the test condition on the display device 37. As a result, the test condition associated with the cell can be easily changed. Moreover, when sensing that the first control object (for example, the tab 123) has been operated by the user, the display control section (for example, the MCU 30) may display a setting screen (for example, the confirmation screen 110) for setting the illumination condition on the display device. When sensing that the second control object (for example, the tab 124) has been operated by the user, the display control section (for example, the MCU 30) may display a setting screen (for example, the setting screen 120) for setting the counting condition on the display device.

### [Viewpoint A18]

The display control section (for example, the MCU 30) may display, on the display device 37, a third control object (for example, the first software button 105a) for instructing the counting section to execute counting and a fourth control object (for example, the second software button 105b) for instructing the counting section to register the count result in the target cell. As a result, the user can easily instruct the count and instruct the registration of the count result.

### [Viewpoint A19]

When sensing that the third control object has been operated by the user, the display control section (for example, the MCU 30) may assign the third control object from a control object (for example, the count button) for instructing counting to a control object (for example, the capture button or the re-capture button) for instructing the acquisition section to acquire the test image. That is, the MCU 30 may change a command issued by operating the third control object from a command for instructing the counting to a command for instructing the acquisition of the test image. As a result, the number of operable buttons is reduced, and the user can easily determine what needs to be operated now.

### [Viewpoint A20]

As illustrated in FIG. 18, when the third control object is assigned to the control object (for example, the count button) for instructing the counting, the display control section (for example, the MCU 30) may display the fourth control object (for example, the register button) so as not to be operable by the user. As illustrated in FIG. 19, when the third control object assigned to the control object for instructing the counting is operated, a counting result is acquired. When the counting result is acquired, the display control section (for example, the MCU 30) may assign the third control object to the control object (for example, the capture button) for instructing the acquisition section to acquire the test image, and change the fourth control object (for example, the register button) to be operated by the user. That is, when the command for instructing the counting is assigned to the third control object, the fourth control object may be displayed so as not to be operated by the user. Moreover, when the third control object to which the command for instructing the counting is assigned is operated and the count result is acquired, the command for instructing the acquisition section to acquire the test image may be assigned to the third control object, and the display of the fourth control object may be changed so as to be operated by the user. As a result, the user can easily determine what needs to be operated now.

### [Viewpoint A21]

As illustrated in FIGS. 19 and 20, when the fourth control object (for example, the register button) is operated, the display control section (for example, the MCU 30) may register the count result in the target cell and change the fourth control object again so as not to be operated by the user. Here, the cell identifying section changes the target cell to the next cell. As illustrated in FIGS. 20 and 18, when the third control object (for example, the capture button) to which the command for instructing the acquisition section to acquire the test image has been assigned is operated, the display control section (for example, the MCU 30) may cause the acquisition section to acquire the test image, and assign the command for instructing the counting to the third control object (for example, the count button). As a result, the user can easily determine what needs to be operated now.

### [Viewpoint A22]

The head device 1 may further include, for example, a first hardware button and a second hardware button provided on a housing of the colony counting device. The same function may be assigned to the first hardware button and the third control object, and the same function may be assigned to the second hardware button and the fourth control object. As a result, it is possible to link the hardware button and the software button. In a case where the user is gazing at the Petri dish 15 set in the head device 1, an instruction can be input by the hardware button of the head device 1. That is, the user can easily input the instruction without moving the line of sight to the display device 37 of the PC 1b and operating the pointing device 33. On the other hand, in a case where the user is gazing at the test image displayed on the PC 1b, shifting the line of sight to the hardware button and pressing the hardware button may reduce work efficiency. Therefore, in this case, the software button is displayed on the display device 37 so that the user can easily and accurately operate the button.

### [Viewpoint A23]

The application program 39 is an example of a program executed in a control device that controls a colony counting device. The application program 39 causes the PC 1b to execute:
storing a count table, which includes a cell to which a count result of each of a plurality of test individuals is input, in a storage section;
displaying the count table stored in the storage section on a display device;
identifying a target cell to which a count result is input from among a plurality of the cells included in the count table displayed on the display device;
acquiring a test image that is an image of the test individual;
acquiring the number of colonies included in the test individual based on the test image acquired by an acquisition section according to a counting instruction input by a user; and
reflecting the number of colonies on the target cell.

### [Viewpoint A24]

According to the above embodiment, a control method for controlling the colony counting device 1 is provided. The control method includes:
storing a count table, which includes a cell to which a count result of each of a plurality of test individuals is input, in a storage section;
displaying the count table stored in the storage section on a display device;
identifying a target cell to which a count result is input from among a plurality of the cells included in the count table displayed on the display device;
acquiring a test image that is an image of the test individual;
counting colonies included in the test individual based on the test image acquired by an acquisition section according to a counting instruction input by a user; and
reflecting the number of the counted colonies on the target cell.

### [Viewpoint B1]

The storage device 35 functions as a storage section that stores a count table and identification information associated with the count table that includes a cell to which a colony count result for a test individual is input. The MCU 30 and the MCU 20 function as an identification information acquisition section that acquires identification information from an identification image (for example, one-dimensional symbol or two-dimensional symbol) obtained by encoding the identification information. The MCU 30 functions as a table management section that reads the count table associated with the identification information acquired by the acquisition section from the storage section. Moreover, the MCU 30 functions as a cell identifying section that identifies a target cell to which a count result is input from among a plurality of the cells included in the count table read by the table management section. The MCU 20 or the MCU 30 functions as a counting instruction section that generates a counting instruction according to an operation of a user. The main camera 11 functions as a first imaging section that captures a test image that is an image of the test individual based on the counting instruction generated by the counting instruction section. The MCU 20 or the MCU 30 functions as a counting section that counts colonies included in the test individual based on the test image captured by the first imaging section. The table management section (for example, the MCU 30) is configured to reflect the number of the colonies counted by the counting section on the target cell identified by the cell identifying section. Since the count table to which the count result is input is identified from the identification image and displayed in this manner, the burden on the user regarding the counting of colonies is mitigated.

### [Viewpoint B2]

The identification information acquisition section may be configured to acquire the identification information from an identification image captured by the first imaging section (for example, the main camera 11). In this manner, an imaging section that captures an image of the test individual may also be used as an imaging section that captures the identification image.

### [Viewpoint B3]

The identification information acquisition section may include a second imaging section. The front camera 10 is an example of the second imaging section that captures an identification image. The identification information acquisition section (for example, the MCUs 20 and 30) may be configured to acquire the identification information from the identification image captured by the second imaging section.

### [Viewpoint B4]

The second imaging section (for example, the front camera 10) may be configured to capture an additional image that is at least one of an appearance of the test individual, an appearance of the test individual packaged by a packaging body (for example, a packaging bag or a product package), or information printed on the packaging body. The storage section (for example, the storage device 35) may store at least one of the additional image and additional information acquired from the additional image in association with the target cell. As described above, the count table may include the cell to which the count result is input and a cell (for example, a remark cell or a cell of a free column) capable of storing an image or the like. In this case, the additional image (for example, a product appearance) and the additional information (for example, a product code) may be stored in or be associated with the latter cell. The user can easily grasp which test individual has been used to obtain the count result by referring to the additional information or the additional image stored in association with the target cell.

### [Viewpoint B5]

The count table may include an additional cell (for example, the remark cell or the cell of the free column) that holds at least one of the additional image and the additional information. The user can easily grasp which test individual has been used to obtain the count result by referring to the additional information or the additional image held in the additional cell.

### [Viewpoint B6]

As illustrated in FIGS. 29 and 40, the MCU 30 and the pointer 57 may function as a selection section that selects one additional cell from a plurality of additional cells existing in the count table. Moreover, the MCU 30 may function as an additional information registration section that registers at least one of the additional image and the additional information in the one additional cell selected by the selection section. As a result, the user can register the additional image or the additional information to a desired cell in the count table.

### [Viewpoint B7]

The MCU 30 may function as an obtaining section that obtains identification information (for example, a sample name or a Petri dish number) of the test individual associated with the target cell. The storage section may be configured to store the test image of the test individual captured by the first imaging section according to the counting instruction input by the user in association with the identification information of the test individual. In the related art, a lot of man-hours are required to correctly record a relationship between the test image and the test individual. For example, it is conceivable to acquire a test image with a digital camera, but in this case, it may be necessary to manually associate the test image with identification information of the test individual. Further, the manual association causes human error. In the present embodiment, the MCU 30 identifies the target cell, and associates the test image of the test individual with the identification information of the test individual that is associated with the target cell. Therefore, the number of terms required by the user is reduced as compared with the related art, and the relationship between the test image of the test individual and the identification information of the test individual can be correctly recorded.

### [Viewpoint B8]

As illustrated in FIG. 33, the MCU 30 may function as a report generation section that generates a report including the identification information of the test individual, the number of colonies counted by the counting section, and the test image of the test individual. As a result, the user can intuitively grasp which sample has been measured to obtain the result.

### [Viewpoint B9]

Unique cell identification information may be given to a cell to which the number of colonies is input in the count table. As illustrated in FIG. 41, the MCU 30 may print the identification image 221 obtained by encoding the unique cell identification information on the seal 270 (resin or paper having an adhesive surface) by the printer 38. The user attaches the seal 270 to a side surface of the Petri dish 15. Note that the Petri dish number may be used as the unique cell identification information. The identification information acquisition section (for example, the MCU 30, the front camera 10, or the main camera 11) may be configured to acquire the cell identification information. The cell identifying section (for example, the MCU 30) may identify the target cell based on the cell identification information acquired by the identification information acquisition section. As a result, the user can save time and effort for designating the target cell. Moreover, the target cell corresponding to the test individual may be accurately identified.

### [Viewpoint B10]

As illustrated in FIG. 28, the identification information acquisition section (for example, the MCU 30) may acquire user authentication information by the first imaging section or the second imaging section. As a result, a dedicated camera and a code reader for user authentication can be omitted.

### [Viewpoint B11]

As illustrated in FIG. 27, the identification information acquisition section (for example, the MCU 30, the front camera 10, or the main camera 11) may be configured to acquire the identification information from an identification image printed on a print medium (for example, the test list 220).

### [Viewpoint B12]

The MCU 30 may function as a data creation section that creates data of a test list including s count table and identification information associated with the count table.

### [Viewpoint B13]

The identification information acquisition section (for example, the MCU 30, the front camera 10, or the main camera 11) may be configured to acquire the identification information from an identification image displayed on the terminal device 1c. As a result, it is possible to reduce paper media.

### [Viewpoint B14]

The communication circuit 34 is an example of a communication section that communicates with the terminal device 1c and transmits the identification image to the terminal device 1c.

### [Viewpoint B15]

The MCU 30 may function as a creation section that creates a count table in accordance with a user operation and causes the storage section to store the count table.

### [Viewpoint B16]

The creation section (for example, the MCU 30) may associate a test condition with a cell storing the count result of the number of colonies. The first imaging section may be configured to capture an image of the test individual according to the test condition (for example, an exposure time, an illumination type, or brightness).

### [Viewpoint B17]

The colony counting device 1 may further include a housing (for example, the upper unit 2, the support unit 3, and the lower unit 4) having a first imaging section.

The housing may include: the stage 5 that holds the Petri dish 15 accommodating a test individual; an illumination section (for example, the ring illumination devices 12 and 13 or the coaxial illumination device 14) that illuminates the test individual; and a receiving section (for example, the first hardware button 8a) that receives a counting instruction input by a user.

### [Viewpoint B18]

The colony counting device 1 may further include a housing (for example, the upper unit 2, the support unit 3, and the lower unit 4) including a first imaging section and a second imaging section. The housing may include: the stage 5 that holds the Petri dish 15 accommodating a test individual; an illumination section (for example, the ring illumination devices 12 and 13 or the coaxial illumination device 14) that illuminates the test individual; and a receiving section (for example, the first hardware button 8a) that receives a counting instruction input by a user. Moreover, the housing may have the recess 4a. The second imaging section (for example, the front camera 10) may be arranged in the recess. The receiving section (for example, the first hardware button 8a) may be arranged in an operation section (the operation section 8) between the stage and the recess. As a result, it is possible to easily input the counting instruction.

### [Viewpoint B19]

When the receiving section (for example, the first hardware button 8a) receives an imaging instruction when the colony counting device 1 is in a first state, the first imaging section may execute imaging. When the receiving section receives the imaging instruction when the colony counting device 1 is in a second state different from the first state, the second imaging section may execute imaging. As a result, it is possible to instruct the different imaging sections to execute the imaging even though the same operation is performed on the single receiving section. The first state is, for example, a state in which a count table has already been identified. The second state is, for example, a state in which a count table has not yet been identified.

### [Viewpoint B20]

A program executed in a processor that controls a colony counting device, the program causing the processor to execute:
causing a storage section to store a count table and identification information associated with the count table that includes a cell to which a colony count result for a test individual is input;
acquiring the identification information from an identification image obtained by encoding the identification information;
reading the count table associated with the acquired identification information from the storage section;
identifying a target cell to which a count result is input from among a plurality of the cells included in the read count table;
generating a counting instruction according to an operation of a user;
capturing a test image that is an image of the test individual based on the generated counting instruction;
counting colonies included in the test individual based on the captured test image; and
reflecting the number of the counted colonies on the identified target cell.

### [Viewpoint B23]

A control method executed in a processor that controls a colony counting device, the control method including:
storing a count table and identification information associated with the count table in a storage section, the count table including a cell to which a colony count result for a test individual is input;
acquiring the identification information from an identification image obtained by encoding the identification information;
reading the count table associated with the acquired identification information from the storage section;
identifying a target cell to which a count result is input from among a plurality of the cells included in the read count table;
generating a counting instruction according to an operation of a user;
capturing a test image that is an image of the test individual based on the generated counting instruction;
counting colonies included in the test individual based on the captured test image; and
reflecting the number of the counted colonies on the identified target cell.

### [Viewpoint C]

[Viewpoint C1]

A colony counting device including:
a stage (for example, the transmission window 6) that has a first surface on which a test individual is placed and a second surface, which is a back surface of the first surface, and is capable of transmitting light from the second surface to the first surface;
an imaging section (for example, the main camera 11) that is arranged to oppose the first surface of the stage and generates a test image of the test individual placed on the stage;
a surface light source (for example, the coaxial illumination device 14) that irradiates the second surface with test light such that a beam of the test light is transmitted from the second surface of the stage to the first surface;
a dimming section (for example, the diffusion plates 13c and 13d) that is arranged between the surface light source and the second surface of the stage and adjusts a degree of diffusion of the beam of the test light;
a control section (for example, the MCU 20 or 30, or the dimming control section 13z) that controls at least the dimming section; and
a counting section (for example, the MCU 20 or 30) that counts the number of colonies included in the test image generated by capturing, by the imaging section, the test individual irradiated with the beam of the test light output from the surface light source and having the degree of diffusion adjusted by the dimming section.

According to the present embodiment, since the dimming section can adjust the degree of diffusion, burden on the user for switching the degree of diffusion is mitigated. Further, it is possible to select an optimal illumination (degree of diffusion) according to types of bacteria species and culture medium to be cultured, and it is possible to improve the counting accuracy.

### [Viewpoint C2]

The colony counting device according to Viewpoint C1, wherein the surface light source includes a light distribution angle regulating section (for example, the light distribution regulating plate 4301) that regulates a light distribution angle of the test light output from the surface light source.

In general, each light emitting element is independent in the surface light source, and thus, unevenness in the amount of light occurs. In particular, the test individual may be irradiated with parallel light from the surface light source as the test light. In order to reduce the unevenness in the amount of light, it is necessary to move the surface light source away from the transmission window 6, which leads to an increase in a size of the head device 1a. Therefore, the light distribution angle regulating section is adopted so that the unevenness in the amount of light is reduced even if a distance from the surface light source to the transmission window 6 is reduced.

### [Viewpoint C3]

The colony counting device according to aspect C2, wherein
the light distribution angle regulating section includes:
a plurality of light guides (for example, the light guides 4302) arranged for a plurality of light emitting elements (for example, the light emitting elements 14a), respectively, which form the surface light source; and
a plurality of apertures (for example, the apertures 4303) arranged for the plurality of light guides, respectively.

Since such a structure is adopted, a desired light distribution angle is achieved, and the unevenness in the amount of light is reduced. Further, an optical system can be configured in a smaller size while maintaining the area of irradiation as compared with a configuration including a single light source and a lens optical system.

### [Viewpoint C4]

The colony counting device according to aspect C3, wherein the plurality of light emitting elements are arranged on any one of a plurality of concentric circles having different radii (for example, FIG. 43).

Since the plurality of light emitting elements are arranged in this manner, the unevenness in the amount of light is further reduced.

### [Viewpoint C5]

The colony counting device according to Viewpoint C4, wherein an arrangement interval (for example, i1) between a plurality of light emitting elements arranged on a concentric circle having a larger radius among the plurality of concentric circles is narrower than an arrangement interval (for example, i2) between a plurality of light emitting elements arranged on a concentric circle having a smaller radius among the plurality of concentric circles (for example, i2>i1 in FIG. 43).

Since the plurality of light emitting elements are arranged in this manner, a difference between the amount of light at the central portion and the amount of light at the outer edge decreases in the surface light source.

### [Viewpoint C6]

The colony counting device according to Viewpoint C1, wherein the light distribution angle regulating section includes a telecentric lens (for example, the telecentric optical system 4802) arranged between the surface light source and the dimming section.

In this manner, the light distribution angle regulating section may be achieved by the telecentric lens.

### [Viewpoint C7]

The colony counting device according to Viewpoint C1, wherein
the dimming section is capable of switching the degree of diffusion between a first degree of diffusion and a second degree of diffusion, and
the first degree of diffusion is greater than the second degree of diffusion.

In this manner, the dimming section capable of achieving at least two degrees of diffusion may be adopted. As a result, the user can easily select the degree of diffusion.

### [Viewpoint C8]

The colony counting device according to Viewpoint C7, wherein the imaging section generates a test image in which a scratch on a container (for example, the Petri dish 15) accommodating the test individual or noise caused by the test individual is reduced when the degree of diffusion of the beam of the test light is switched to the first degree of diffusion by the dimming section.

When the degree of diffusion is increased, it is possible to reduce the scratch on the container containing the test individual and the noise caused by the test individual, and it is possible to accurately execute a count.

### [Viewpoint C9]

The colony counting device according to Viewpoint C7, wherein the imaging section generates a test image in which contours of the colonies are emphasized when the degree of diffusion of the beam of the test light is switched to the second degree of diffusion by the dimming section.

The MCU 30 may measure circularity, an aspect ratio, area, a circumferential length, and the like of a colony according to a test condition. In this case, the second degree of diffusion with which the contours of the colonies are emphasized will be effective.

### [Viewpoint C10]

The colony counting device according to Viewpoint C1, further including a storage section (for example, the storage device 35) that stores the degree of diffusion set in the dimming section when the test image is acquired and the test image in association with each other.

The degree of diffusion is one of the test conditions 28. As illustrated in FIG. 4, the test conditions 28 are stored in the storage device 35 in association with the count table 55 and the test image. That is, the degree of diffusion set in the dimming section when the test image 29 is acquired and the test image 29 are stored in the storage device 35 in association with each other.

### [Viewpoint C11]

The colony counting device according to Viewpoint C10, wherein
the control section is configured to control the imaging section to acquire a first test image (for example, the test image 5301 or 5501 with a high degree of diffusion) as the test image, which is a counting target of the counting section, in a state in which the dimming section is controlled to irradiate the test individual with test light with a first degree of diffusion, and to control the imaging section to acquire a second test image (for example, the test image 5302 or 5502 with a low degree of diffusion) in a state in which the dimming section is controlled to irradiate the test individual with test light with a second degree of diffusion lower than the first degree of diffusion,
the colony counting device further including:
a registration section that registers the first test image and degree-of-diffusion information related to the first degree of diffusion in the storage section in association with each other, and registers the second test image and degree-of-diffusion information related to the second degree of diffusion in the storage section in association with each other; and
an image processing section that calculates a difference between the first test image and the second test image to generate a disturbance-suppressed image in which influence of disturbance observed as a bright area in both the first test image and the second test image is suppressed, and
the counting section counts the number of colonies based on the disturbance-suppressed image generated by the image processing section as the test image.

The MCU 30 functions as the registration section that registers the test images 5301 and 5501 and the degree-of-diffusion information related to the first degree of diffusion in the storage device 35 in association with each other. Moreover, the MCU 30 functions as the registration section that registers the second test images 5302 and 5502 and the degree-of-diffusion information related to the second degree of diffusion in the storage device 35 in association with each other. The degree-of-diffusion information may include a numerical value, a level, a degree (strong/weak, large/small, or high/low), a function (on/off of noise reduction), and the like.

### [Viewpoint C12]

The colony counting device according to Viewpoint C1, wherein
the dimming section includes a diffusion member (for example, the diffusion plate 13c, or the diffusion film 4901 or 4902 of the liquid crystal film type) capable of electrically changing the degree of diffusion, and
the control section changes the degree of diffusion of the dimming section by electrically changing a degree of diffusion of the diffusion member.

This makes it possible to immediately switch the degree of diffusion. Further, an installation space of the diffusion member can be reduced.

### [Viewpoint C13]

The colony counting device according to Viewpoint C1, wherein
the dimming section includes a plurality of diffusion members (for example, the diffusion plate 13c, and the diffusion films 4901 and 4902 of the liquid crystal film type) capable of electrically changing the degree of diffusion, and
the control section changes the degree of diffusion of the dimming section by electrically changing degrees of diffusion of the plurality of diffusion members.

In the above embodiment, the diffusion plate 13c is achieved by the diffusion films 4901 and 4902, but the diffusion plate 13d may also be achieved by the diffusion films 4901 and 4902. That is, the diffusion plate 13d may also be capable of switching the degree of diffusion similarly to the diffusion plate 13c.

### [Viewpoint C14]

The colony counting device according to Viewpoint C12 or 13, wherein the control section (for example, the dimming control section 13z) changes the degree of diffusion of the diffusion member by changing a voltage applied to the diffusion member.

Since the degree of diffusion can be changed by changing an electrical parameter in this manner, the dimming section and the control section can be easily mounted.

### [Viewpoint C15]

The colony counting device according to Viewpoint C1, wherein
the dimming section includes:
a transparent substrate having translucency (for example, the base material 4900 such as a glass plate);
a first diffusion film (for example, the diffusion film 4901 or 4902) provided on a first surface of the transparent substrate; and
a second diffusion film (for example, the diffusion film 4901 or 4902) provided on a second surface of the transparent substrate, and
the control section (for example, the dimming control section 13z) electrically changes a degree of diffusion of at least one of the first diffusion film and the second diffusion film to change the degree of diffusion of the dimming section.

Since the degrees of diffusion of the plurality of diffusion films can be selectively changed in this manner, a plurality of degrees of diffusion can be achieved. Further, among the plurality of diffusion films, the MCU 30 may preferentially use a diffusion film capable of further reducing the unevenness in the amount of light.

### [Viewpoint C16]

The colony counting device according to Viewpoint C1, wherein
the dimming section includes:
a diffusion member (for example, the diffusion plate 13c or 13d) having a predetermined degree of diffusion; and
an actuator (for example, the motor 13e) that moves the diffusion member so as to arrange the diffusion member in an optical path of the test light and retract the diffusion member from the optical path, and
the control section (for example, the dimming control section 13z) controls the actuator to change a degree of diffusion of the beam of the test light.

In a case where there is a margin in the size of the head device 1a, such a mechanism that mechanically adjusts the degree of diffusion may be adopted. As a result, the user can easily switch the degree of diffusion.

### [Viewpoint C17]

The colony counting device according to Viewpoint C1, further including:
a setting receiving section (for example, the MCU 30, the UI 5400, or the check box 5403) that receives a setting of a degree of diffusion for each of types of culture media; and
a type receiving section (for example, the MCU 30 or the dialog 90) that receives an input of a type of a culture medium of the test individual placed on the stage,
wherein the control section is configured to identify a degree of diffusion corresponding to the type of the culture medium of the test individual received by the type receiving section and control the dimming section according to the identified degree of diffusion.

In this manner, the MCU 30 may receive the degree of diffusion for each culture medium type through the keyboard 32 or the pointing device 33, and store the culture medium type and the degree of diffusion in association with each other in the storage device 35. Moreover, the MCU 30 may receive the input of the type of the culture medium of the test individual placed on the stage through the keyboard 32 or the pointing device 33. The MCU 30 may identify the degree of diffusion corresponding to the type of the culture medium of the test individual received by the type receiving section, and control the diffusion plate 13c according to the identified degree of diffusion.

### [Viewpoint C18]

The colony counting device according to Viewpoint C1, further including
a creation section (for example, the MCU 30) that creates a count table in which identification information for identifying the test individual, degree-of-diffusion information related to the degree of diffusion of the dimming section applied to the test individual, and the number of colonies acquired by the counting section are recorded,
wherein the control section is configured to receive selection of a cell from the count table and control the degree of diffusion of the dimming section based on the degree-of-diffusion information corresponding to the received cell, and
the counting section is configured to write the number of colonies in the received cell.

In this manner, the MCU 30 functions as the creation section that creates the count table in which the identification information for identifying the test individual, the degree-of-diffusion information related to the degree of diffusion of the dimming section applied to the test individual, and the number of colonies acquired by the counting section are recorded. The MCU 30 may be configured to control the degree of diffusion of the dimming section based on the degree-of-diffusion information of the count table. Moreover, the MCU 30 writes the number of colonies in the count table.

### [Viewpoint C19]

The colony counting device according to Viewpoint C1, further including a calculation section (for example, the MCU 30) that calculates, from the test image, a size (for example, the diameter Dc) of an inhibition halo formed around bacteria as a drug spreads,
wherein the test individual includes a container, a culture medium (for example, the culture medium 5601) which is accommodated in the container and to which the bacteria are applied, and the drug (for example, the drug 5602) placed on the culture medium.

As illustrated in FIG. 56, the colony counting device 1 may be used to measure the inhibition halo 5603. Conventionally, the inhibition halo 5603 has been measured using calipers or the like, but the inhibition halo 5603 can be easily measured using the colony counting device 1 in the present embodiment.

### [Viewpoint C20]

A control method for a colony counting device, which includes:
a stage that has a first surface on which a test individual is placed and a second surface, which is a back surface of the first surface, and is capable of transmitting light from the second surface to the first surface;
an imaging section that is arranged to oppose the first surface of the stage and generates a test image of the test individual placed on the stage;
a surface light source that irradiates the second surface with test light such that a beam of the test light is transmitted from the second surface of the stage to the first surface; and
a dimming section that is arranged between the surface light source and the second surface of the stage and adjusts a degree of diffusion of the beam of the test light,
the control method including:
setting the degree of diffusion to the dimming section;
turning on the surface light source;
generating a test image by capturing, by the imaging section, the test individual irradiated with the beam of the test light that is output from the surface light source, is adjusted in the degree of diffusion by the dimming section, enters through the second surface of the stage, and exits through the first surface of the stage; and
counting the number of colonies included in the test image.

### [Viewpoint C21]

A program that causes a colony counting device to execute the control method according to Viewpoint C20.

### [Viewpoint C22]

A colony counting device including:
a setting receiving section (for example, the MCU 30, the UI 5400, or the check box 5403) that receives a setting of a degree of diffusion for each of types of culture media;
a stage on which a test individual is placed;
a type receiving section (for example, the MCU 30 or the dialog 90) that receives an input of a type of a culture medium of the test individual placed on the stage;
an illumination section that emits test light to the test individual placed on the stage;
a dimming section provided in an optical path through which the test light emitted from the illumination section reaches the test individual, the dimming section adjusting a degree of diffusion of the test light emitted from the illumination section;
a control section (for example, the MCU 20, the MCU 30, or the dimming control section 13z) that identifies a degree of diffusion corresponding to the type of the test individual received by the type receiving section and controls the dimming section according to the identified degree of diffusion;
an imaging section that receives the test light, which has been emitted from the illumination section, transmitted through the dimming section, and transmitted through the test individual placed on the stage or reflected by the test individual, and generates a test image of the test individual; and
a counting section that counts the number of colonies included in the test image generated by the imaging section for the test individual irradiated with the test light via the dimming section.

### [Viewpoint D]

[Viewpoint D1]

A colony counting device including:
an acquisition section (for example, the MCU 30 or the main camera 11) that acquires an image of a test individual obtained by capturing a test individual;
a display processing section (for example, the MCU 30 or the display control circuit 36) that displays a plurality of colony detection results, obtained by applying different colony detection parameters to the image of the test individual acquired by the acquisition section, on a display section in a comparable manner;
a selection section (for example, the MCU 30 or the pointing device 33) that selects one colony detection result from among the plurality of colony detection results displayed on the display section according to an operation of a user; and
a counting section (for example, the MCU 30) that applies the colony detection parameter used to obtain the one colony detection result selected by the selection section to the image of the test individual and counts the number of colonies included in the image of the test individual.

According to Viewpoint D1, it is easy to adjust test parameters in the colony counting device 1. That is, the user can indirectly select the colony detection parameter by comparing the plurality of colony detection results and selecting one colony detection result that suits his/her sense. Therefore, it is easy to adjust the colony detection parameters. Note that the selection section may select one colony detection result according to an operation of the user from among the plurality of colony detection results displayed simultaneously on the display section or displayed while being sequentially switched one by one. In either case, it can be said that the plurality of colony detection results are displayed in a comparable manner.

### [Viewpoint D2]

The colony counting device according to Viewpoint D1, wherein each of the plurality of colony detection results is an image in which a marker indicating a colony detection position is superimposed on the image (for example, an original image or a binarized image) of the test individual.

As a result, the user can confirm an image area that the user considers as a colony while viewing the image of the test individual.

Each of the plurality of colony detection results may include a marker (for example, a cross mark or a binarized image superimposed on an original image) indicating the colony detection position.

As a result, the user can confirm whether or not the image area that the user considers as the colony is extracted as a colony while viewing the image of the test individual. That is, the user can adjust the parameters only by confirming the image without looking at values of the parameters.

### [Viewpoint D3]

The colony counting device according to Viewpoint D1 or D2, wherein each of the plurality of colony detection results includes a numerical value (for example, a count number) indicating the number of detected colonies.

As a result, the user can compare the number of colonies counted by himself/herself with the number of colonies obtained by the colony counting device 1. As a result, the user can adjust the parameters only by confirming the number of colonies without viewing the values of the parameters.

### [Viewpoint D4]

The colony counting device according to any one of Viewpoints D1 to D3, wherein each of the plurality of colony detection results are displayed on the display section in association with the colony detection parameter used to detect colonies.

As a result, it will be possible to learn which value of the colony detection parameter is appropriate.

### [Viewpoint D5]

The colony counting device according to any one of Viewpoints D1 to D4, further including:
an image processing section (for example, the MCU 30) that generates a binarized image from the image of the test individual based on the colony detection parameter; and
a detection section (for example, the MCU 30) that detects colonies from the binarized image,
wherein the counting section counts the number of the colonies detected by the detection section, and
the plurality of colony detection results include the binarized image, detection positions of the colonies, and the number of the colonies.

As illustrated in FIG. 60 and the like, the colony detection result may include the binarized image, the detection positions of the colonies, and the number of the colonies. This will make it easier for the user to select a colony detection result that is close to his/her sense.

### [Viewpoint D6]

The colony counting device according to any one of Viewpoints D1 to D5, further including
a determining section (for example, the MCU 30, S121) that determines a plurality of different colony detection parameters to serve as secondary candidates based on the colony detection parameter used to obtain the one colony detection result when the one colony detection result is selected from among the plurality of colony detection results acquired using the different colony detection parameters to serve as primary candidates,
wherein, when the determining section determines the plurality of different colony detection parameters serving as the secondary candidates,
the display processing section displays a plurality of colony detection results, obtained by applying the plurality of different colony detection parameters serving as the secondary candidates to the image of the test individual, in a comparable manner on the display section (for example, S122, S123),
the selection section selects one colony detection result according to an operation of the user from among the plurality of colony detection results simultaneously displayed on the display section and obtained by applying the plurality of different colony detection parameters serving as the secondary candidates (for example, S124), and
the counting section applies the colony detection parameter used to obtain the one colony detection result selected by the selection section to the image of the test individual and counts the number of colonies included in the image of the test individual.

In this manner, the colony detection parameters serving as the secondary candidates may be determined from the colony detection parameters serving as the primary candidates. As a result, the colony detection parameters may be narrowed down stepwise.

### [Viewpoint D7]

The colony counting device according to Viewpoint D6, wherein
the determining section determines a plurality of different colony detection parameters to serve as tertiary candidates based on the colony detection parameter used to obtain the one colony detection result when the one colony detection result is selected from among the plurality of colony detection results acquired using the different colony detection parameters and serving as the secondary candidates,
when the determining section determines the plurality of different colony detection parameters serving as the tertiary candidates,
the display processing section displays a plurality of colony detection results, obtained by applying the plurality of different colony detection parameters serving as the tertiary candidates to the image of the test individual, in a comparable manner on the display section,
the selection section selects one colony detection result according to an operation of the user from among the plurality of colony detection results displayed on the display section and obtained by applying the plurality of different colony detection parameters serving as the tertiary candidates, and
the counting section applies the colony detection parameter used to obtain the one colony detection result selected by the selection section to the image of the test individual and counts the number of colonies included in the image of the test individual.

In this manner, the colony detection parameters serving as the tertiary candidates may be determined from the colony detection parameters serving as the secondary candidates. As a result, the colony detection parameters may be narrowed down stepwise.

### [Viewpoint D8]

The colony counting device according to Viewpoint D6, wherein an interval between the plurality of colony detection parameters serving as the secondary candidates is finer than an interval between the plurality of colony detection parameters serving as the primary candidates.

This will allow the colony detection parameters to be adjusted gradually and precisely.

### [Viewpoint D9]

The colony counting device according to Viewpoint D6 or D7, wherein
the plurality of colony detection parameters serving as the secondary candidates include:
a first colony detection parameter corresponding to the one colony detection result selected by the selection section from among the plurality of colony detection parameters serving as the primary candidates;
a second colony detection parameter larger than the first colony detection parameter; and
a third colony detection parameter smaller than the first colony detection parameter.

In this manner, the plurality of colony detection parameters serving as the secondary candidates may include the colony detection parameter selected from among the primary candidates. This allows the user to gradually approach the colony detection parameter close to his/her sense.

### [Viewpoint D10]

The colony counting device according to Viewpoint D6, further including
a calculation section (for example, the MCU 30) that calculates the plurality of colony detection results using the different colony detection parameters,
wherein the calculation section calculates a plurality of colony detection results in advance using a plurality of colony detection parameters (for example, three sensitivities out of five sensitivities) serving as primary candidates and a plurality of colony detection parameters (for example, two sensitivities out of the five sensitivities) serving as secondary candidates, and stores the plurality of colony detection results in a storage section (for example, the storage device 35),
the display processing section reads a plurality of colony detection results obtained using the plurality of colony detection parameters serving as the primary candidates from the storage section and displays the plurality of read colony detection results on the display section,
the selection section selects one colony detection result according to an operation of the user from among the plurality of colony detection results obtained by applying the plurality of colony detection parameters serving as the primary candidates and displayed on the display section,
the display processing section reads the one colony detection result and a plurality of colony detection results obtained using the plurality of colony detection parameters serving as the secondary candidates from the storage section and displays the read one colony detection result and the plurality of read colony detection results on the display section,
the selection section selects one colony detection result according to an operation of the user from among the one colony detection result and the plurality of colony detection results, obtained by applying the plurality of colony detection parameters serving as the secondary candidate, displayed on the display section, and
the counting section applies the colony detection parameter used to obtain the one colony detection result selected by the selection section to the image of the test individual and counts the number of colonies included in the image of the test individual.

In this manner, the colony detection parameter selected from the primary candidates may be substantially treated as one colony detection parameter of the secondary candidates.

### [Viewpoint D11]

The colony counting device according to any one of Viewpoints D1 to D10, wherein
when a first type colony detection parameter (for example, sensitivity) is decided and stored in a saving section in response to an operation of the user,
the display processing section displays a plurality of colony detection results obtained by applying different second type colony detection parameters (for example, on/off of noise reduction processing) to the image of the test individual in a comparable manner while applying the first type colony detection parameter in common to the image of the test individual,
the selection section selects one colony detection result according to an operation of the user from among the plurality of colony detection results obtained by applying the different second type colony detection parameters and displayed on the display section, and
the counting section applies the first type colony detection parameter and the second type colony detection parameter used to obtain the one colony detection result selected by the selection section to the image of the test individual and counts the number of colonies included in the image of the test individual.

When the first type colony detection parameter is decided in this manner, adjustment processing is executed for the second type colony detection parameters. Also for the second type colony detection parameters, the plurality of colony detection results are displayed in a comparable manner. This allows the user to easily adjust the second type colony detection parameters as well.

### [Viewpoint D12]

The colony counting device of Viewpoint D11, wherein the first type colony detection parameter is any of
a binarization sensitivity serving as a reference when a binarized image used to detect the colonies from the image of the test individual is generated,
on/off of shading correction applied to the image of the test individual, and
an intensity (for example, a size of a small particle or a lint reduction effect) of noise reduction processing applied to the image of the test individual.

In this manner, a parameter that takes time to calculate may be adjusted first.

### [Viewpoint D13]

The colony counting device of Viewpoint D11 or D12, wherein the second type colony detection parameter is any of
on/off of reduction processing of reducing a small particle from the image of the test individual,
on/off of reduction processing of reducing an abnormal object (for example, lint) from the image of the test individual, and
on/off of reduction processing of reducing a large particle from the image of the test individual.

In this manner, a parameter that does not take time to calculate may be adjusted later.

### [Viewpoint D14]

The colony counting device according to any one of Viewpoints D1 to D14, wherein
the display processing section is configured to display a first screen (for example, the UI 5800 in FIG. 58) and a second screen (for example, the UI 5800 in FIG. 61 or the UI 6300 in FIG. 63) on the display section,
the first screen is a screen that displays the plurality of colony detection results, and
the second screen is a screen for adjusting another colony detection parameter different from the colony detection parameter corresponding to the one colony detection result selected by the operation of the user on the first screen.

Since the plurality of screens are switched in this manner, the amount of information included in one screen is reduced, and a user interface that is easy for the user to understand is achieved.

### [Viewpoint D15]

The colony counting device according to Viewpoint D15, wherein the colony detection parameter adjusted through the first screen (for example, the UI 5800) is adjusted by selecting the one colony detection result from among the plurality of colony detection results obtained by applying discretely adjusted image processing parameters (for example, the binarization sensitivity).

Since the image processing parameters are adjusted from discrete values in this manner, it will be easy to adjust consecutively changing image processing parameters on a limited display screen.

### [Viewpoint D16]

The colony counting device according to Viewpoint D14 or D15, wherein the second screen (for example, the UI 6300) is a screen for receiving adjustment of colony detection parameters (for example, sizes of small particles or lint reduction effects) having consecutive values.

In this manner, an adjustment screen of the colony detection parameters having consecutive values may be made easy.

### [Viewpoint D17]

The colony counting device according to any one of Viewpoints D1 to D17, wherein
the display processing section is configured to cause the display section to display a result display screen (for example, the UI 100) including:
the one colony detection result (for example, the test image 103) selected by the selection section according to the operation of the user;
identification information (for example, an ID or a Petri dish number) of the test individual corresponding to the one colony detection result;
a culture condition (for example, a culture medium, a dilution factor, and a culture time) for the test individual; and
the number of colonies (for example, a count number).

This allows the user to visually confirm an adjustment result.

### [Viewpoint D18]

The colony counting device according to Viewpoint D17, wherein
the display processing section displays an adjustment screen, which indicates the plurality of colony detection results in a comparable manner, on the display section,
the display processing section transitions from the adjustment screen to the result display screen when the colony detection parameter used to obtain the one colony detection result selected by the selection section through the adjustment screen is stored in a saving section,
the display processing section transitions from the result display screen to the adjustment screen when a transition to the adjustment screen is instructed on the result display screen, and
the selection section receives re-adjustment of the colony detection parameter through the adjustment screen.

As described in relation with FIG. 62, the MCU 30 may transition from the UI 100 to the UI 5800 again to receive re-adjustment of the colony detection parameters.

### [Viewpoint D19]

A control method for a colony counting device, the control method including:
acquiring an image of a test individual obtained by capturing the test individual;
displaying a plurality of colony detection results obtained by applying different colony detection parameters to the acquired image of the test individual in a comparable manner on a display section;
selecting one colony detection result from among the plurality of colony detection results displayed on the display section according to an operation of a user; and
applying the colony detection parameter used to obtain the selected one colony detection result to the image of the test individual and counting the number of colonies included in the image of the test individual.

In this manner, the control method is also a part of the present embodiment.

### [Viewpoint D20]

A program for causing a processor to execute the control method for a colony counting device according to Viewpoint D19.

Note that the program may be a computer program stored in the storage device 35.

### [Viewpoint D21]

A control method for a colony counting device, the control method including:
a first step of displaying a plurality of colony detection results, obtained by applying different first type colony detection parameters to an image of a test individual acquired by an acquisition section, on a display section in a comparable manner, selecting one colony detection result according to an operation of the user from among the plurality of colony detection results displayed on the display section, and storing a first type colony detection parameter used to obtain the selected one colony detection result in a storage device; and
a second step of displaying a plurality of colony detection results, obtained by applying the first type colony detection parameter applied to the one colony detection result and different second type colony detection parameters to the image of the test individual acquired by the acquisition section, on the display section in a comparable manner, selecting one colony detection result according to an operation of the user from among the plurality of colony detection results displayed on the display section, and storing the second type colony detection parameter used to obtain the selected one colony detection result in the storage device.

In this manner, a plurality of types of colony detection parameters may be adjusted through two steps. This allows the user to easily adjust the plurality of types of colony detection parameters.

The invention is not limited to the above embodiment, and various modifications and changes can be made within a scope of a gist of the invention.

1. A colony counting device comprising:
an acquisition section that acquires an image of a test individual obtained by capturing the test individual;
a display processing section that displays a plurality of colony detection results, obtained by applying different colony detection parameters to the image of the test individual acquired by the acquisition section, on a display section in a comparable manner;
a selection section that selects one colony detection result from among the plurality of colony detection results displayed on the display section according to an operation of a user; and
a counting section that applies the colony detection parameter used to obtain the one colony detection result selected by the selection section to the image of the test individual and counts a number of colonies included in the image of the test individual.

2. The colony counting device according to claim 1, wherein each of the plurality of colony detection results is an image in which a marker indicating a colony detection position is superimposed on the image of the test individual.

3. The colony counting device according to claim 1, wherein each of the plurality of colony detection results includes a numerical value indicating a number of detected colonies.

4. The colony counting device according to claim 1, wherein each of the plurality of colony detection results are displayed on the display section in association with the colony detection parameter used to detect colonies.

5. The colony counting device according to claim 1, further comprising:
an image processing section that generates a binarized image from the image of the test individual based on the colony detection parameter; and
a detection section that detects colonies from the binarized image,
wherein the counting section counts a number of the colonies detected by the detection section, and
the plurality of colony detection results include the binarized image, detection positions of the colonies, and the number of the colonies.

6. The colony counting device according to claim 1, further comprising
a determining section that determines a plurality of different colony detection parameters to serve as secondary candidates based on the colony detection parameter used to obtain the one colony detection result when the one colony detection result is selected from among the plurality of colony detection results acquired using the different colony detection parameters to serve as primary candidates,
wherein, when the determining section determines the plurality of different colony detection parameters serving as the secondary candidates,
the display processing section displays a plurality of colony detection results, obtained by applying the plurality of different colony detection parameters serving as the secondary candidates to the image of the test individual, in a comparable manner on the display section,
the selection section selects one colony detection result according to an operation of the user from among the plurality of colony detection results displayed on the display section and obtained by applying the plurality of different colony detection parameters serving as the secondary candidates, and
the counting section applies the colony detection parameter used to obtain the one colony detection result selected by the selection section to the image of the test individual and counts a number of colonies included in the image of the test individual.

7. The colony counting device according to claim 6, wherein
the determining section determines a plurality of different colony detection parameters to serve as tertiary candidates based on the colony detection parameter used to obtain the one colony detection result when the one colony detection result is selected from among the plurality of colony detection results acquired using the different colony detection parameters and serving as the secondary candidates,
when the determining section determines the plurality of different colony detection parameters serving as the tertiary candidates,
the display processing section displays a plurality of colony detection results, obtained by applying the plurality of different colony detection parameters serving as the tertiary candidates to the image of the test individual, in a comparable manner on the display section,
the selection section selects one colony detection result according to an operation of the user from among the plurality of colony detection results displayed on the display section and obtained by applying the plurality of different colony detection parameters serving as the tertiary candidates, and
the counting section applies the colony detection parameter used to obtain the one colony detection result selected by the selection section to the image of the test individual and counts a number of colonies included in the image of the test individual.

8. The colony counting device according to claim 6, wherein an interval between the plurality of colony detection parameters serving as the secondary candidates is finer than an interval between the plurality of colony detection parameters serving as the primary candidates.

9. The colony counting device according to claim 6, wherein
the plurality of colony detection parameters serving as the secondary candidates include:
a first colony detection parameter corresponding to the one colony detection result selected by the selection section from among the plurality of colony detection parameters serving as the primary candidates;
a second colony detection parameter larger than the first colony detection parameter; and
a third colony detection parameter smaller than the first colony detection parameter.

10. The colony counting device according to claim 6, further comprising
a calculation section that calculates the plurality of colony detection results using the different colony detection parameters,
wherein the calculation section calculates a plurality of colony detection results in advance using a plurality of colony detection parameters serving as primary candidates and a plurality of colony detection parameters serving as secondary candidates, and stores the plurality of colony detection results in a storage section,
the display processing section reads a plurality of colony detection results obtained using the plurality of colony detection parameters serving as the primary candidates from the storage section and displays the plurality of read colony detection results on the display section,
the selection section selects one colony detection result according to an operation of the user from among the plurality of colony detection results obtained by applying the plurality of colony detection parameters serving as the primary candidates and displayed on the display section,
the display processing section reads the one colony detection result and a plurality of colony detection results obtained using the plurality of colony detection parameters serving as the secondary candidates from the storage section and displays the read one colony detection result and the plurality of read colony detection results on the display section,
the selection section selects one colony detection result according to an operation of the user from among the one colony detection result and the plurality of colony detection results, obtained by applying the plurality of colony detection parameters serving as the secondary candidate, displayed on the display section, and
the counting section applies the colony detection parameter used to obtain the one colony detection result selected by the selection section to the image of the test individual and counts a number of colonies included in the image of the test individual.

11. The colony counting device according to claim 1, wherein
when a first type colony detection parameter is decided and stored in a saving section in response to an operation of the user,
the display processing section displays a plurality of colony detection results obtained by applying different second type colony detection parameters to the image of the test individual in a comparable manner while applying the first type colony detection parameter in common to the image of the test individual,
the selection section selects one colony detection result according to an operation of the user from among the plurality of colony detection results obtained by applying the different second type colony detection parameters and displayed on the display section, and
the counting section applies the first type colony detection parameter and the second type colony detection parameter used to obtain the one colony detection result selected by the selection section to the image of the test individual and counts a number of colonies included in the image of the test individual.

12. The colony counting device of claim 11, wherein the first type colony detection parameter is any of
a binarization sensitivity serving as a reference when a binarized image used to detect the colonies from the image of the test individual is generated,
on/off of shading correction applied to the image of the test individual, and
an intensity of noise reduction processing applied to the image of the test individual.

13. The colony counting device of claim 11, wherein the second type colony detection parameter is any of
on/off of reduction processing of reducing a small particle from the image of the test individual,
on/off of reduction processing of reducing an abnormal object from the image of the test individual, and
on/off of reduction processing of reducing a large particle from the image of the test individual.

14. The colony counting device according to claim 1, wherein
the display processing section is configured to display a first screen and a second screen on the display section,
the first screen is a screen that displays the plurality of colony detection results, and
the second screen is a screen for adjusting another colony detection parameter different from the colony detection parameter corresponding to the one colony detection result selected by the operation of the user on the first screen.

15. The colony counting device according to claim 14, wherein the colony detection parameter adjusted through the first screen is adjusted by selecting the one colony detection result from among the plurality of colony detection results obtained by applying discretely adjusted image processing parameters.

16. The colony counting device according to claim 14, wherein the second screen is a screen for receiving adjustment of colony detection parameters having consecutive values.

17. The colony counting device according to claim 1, wherein
the display processing section is configured to cause the display section to display a result display screen including:
the one colony detection result selected by the selection section according to the operation of the user;
identification information of the test individual corresponding to the one colony detection result;
a culture condition for the test individual; and
the number of colonies.

18. The colony counting device according to claim 17, wherein
the display processing section displays an adjustment screen, which indicates the plurality of colony detection results in a comparable manner, on the display section,
the display processing section transitions from the adjustment screen to the result display screen when the colony detection parameter used to obtain the one colony detection result selected by the selection section through the adjustment screen is stored in a saving section,
the display processing section transitions from the result display screen to the adjustment screen when a transition to the adjustment screen is instructed on the result display screen, and
the selection section receives re-adjustment of the colony detection parameter through the adjustment screen.

19. A control method for a colony counting device, the control method comprising:
acquiring an image of a test individual obtained by capturing the test individual;
displaying a plurality of colony detection results obtained by applying different colony detection parameters to the acquired image of the test individual in a comparable manner on a display section;
selecting one colony detection result from among the plurality of colony detection results displayed on the display section according to an operation of a user; and
storing the colony detection parameter used to obtain the one selected colony detection result as an official colony detection parameter.

20. A control method for a colony counting device, the control method comprising:
a first step of displaying a plurality of colony detection results, obtained by applying different first type colony detection parameters to an image of a test individual acquired by an acquisition section, on a display section in a comparable manner, selecting one colony detection result according to an operation of the user from among the plurality of colony detection results displayed on the display section, and storing a first type colony detection parameter used to obtain the selected one colony detection result in a storage device; and
a second step of displaying a plurality of colony detection results, obtained by applying the first type colony detection parameter applied to the one colony detection result and different second type colony detection parameters to the image of the test individual acquired by the acquisition section, on the display section in a comparable manner, selecting one colony detection result according to an operation of the user from among the plurality of colony detection results displayed on the display section, and storing the second type colony detection parameter used to obtain the selected one colony detection result in the storage device.
